(19) United States
(12) Patent Application Publication
(10) Pub. No.: US 2013/0210807 A1
(43) Pub. Date: Aug. 15, 2013

(54) Tricyclic Compounds as Allosteric Modulators of Metabotropic Glutamate Receptors.

(71) Applicants: Nigel J Liverton, Harleysville (GB); Christelle Boléa, Geneva (CH); Sylvain Celanire, Geneva (CH); Yunfu Luo, Shanghai (CN)

(72) Inventors: Nigel J Liverton, Harleysville (GB); Christelle Boléa, Geneva (CH); Sylvain Celanire, Geneva (CH); Yunfu Luo, Shanghai (CN)

(21) Appl. No.: 13/809,579

(22) PCT Filed: Jul. 9, 2011
(86) PCT No.: PCT/US11/01201

§ 371 Date: Mar. 25, 2013

Related U.S. Application Data
(60) Provisional application No. 61/364,195, filed on Jul. 14, 2010.

(51) Int. Cl. C07D 513/04; A61K 45/06; A61K 31/55; A61K 31/433; C07D 513/14; A61K 31/506

(52) U.S. Cl. CPC C07D 513/04; C07D 513/14; A61K 31/506; A61K 31/55; A61K 31/433; A61K 45/06
USPC 514/215; 544/331; 544/230; 544/320; 540/578; 548/128; 514/275; 514/272; 514/361; 514/217; 514/224.8

(57) ABSTRACT

The present invention describes and claims compounds of the Structural Formula I, Structural Formula II, or Structural Formula III: wherein R¹, R², R³ and R^{3′} are —H or methyl, or R³ and R³ taken together form a double bond, or R^{3′} is —H and R² and R³ taken together form a spiro-cyclopropyl substituent, R⁴ is —H or —F, and R5 is —H, methyl, —CI or —Br, Formula II wherein R¹ is —H, ethyl-, isopropyl-, cyclopropyl-, methyl- or methoxy-, R⁴ is —H or —F, and “Y” is: (a) —CH₂—; (b) —CR⁶H-0-CR⁷R⁸—, wherein R⁶, R⁷, and R⁸ are independently —H or methyl; (c) —CR⁶H—N(R⁹)—CR⁷R⁸—, wherein R⁶, R⁷, and R⁸ are independently —H or methyl; (d) —CH₂—C(R⁹)(R¹⁰)—C(R⁷)(R⁸)—, wherein R⁷, R⁸, R⁹ and R¹⁰ are independently —H or -methyl, or both R⁷ and R⁸ are —F, R⁹ and R¹⁰ are independently —H or -methyl, or both R⁹ and R¹⁰ are —F, or R⁹ and R¹⁰ taken together are (0=), which together with the carbon to which they are attached forms a carbonyl group.

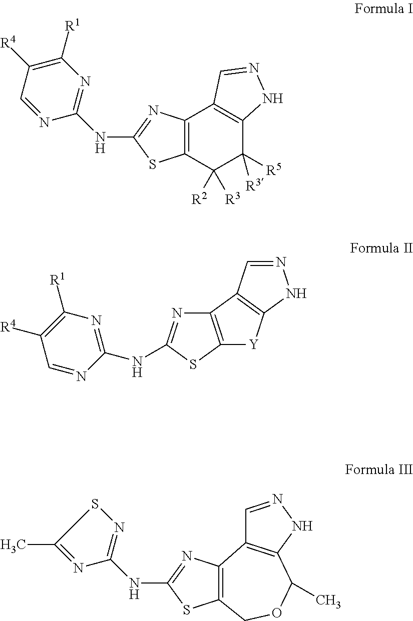

### FIELD OF THE INVENTION

This application discloses and claims novel compounds which have activity as positive allosteric modulators (PAMs) of metabotropic glutamate receptors, subtype 4 (mGluR4 receptors) and their use in preventing, managing, or treating diseases of the central nervous system and disorders modulated by mGluR4 receptors.

### BACKGROUND OF THE INVENTION

The excitatory amino acid L-glutamate (referred to herein also simply as glutamate) through its many receptors mediates most of the excitatory neurotransmissions within the mammalian central nervous system (CNS). Accordingly, glutamate is the major amino-acid transmitter in the mammalian CNS. The excitatory amino acids, including glutamate, are of great physiological importance, playing a role in a variety of physiological processes, for example, long-term potentiation (learning and memory), the development of synaptic plasticity, motor control, respiration, cardiovascular regulation, and sensory perception.

Glutamate is at the center of several different neurological and psychiatric diseases, where there is an imbalance in glutamatergic neurotransmission. Glutamate acts via at least two distinct classes of receptors. One class is composed of the ionotropic glutamate (iGlu) receptors that act as ligand-gated ion channels, namely the NMDA, AMPA and kainate receptors which are responsible for fast excitatory transmission (Nakanishi et al., (1998) Brain Res. Rev., 26:230-235). Glutamate mediates synaptic neurotransmission through the activation of ionotropic glutamate receptor channels (iGluRs). Glutamate is thought to regulate fast neuronal transmission within the synapse of two connecting neurons in the CNS via activation of the iGlu receptors.

The second general type of receptor is the G-protein, or secondary messenger-linked “metabotropic” glutamate (mGluR) receptors, which have a more modulatory role contributing to fine-tuning of synaptic efficacy. The mGluRs are G protein-coupled receptors (GPCRs) with seven-transmembrane spanning domains and belong to GPCR family along with the calcium-sensing GABAb and pheromone receptors. The mGluR family is composed of eight members. They are classified into three groups (group I comprising mGluR1 and mGluR5; group II comprising mGluR2 and mGluR3; and group III comprising mGluR4, mGluR6, mGluR7 and mGluR8) according to sequence homology, pharmacological profile and nature of intracellular signalling cascades activated (Schoepp et al., (1999) Neuropharmacology, 38:1431-1476). Glutamate activates the mGluRs through binding to the large extracellular amino-terminal domain of the receptor, herein called the orthosteric binding site. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McConald and Johnson, Brain Research Reviews, 15, 41 (1990). This activation induces a conformational change of the receptor which results in the activation of the G-protein and intracellular signalling pathways.

In the central nervous system, mGluR4 receptors are expressed most intensely in the cerebellar cortex, basal ganglia, sensory relay nuclei of the thalamus and hippocampus (Bradley et al., (1999) Journal of Comparative Neurology, 407:33-46; Corti et al., (2002) Neuroscience, 110:403-420). The mGluR4 subtype is negatively coupled to adenylate cyclase via activation of the Gai/o protein. It is expressed primarily on presynaptic terminals, functioning as an autoreceptor or heteroceptor. Activation of mGluR4 leads to decreases in transmitter release from presynaptic terminals (Corti et al., (2002) Neuroscience, 110:403-420; Millan et al., (2002) Journal of Biological Chemistry, 277:47796-47803; Valenti et al., (2003) Journal of Neuroscience, 23:7218-7226). Orthosteric agonists of mGluR4 are not selective and activate the other Group III mGluRs (Schoepp et al., (1999) Neuropharmacology, 5, 38:1431-1476). The Group III orthosteric agonist L-AP4 was able to reduce motor deficits in animal models of Parkinson's disease (Valenti et al., (2003) J. Neurosci., 23:7218-7226) and decrease excitotoxicity (Bruno et al., (2000) J. Neurosci., 20; 6413-6420) and these effects appear to be mediated through mGluR4 (Marino et al., (2005) Curr. Topics Med. Chem., 5:885-895). In addition to L-AP4, ACPT-1, another selective group III mGluR agonist has been shown to cause a dose-and-structure dependent decrease in haloperidol-induced catalepsy and attenuated haloperidol-increased Proenkephalin mRNA expression in the striatum (Konieczny et al., (2007) Neuroscience, 145:611-620). Furthermore, Lopez et al. (2007, J. Neuroscience, 27:6701-6711) have shown that bilateral infusions of ACPT-I or L-AP4 into the globus pallidus fully reversed the severe akinetic deficits produced by 6-hydroxydopamine lesions of nigrostriatal dopamine neurons in a reaction-time task without affecting the performance of controls. In addition, the reversal of haloperidol-induced catalepsy by intrapallidal ACPT-1 was prevented by concomitant administration of a selective group III receptor antagonist (RS)-alpha-cyclopropyl-4-phosphonophenylglycine.

The opposite effects produced by group III mGluR activation in the SNr strongly suggest a role of mGluR4 rather than others mGluR receptor sub-types in normalizing basal ganglia activity (Lopez et al. 2007). These results suggest that, among mGluR subtypes, mGluR4 is believed to be the most interesting novel drug target for the treatment of Parkinson's disease (for a review see Conn et al., (2005) Nature Review Neuroscience, 6:787-798).

Symptoms of Parkinson's disease appear to be due to an imbalance in the direct and indirect output pathways of the basal ganglia and reduction of transmission at the inhibitory GABAergic striato-pallidal synapse in the indirect pathway may result in alleviation of these symptoms (Marino et al., (2002) Amino Acids, 23:185-191). mGluR4 is more abundant in striato-pallidal synapses than in striato-nigral synapses, and its localization suggests it possibly functions as a presynaptic heteroreceptor on GABAergic neurons (Bradley et al., (1999) Journal of Comparative Neurology, 407:33-46). Further, this suggests that selective activation or positive modulation of mGluR4 would decrease GABA release in this synapse thereby decreasing output of the indirect pathway and reducing or eliminating Parkinson's disease symptoms. Classical treatment of Parkinsonism typically involves the use of _{Levo}dopa combined with carbidopa (SINEMET™) or benserazide (MADOPAR™). Dopamine agonists such as bromocriptine (PARLODEL™), lisuride and pergolide (CELANCE™) act directly on dopamine receptors and are also used for the treatment of Parkinsonism. These molecules have the same side-effect profile as _{Levo}dopa. A new avenue for developing selective compounds acting at mGluRs is to identify molecules that act through allosteric mechanisms, modulating the receptor by binding to a site different from the highly conserved orthosteric binding site. Positive allosteric modulators of mGluRs have emerged recently as novel pharmacological entities offering this attractive alternative. This type of molecule has been discovered for mGluR1, mGluR2, mGluR4, mGluR5, mGluR7 and mGluR8 (Knoflach F. et al. (2001) Proc. Natl. Acad. Sci. USA, 98:13402-13407; Johnson M. P. et al., (2002) Neuropharmacology, 43:799-808; O'Brien J. A. et al., (2003) Mol. Pharmacol., 64:731-740; Johnson M. P. et al., (2003) J. Med. Chem., 46:3189-3192; Marino M. J. et al., (2003) Proc. Natl. Acad. Sci. USA, 100:13668-13673; Mitsukawa K. et al., (2005) Proc. Natl. Acad. Sci. USA, 102(51):18712-18717; Wilson J. et al., (2005) Neuropharmacology, 49:278; for a review see Mutel V., (2002) Expert Opin. Ther. Patents, 12:1-8; Kew J. N., (2004) Pharmacol. Ther., 104(3):233-244; Johnson M. P. et al., (2004) Biochem. Soc. Trans., 32:881-887; recently Ritzen A., Mathiesen, J. M. and Thomsen C., (2005) Basic Clin. Pharmacol. Toxicol., 97:202-213).

Particular molecules have been described as mGluR4 positive allosteric modulators (Maj et al., (2003) Neuropharmacology, 45:895-906; Mathiesen et al., (2003) British Journal of Pharmacology, 138:1026-1030). It has been demonstrated that such molecules have been characterized in in vitro systems as well as in rat brain slices where they potentiated the effect of L-AP4 in inhibiting transmission at the striatopallidal synapse. These compounds do not activate the receptor by themselves (Marino et al., (2003) Proc. Nat. Acad. Sci. USA, 100:13668-13673). Rather, they enable the receptor to produce a maximal response to a concentration of glutamate or the Group III orthosteric agonist L-AP4 which by itself induces a minimal response.

PHCCC, a positive allosteric modulator of mGluR4 which is not active on other mGluRs (Maj et al., (2003) Neuropharmacology, 45:895-906), has been shown to be efficacious in animal models of Parkinson's disease thus representing a potential novel therapeutic approach for Parkinson's disease as well as for other motor disorders and disturbances (Marino et al., (2003) Proc. Nat. Acad. Sci. USA, 100:13668-13673), neurodegeneration in Parkinson's disease (Marino et al., (2005) Curr. Topics Med. Chem., 5:885-895; Valenti et al., (2005) J. Pharmacol. Exp. Ther., 313:1296-1304; Vernon et al., (2005) Eur. J. Neurosci., 22:1799-1806, Battaglia et al., (2006) J. Neurosci., 26:7222-7229), and neurodegeneration in Alzheimer's disease or due to ischemic or traumatic insult (Maj et al., (2003) Neuropharmacology, 45:895-906). PHCCC has been shown also to be active in an animal model of anxiety (Stachowicz et al., (2004) Eur. J. Pharmacol., 498:153-156). Previously, ACPT-1 has been shown to produce a dose-dependent anti-conflict effect after intrahippocampal administration and anti-depressant-like effects in rats after intracerebroventricular administration (Tatarczynska et al., (2002) Pol. J. Pharmacol., 54(6):707-710). When injected intraperitoneally, ACPT-1 has been shown also to have anxiolytic-like effects in stress-induced hyperthermia, in mice in the elevated-plus maze and in rats in the Vogel conflict test (Stachowicz et al., (2009) Neuropharmacology, 57(3):227-234).

Activation of mGluR4 receptors which are expressed in a- and F-cells in the islets of Langerhans inhibits glucagon secretion. Molecules activating or potentiating the agonist activity of these receptors may offer effective treatment for hyperglycemia, one of the symptoms of type 2 diabetes (Uehara et al., (2004) Diabetes, 53:998-1006).

The b-chemokine RANTES is importantly involved in neuronal inflammation and has been implicated in the pathophysiology of multiple sclerosis. Activation of Group III mGluRs with L-AP4 reduced the synthesis and release of RANTES in wild-type cultured astrocytes, whereas the ability of L-AP4 to inhibit RANTES was greatly decreased in astrocyte cultures from mGluR4 knockout mice (Besong et al., (2002) Journal of Neuroscience, 22:5403-5411). These data suggest that positive allosteric modulators of mGluR4 may be an effective treatment for neuroinflammatory disorders of the central nervous system, including multiple sclerosis and related disorders.

Two different variants of the mGluR4 receptor are expressed in taste tissues and may function as receptors for the umami taste sensation (Monastyrskaia et al., (1999) Br. J Pharmacol., 128:1027-1034; Toyono et al., (2002) Arch. Histol. Cytol., 65:91-96). Thus positive allosteric modulators of mGluR4 may be useful as taste agents, flavour agents, flavour enhancing agents or food additives. There is anatomical evidence that the majority of vagal afferents innervating gastric muscle express group III mGluRs (mGluR4, mGluR6, mGluR7 and mGluR8) and actively transport receptors to their peripheral endings (Page et al., (2005) Gastroenterology, 128:402-10). Recently, it was shown that the activation of peripheral group III mGluRs inhibited vagal afferents mechanosensitivity in vitro which translates into reduced triggering of transient lower oesophagal sphincter relaxations and gastroesophageal reflux in vivo (Young et al., (2008) Neuropharmacol, 54:965-975). Labelling for mGluR4 and mGluR8 was abundant in gastric vagal afferents in the nodose ganglion, at their termination sites in the nucleus tractus solitarius and in gastric vagal motoneurons. These data suggest that positive allosteric modulators of mGluR4 may be an effective treatment for gastro-esophageal reflux disease (GERD) and lower esophageal disorders and gastro-intestinal disorders.

International patent publication WO2005/007096 describes mGluR4 receptor positive allosteric modulator useful, alone or in combination with a neuroleptic agent, for treating or preventing movement disorders. However, none of the specifically disclosed compounds are structurally related to the compounds of the invention.

More recently, new mGluR4 receptor positive allosteric modulators (PAMs) have been described: pyrazolo[3,4-d]pyrimidine derivatives (Niswender et al., (2008) Bioorganic & Medicinal Chemistry Letters, 18(20):5626-5630), functionalized benzylidene hydrazinyl-3-methylquinazoline and bis-2,3-dihydroquinazolin-4(1H)-one (Williams et al., (2009) Bioorganic 10 c & Medicinal Chemistry Letters, 19:962-966) and heterobiarylamides (Engers et al, (2009) Journal of Medicinal Chemistry, 52 (14), pp 4115-4118). Niswender et al., described (±)-cis-2-(3,5-dichlorophenylcarbamoyl)cyclohexane carboxylic acid ((2008) Molecular Pharmacology, 74(5):1345-1358), as a positive allosteric modulator of mGluR4 also having agonist activity. This moderately active molecule has demonstrated evidence of efficacy following icy injection in rat models of Parkinson's disease. International patent publications WO2009/010454 and WO2009/010455 have mentioned amido derivatives and novel heteroaromatic derivatives, respectively, as positive allosteric modulators of metabotropic glutamate receptors. East et al. have also examined heteroaromatic derivatives as PAMs of mGlu receptors (East, Stephen P. et al., (2010) Expert Opin. Ther. Patents, 20(3): 441-445). The Re-exploration of the PHCCC scaffold was described by Williams, R. et al. in (2010) ACS Chemical Neuroscience, 1(6): 411-419.

There is strong motivation that mGluR4 receptor positive allosteric modulators are useful for treating or preventing a condition in a mammal, including a human, which treatment is affected or facilitated by the neuromodulatory effect of mGluR4 modulators. In the case of the treatment of movement disorders such as Parkinson's disease, the compounds of the invention can be used alone or in combination with an agent selected from the group consisting of: levodopa, levodopa with a selective extracerebral decarboxylase inhibitor, carbidopa, entacapone, a COMT inhibitor, a dopamine agonist, an anticholinergic, a cholinergic agonist, a butyrophenone neuroleptic agent, a diphenylbutylpiperidine neuroleptic agent, a heterocyclic dibenzazepine neuroleptic agent, an indolone neuroleptic agent, a phenothiazine neuroleptic agent, a thioxanthene neuroleptic agent, an NMDA receptor antagonist, an MAO-B inhibitor, an mGluR5 antagonist or an A_{2A} antagonist.

International patent publication WO2006/040279 describes compounds which are PI3 kinase modulator but does not describe the compounds of the present invention.

### SUMMARY OF THE INVENTION

Given the importance of agents which act as mGluR4 PAM compounds, what is needed are compounds which can act as mGluR4 PAMs with high activity. This need is met by the present invention which in one aspect are compounds of the structure of Formula I:

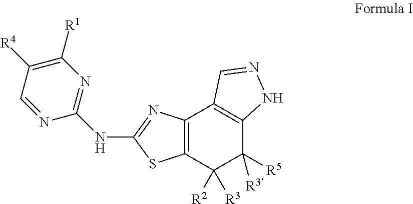

wherein R¹, R², R³, R^{3′} and R⁴ are defined in accordance with the substituents set forth in Table I. As noted in the table, in some cases, R³ and R^{3′} are taken together to form a double bond, thus the Structure of Formula I is the structure of Formula I′

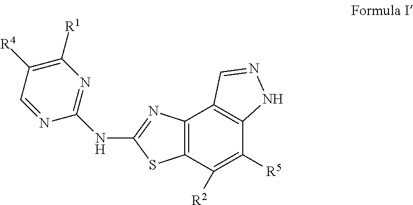

As noted also in Table I, in some instances, R² and R³ are taken together to form a spiro-cyclopropyl ring substituent, thus the Structure of Formula I is the structure of Formula 1″

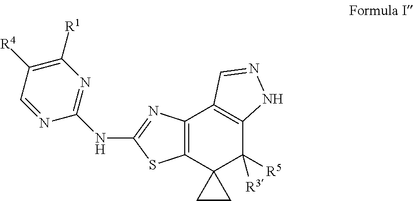

TABLE I

| Cmpd | | | | | | EC₅₀ |
|---|---|---|---|---|---|---|
| No. | R¹ | R² | R³/R^{3′} | R⁴ | R⁵ | (nM)*. |
| 1 | —CH₃ | —CH₃ | —CH₃/—H | —H | —H | 326 |
| 2 | —CH₃ | —H | Double* | —H | —H | 28 |
| 3 | —H | —H | Double* | —H | —H | 9 |
| 4 | —H | —CH₃ | —CH₃/—H | —F | —H | 753 |
| 5 | —CH₃ | —CH₃ | —CH₃/—H | —F | —H | 645 |
| 6 | —H | —H | Double* | —H | —Br | 1 |
| 7 | —H | —H | Double* | —H | —Cl | 2 |
| 8 | —CH₃ | —H | Double* | —H | —CH₃ | 129 |
| 9 | —H | —H | —H/—H | —H | —CH₃ | 2 |
| 10 | —CH₃ | —H | —H/—H | —H | —CH₃ | 36 |

| | | | | | |
|---|---|---|---|---|---|
| 11 | —CH₃ | R^{2 and} R³ taken together form a | —H | —H | 545 |
| | | cyclopropyl ring, R^{3′} is —H | | | |
| 12 | —H | R^{2 and} R³ taken together form a | —H | —H | 35 |
| | | cyclopropyl ring, R^{3′} is —H | | | |
| 13 | —H | R^{2 and} R³ taken together form a | —F | —H | 93 |
| | | cyclopropyl ring, R^{3′} is —H | | | |

*R³ and R^{3′} are taken together to form a double bond

Another aspect of the invention are the compounds of Formula II

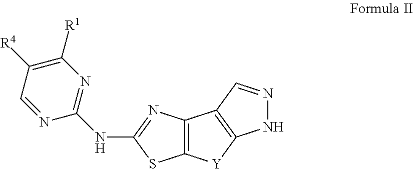

wherein R¹, R⁴ and “Y” are defined in accordance with the substituents set forth in Table II.

TABLE II

| Cmpd. No. | Y | R¹ | R⁴ | EC₅₀ (nM). |
|---|---|---|---|---|
| 14 | —CH₂— | —H | —H | 1425 |
| 15 | —CH₂OCH₂— | —H | —F | 8 |
| 16 | —CH₂OCH₂— | —H | —H | 7 |
| 17 | —CH₂OCH₂— | —CH₃ | —H | 10 |
| 18 | —CH₂-O-C(CH₃)H— | —CH₃ | —H | 3 |
| | —CH₂—O—C*(CH₃)H— | | | ND¹ |
| | * “R” isomer | | | |
| | —CH₂—O—C**(CH₃)H— | | | ND¹ |
| | ** “S”—isomer | | | |
| 19 | —CH₂OCH₂— | —OCH₃ | H | 28 |
| 20 | —CH₂—O—C(CH₃)H— | —CH₂CH₃ | H | 173 |
| 21 | —CH₂—O—C(CH₃)H— | —CH₃— | —F | 27 |
| 22 | —CH₂—O—C(CH₃)H— | —H— | —F | ND² |
| 23 | —HC(CH₃)—O—CH₂— | —CH₃ | —H | 186 |
| 24 | —CH(CH₃)—O—C(CH₃)H— | —CH₃ | —H | 121 |
| 27 | —CH₂—NH—CH₂— | —H | —H | 994 |
| 28 | —CH₂—N(CH₃)—CH₂— | —CH₃ | —H | 2375 |
| 29 | —CH₂—CH₂—CH₂— | —H | —F | 39 |
| 34 | —CH₂—CH₂—CF₂— | —CH₃ | —H | 56 |
| 36 | —CH₂—CH₂—C(O)— | —CH₃ | —H | 1500 |
| 40 | —CH₂—CFH—CH₂— | —CH₃ | —H | 20 |

¹EC₅₀ is expected to be at least similar to that of the racemate.
²EC₅₀ is expected to be less than or equal to 50 micromolar.

In another aspect of the invention is the compound of Formula 38,

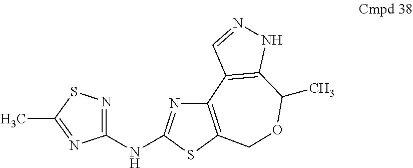

EC₅₀ for Cmpd 38 was determined to be 70 nanomolar.

In another aspect the invention provides for the prevention, treatment, or management of a disease or disorder modulated by mGluR4 receptors by administering a pharmaceutical formulation comprising one or more compounds described above in Table I, Table II, or the compound of Formula 38 in an amount providing a therapeutic level of said one or more compounds.

### DETAILED DESCRIPTION

Compounds of Table I may generally be prepared using one or more, of the following synthetic schemes. Examples of the preparation of the compounds of Table I are related further below.

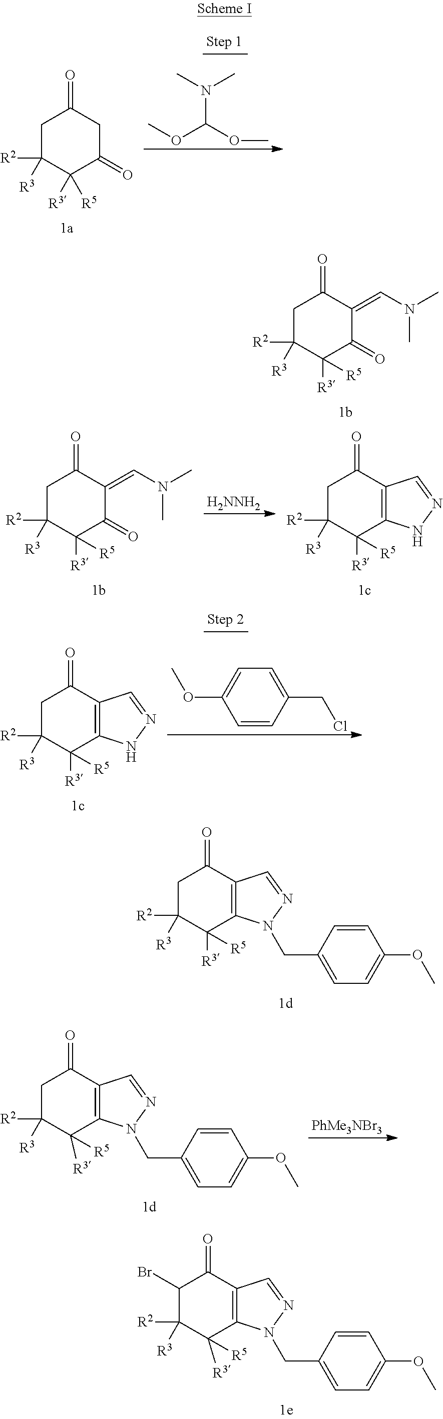

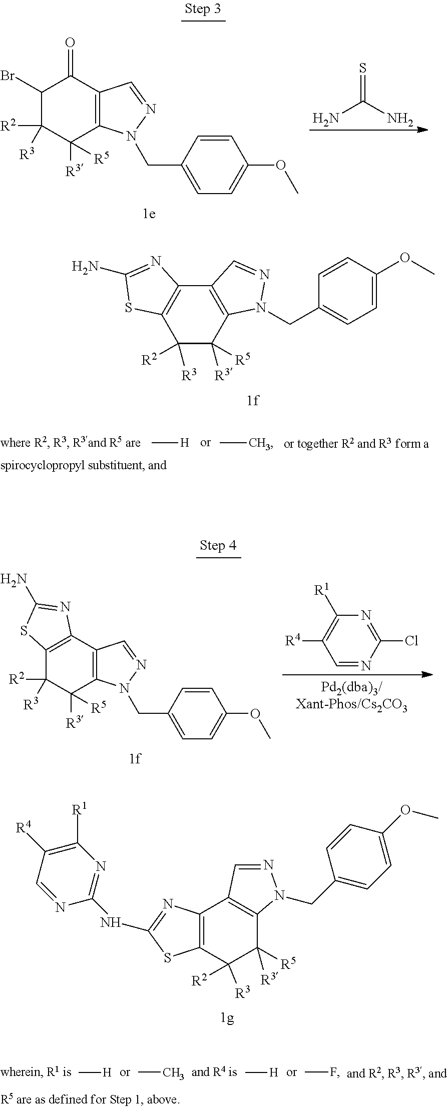

With reference to Scheme I, Steps 1 to 4, when R² and R³ together form a cyclopropyl substituent, the starting material of (1a) has the structure of Formula 4.

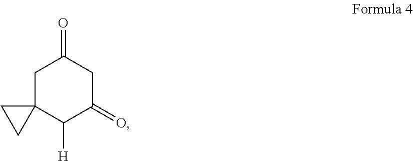

and these substituents are maintained throughout the reaction.

With reference to Scheme I, in Step 1, a diketone of Formula 1a is reacted with dimethylmethanamine to form an intermediate of Formula 1b. The intermediate of Formula 1b thus formed is further reacted with hydrazine to yield a corresponding indazole-one intermediate of Formula 1c.

As shown in Step 2 of Scheme I, after preparation of the indazole-one intermediate of Formula 1c the reactive nitrogen in the pyrazole ring portion of the indazole-one is protected, then subsequently the protected indazole-one is halogenated on the cyclohexenone portion thereby providing an intermediate of Formula 1e.

As shown in Step 3 of Scheme I, an intermediate of Formula 1e is then reacted with thiourea to provide a corresponding thiazole substituted indazole of Formula 1f:

In Step 4 of Scheme I, a metal-assisted amination of an appropriately substituted chloropyrimidine is carried out using an intermediate of Formula 1f in the presence of a palladium catalyst to provide a compound of Table 1. Alternatively, where the cyclohexenone ring of intermediate 1e is unsubstituted and an unsubstituted pyrimidine substituent is desired, Steps 3 and 4 can be carried out using 1-pyrimidin-2-ylthiourea, as illustrated in Example 14, Step F, to provide a pyrimidine-substituted tricyclic intermediate analogs to compound 1g, wherein R¹ to R⁵ are protons.

In some embodiments, it is desired to provide a compound of the invention wherein the R⁵ substituent is a halogen, for example, —Br or —Cl, for example, compounds 6 and 7 in Table 1. In general these compounds can be provided by carrying out the reactions of Scheme I, Steps 1 and 2, then in step 3 adding Pd/C to the reaction mixture along with thiourea (wherein substituents R³ and R^{3′} of the compound of Formula 1e are “—H”) to afford the aromatized intermediate (1f*):

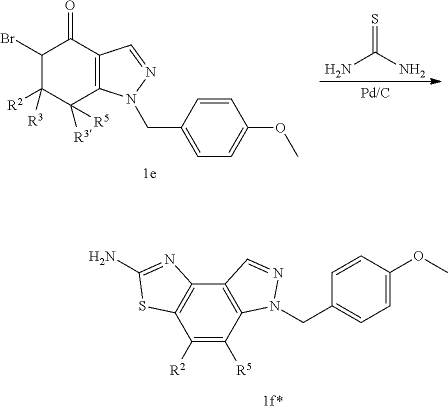

The intermediate 1f* is then reacted with an appropriately substituted chloropyrimidine reagent in the analogous manner shown in Scheme I, Step 4, to yield the compound of Formula 1g*, which is subsequently halogenated using N-chloro- or N-bromosuccinimide as shown below in Scheme II.

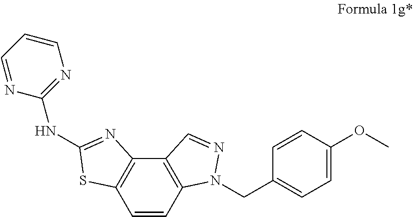 [SMILES: COc1ccc(Cn2ncc3c4nc(Nc5ncccn5)sc4ccc32)cc1]

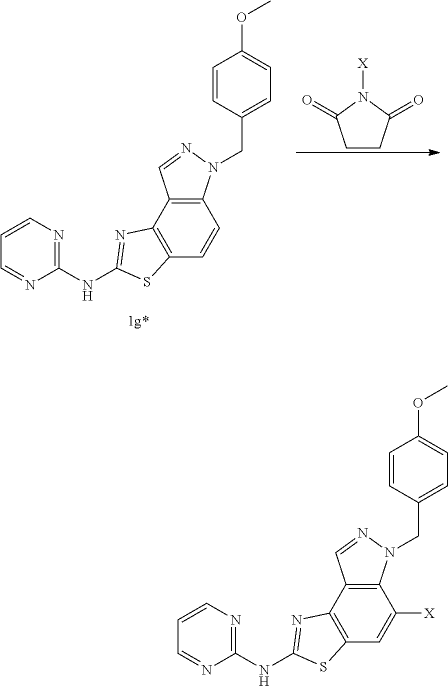

An intermediate analogous to compounds of Formula 1b (Scheme I, above) for use in providing the compound of Table II (above), wherein “Y” defines a 5-carbon member ring, may generally be prepared using the methodology shown in Scheme III, wherein, in Step 1 of Scheme III is carried out in the same manner as Step 1 in Scheme I, above, with the cyclopentanedione of Formula 2a substituted for the cyclohexanedione of Formula 1a shown in Scheme I.

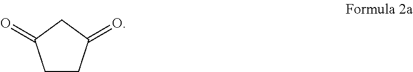

Where “Y” in compounds of Table II defines a 7-member ring, in general, a suitably substituted bromo-pyrazole-substituted cycloheptanone intermediate, for example, intermediate 29j, below, is prepared for reaction with thiourea to provide an analog of intermediate 1f, and subsequently reacted with an appropriately substituted chloropyrimidine in a subsequent step analogous to Step 4 of Scheme I (above) to yield the desired product.

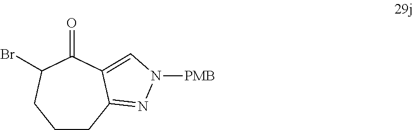

Intermediates useful in providing compounds 15 to 29 of Table II, are provided in reaction steps A or B shown in Scheme III, depending upon whether the 7-member ring contains oxygen or nitrogen as the heteroatom.

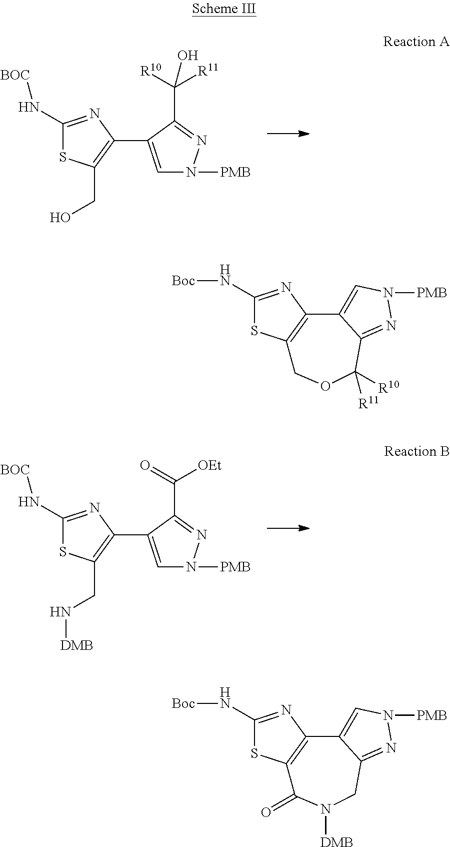

The intermediates provided in the reactions shown in Scheme III are further derivatized using reaction analogous to those described above in Scheme I to provide various of the compounds listed in Table II.

The Examples below illustrate various methods of providing the intermediates used in these reaction steps, in addition to illustrating how the products of Reactions A and B are further reacted to provide the desired compound.

Further preparative methods and useful procedures for the provision of compounds of the invention are provided in the Examples below. Other aspects of the invention will also be appreciated from reading the Examples.

### Analytical Equipments and Methods:

LC-MS were recorded on Agilent 1200 RRLC equipped with 6110 MSD with the following conditions: Reversed phase HPLC was carried out on Zorbax SB-C18 analytical column (5 μm, 2.1×50 mm) from Agilent, with a flow rate of 0.8 mL/min. The gradient conditions used are: 90% A (water+0.1% of trifluoroacetic acid), 10% B (acetonitrile+0.05% of trifluoroacetic acid) to 100% B at 3.5 minutes, maintained until 4.0 minutes and then equilibrated to initial conditions beginning at 4.01 minutes until 4.5 minutes. Injection volume 2-5 μL. ES MS detector was used, acquiring in positive ionization mode.

All mass spectra were taken under electrospray ionisation (ESI) methods.

Preparative HPLC was conducted using a Gilson GX-281 preparative HPLC (322 Binary Gradient Module, 156 UV/Visible detector GX-281 injector/fraction collector) Phenomenex Synergi Max-Rp (C12, 30×150 mm, 4 μm) or Kromasil Eternity (C18, 30×150 mm, 5 μm) columns and H₂O+0.1% TFA and CH₃CN as eluents. Gradients used cover the range from 0% CH₃CN to 100% CH₃CN.

¹H NMR spectra were recorded on a Bruker Avance 400 MHz or Varian 400 MHz spectrometer. Chemical shifts are expressed in parts of million (ppm, δ units). Coupling constants are in units of hertz (Hz) Splitting patterns describe apparent multiplicities and are designated as s (singlet), d (doublet), t (triplet), q (quadruplet), quint (quintuplet), m (multiplet), br (broad).

The compounds provided by the present invention are believed to be positive allosteric modulators if mGluR4, and as such they do not appear to bind to the orthosteric glutamate recognition site. Accordingly, they do not activate the mGluR4 receptor by themselves, instead, the response of mGluR4 to a concentration of glutamate or mGluR4 agonist is increased when a compound of the invention is present. Accordingly, it is expected that the compounds of the invention will have an effect at mGluR4 by virtue of enhancing the function of the receptor.

mGluR4 Assay on HEK-Expressing Human mGluR4

Since, as explained above, the compounds of the present invention are positive allosteric modulators of mGluR4 receptors, which activity was assayed by detecting changes in intracellular Ca⁺² ion concentration using a Ca⁺²-sensitive fluorescent dye (Fluo4-(AM)) and a fluorometric imaging plate reader (FLIPR, Molecular Devices, Sunnyvale, Calif.) in accordance with the manufacturers instructions.

Accordingly, 24 hours prior to carrying out an analysis, human mGluR4 HEK-293 cells were plated out in black-walled, clear-bottomed, poly-L-ornithine-coated 384-well plates at a density of 25,000 cells/well in a glutamine/glutamate-free DMEM medium containing fetal bovine serum (10%), penicillin (100 units/mL) and streptomycin (100 micrograms/mL) at 37° C. under 5% CO₂. On the day of the assay, the medium was aspirated and the cells were loaded with a 3 micro-molar solution of Fluo4-AM (LuBioScience, Lucerne, Switzerland) in 0.03% pluronic acid. After maintaining the plate 1 hour at 37° C. under 5% CO₂ the non-incorporated dye was removed by washing the cell plate with the assay buffer and the cells were left in the dark at room temperature for six hours before evaluating. All assays were performed in a pH 7.4 buffered-solution containing 20 mM HEPES, 142 mM NaCl, 6 mM KCl, 1 mM MgSO₄, 1 mM CaCl₂, 0.125 mM sulfapyrazone, and 1% glucose.

Basal fluorescence was recorded over 10 seconds, then aliquots of various concentrations of a compound of the invention were added to the cells. Changes in fluorescence level were first monitored for 180 seconds in order to detect any agonist activity attributable to the compound being assayed. Then the cells were stimulated for an additional 110 seconds by that concentration of glutamate yielding 25% of the maximal response to glutamate (EC₂₅). Concentration-response curves of compounds of the invention were generated using Prism GraphPad software (Graph Pad Inc., San Diego, USA). Curves generated were fitted to a four-parameter logistic equation allowing the determination of EC₅₀ values:

Y=Bottom+(Top−Bottom)/(1+10^{logEC}^{₅₀₋}^{X)}*Hill Slope.

For each sample assayed, at least three independent measurements were performed. EC₅₀ values thus determined are reported for each of the compounds of the invention in the respective Tables 1 to 3 herein above.

Accordingly, the compounds of the invention are believed to be effective in the treatment, prevention, or management of neurological or psychiatric diseases or disorders associated with glutamate dysfunction which are amenable to treatment, prevention, or management by administration of a positive allosteric modulator. It is believed that the compounds of the invention can be incorporated into dosage forms which lend themselves to administration via the alimentary canal (oral), through mucosal tissue (for example, administration by absorption through tissues of the oral cavity, rectal, and vaginal mucosa), via dermal absorption, or via intramuscular or intravenous injection. International application no. PCT/EP2010/050304, filed Jan. 12, 2010 is incorporated by reference as if fully set forth herein for the purpose of illustrating various dosage forms suitable for the compounds of the present invention. It will be appreciated that other known methods of administration and other known dosage forms can be adopted for compounds of the present invention.

### Abbreviations Used in the Application

Unless otherwise defined herein, all abbreviations and chemical terms are meant to have their conventionally accepted meanings. Following is a list of specific abbreviations used in the application and their meaning:

AcOH Acetic acid

NH₄Cl Ammonium chloride

Ar Argon

CH₃CN Acetonitrile

NBS N-Bromosuccinimide

Boc t-butoxy carbonyl-(CH₃)₃C—O—C(O)—

Boc₂O di-t-butyl-dicarbonate ((CH₃)₃C—O—C(O))₂O

nBuOH n-Butanol

nBuLi n-Butyllithium

t-BuONO t-Butyl nitrite

Cs₂CO₃ Caesium carbonate

CuBr₂ Copper (II) bromide

DCM Dichloromethane

(i-Pr)₂NH Diisopropylamine

DMAP N,N-Dimethylaminopyridine

DMB 2,4-Dimethoxybenzyl

DMSO Dimethyl sulfoxide

DMF Dimethylformamide

EtOH Ethanol

EtOAc Ethyl acetate

Iodine

LiBH₄ Lithium borohydride

LDA Lithium diisopropylamide

MgSO4 Magnesium sulfate

MeOH Methanol

PMB 4-methoxybenzyl-

PMBCl 4-Methoxybenzyl chloride

TsNHNH₂ 4-Methylbenzenesulfonyl hydrazide

CH₃Li Methyllithium

N₂ Nitrogen

Pd/C 10% Palladium on charcoal

Pd₂(dba)₃ Tris(dibenzylideneacetone)dipalladium(0).

Pd(dppf)Cl₂ Dichloro-[1,1′-bis(diphenylphosphino)ferrocenyl]palladium (II)

Pd(PPh₃)₂Cl₂ Dichloro-bis(triphenylphosphine)palladium (II)

Pd(PPh₃)₄ Tetakis(triphenylphosphine) palladium (0)

PE Petroleum ether

K₂CO₃ Potassium carbonate

K₃PO₄ Potassium phosphate

r.t. Room temperature

NaBH₄ Sodium borohydride

Na₂CO₃ Sodium carbonate

NaHSO₃ Sodium hydrogen sulfite

NaOH Sodium hydroxide

Na₂SO₄ Sodium sulfate

H₂SO₄ Sulfuric acid

THF Tetrahydrofuran

TEA Triethylamine

TFA Trifluoroacetic acid

TfOH Trifluoromethanesulfonic acid

TLC Thin layer chromatography

H₂O Water

Xant-phos 4,5-Bis(diphenylphosphino)-9,9-dimethylxanthene

Structural representations of compounds and chemical nomenclature, unless otherwise specifically indicating particular stereochemistry, are meant to encompass all possible stereoisomers obtainable from the reaction shown.

It will be appreciated that various of the compounds provided by the reactions described herein, and the compounds listed in Tables I and II and Cmpd 38, can be provided as a salt, particularly a pharmaceutically acceptable salt, and in various crystalline and amorphous forms, including solvate crystalline and amorphous forms, in accordance with known methods. Accordingly, unless otherwise noted, identification of a particular compound is meant to encompass that compound in all crystalline and amorphous forms, including solvate forms, available from known methods. Examples of techniques for preparing crystalline and salt forms of compounds may be found in any addition of Remington, for example, The Science and Practice of Pharmacy, 21st edition.

### EXAMPLES

### Example 1

Example 1 describes preparation of Cmpd 1,4,4-dimethyl-N-(4-methylpyrimidin-2-yl)-5,6-dihydro-4H-[1,3]thiazolo[4,5-e]indazol-2-amine (Table I, compound no. 1).

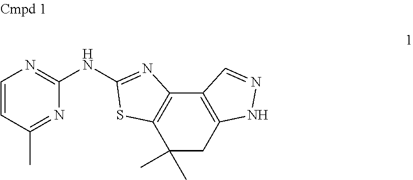 [SMILES: Cc1ccnc(Nc2nc3c(s2)C(C)(C)Cc2[nH]ncc2-3)n1]

### Example 1

### Step A

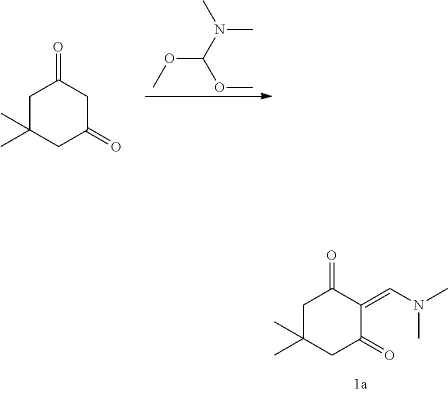

A solution of 5,5-dimethylcyclohexane-1,3-dione (60 g, 428 mmol) and 1,1-dimethoxy-N,N-dimethylmethanamine (68.2 mL, 514 mmol) was stirred at room temperature for 1 hour. The mixture was concentrated and triturated in cyclohexane to afford the title compound (38.5 g, 46%) as a pale yellow solid.

This compound was characterized by mass spectroscopy (MS) in accordance with the procedure described herein. Results of MS indicated MS (ESI): m/z 196 (M+H)⁺

### Example 1

### Step B

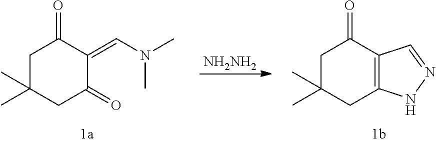

To a solution of 1a (38.5 g, 197 mmol) in EtOH (500 mL) at 0° C. was slowly added acetic acid (8.55 mL) followed by hydrazine monohydrate (11.3 mL, 197 mmol). The reaction mixture was then heated to reflux for 17 hrs. After cooling to r.t., the mixture was filtered and the filtrate concentrated to give the crude title product 1b (19.4 g, 60%).

This compound was characterized by mass spectroscopy (MS) in accordance with the procedure described herein. Results of MS indicated MS (ESI): m/z 165 (M+H)⁺

### Example 1

### Step C

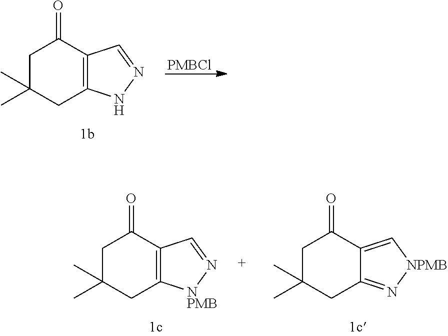

To a mixture of compound 1b (16.4 g, 100 mmol) and K₂CO₃ (27.6 g, 200 mmol) in CH₃CN (50 mL) was added PMBCl (18.7 g, 120 mmol) and the mixture heated to reflux for 6 hrs. After cooling to r.t., the mixture was diluted with H₂O (40 mL) and extracted with EtOAc (30 mL×3), the combined organic layers were dried over Na₂SO₄, filtered and the filtrate was concentrated in vacuo and purified by chromatography on silica to give the title compound 1c (13 g, 46%) and 1c′ (12 g, 42%).

This compound was characterized by proton NMR which yielded the following results:

¹H NMR (CDCl₃, 400 MHz): δ 7.64 (s, 1H), 7.14 (d, J=8.4 Hz, 2H), 6.82 (d, J=8.4 Hz, 2H), 5.12 (s, 2H), 3.73 (s, 3H), 2.62 (s, 2H), 2.25 (s, 2H), 1.01 (s, 6H).

¹H NMR (CDCl₃, 400 MHz): δ 7.82 (s, 1H), 7.01 (d, J=8.8 Hz, 2H), 6.78 (d, J=8.8 Hz, 2H), 5.13 (s, 2H), 3.70 (s, 3H), 2.50 (s, 2H), 2.24 (s, 2H), 0.99 (s, 6H).

### Example 1

### Step D

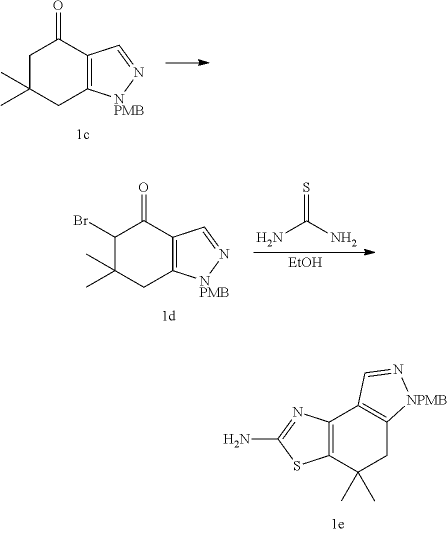

To a solution of ketone 1c (2.84 g, 10 mmol) in chloroform (15 mL) at r.t. was added trimethylphenylammomium tribromide (3.76 g, 10 mmol) and the reaction mixture heated to reflux for 2 hrs. After cooling to r.t., the mixture was diluted with H₂O (20 mL) and extracted with DCM (20 mL×2), the combined organic layers were dried over Na₂SO₄, filtered and the filtrate concentrated in vacuo to give crude product 1d. Thiourea (3.8 g, 50 mmol) and EtOH (15 mL) was added to the crude 1d and the mixture heated to reflux for 24 hrs. The mixture was cooled to r.t., concentrated in vacuo and 1N NaOH (20 mL) added to the residue, extracted with EtOAc (20 mL×3), the combined organic layers were washed with H₂O (10 mL) and brine (10 mL), dried over Na₂SO₄, concentrated in vacuo and purified by chromatography on silica to give product 1e (600 mg, 18%).

This compound was characterized by proton NMR and by mass spectroscopy (MS) in accordance with the procedures described herein. Results of NMR indicated ¹H NMR (CD₃OD, 400 MHz): δ 7.54 (s, 1H), 7.19 (d, J=8.8 Hz, 2H), 6.88 (d, J=8.8 Hz, 2H), 5.18 (s, 2H), 3.76 (s, 3H), 2.73 (s, 2H), 1.23 (s, 6H). Results of the MS yielded MS (ESI): m/z 341 (M+H)⁺

### Example 1

### Step E

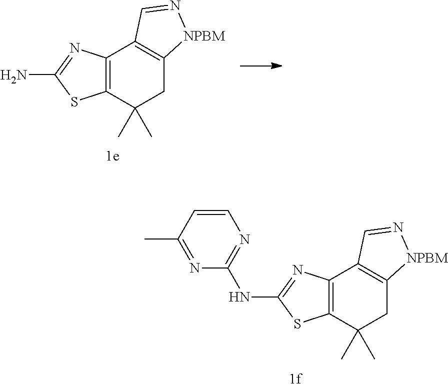

A mixture of compound 1e (68 mg, 0.2 mmol), 2-chloro-4-methylpyrimidine (31 mg, 0.24 mmol), Pd₂(dba)₃ (22.85 mg, 0.025 mmol), Xant-phos (28.9 mg, 0.05 mmol) and Cs₂CO₃ (98 mg, 0.3 mmol) in dioxane (3 mL) was heated to reflux under N₂ for 4 hrs. After cooling to r.t., the mixture was diluted with H₂O (10 mL) and extracted with EtOAc (10 mL×2), the combined organic layers were dried over Na₂SO₄, concentrated in vacuo and purified by preparative TLC (EtOAc: PE=2:1) to give product If (62.4 mg, 75%).

This compound was characterized by mass spectroscopy (MS) in accordance with the procedure described herein. Results of MS indicated MS (ESI): m/z 433 (M+H)⁺

### Example 1

### Step F

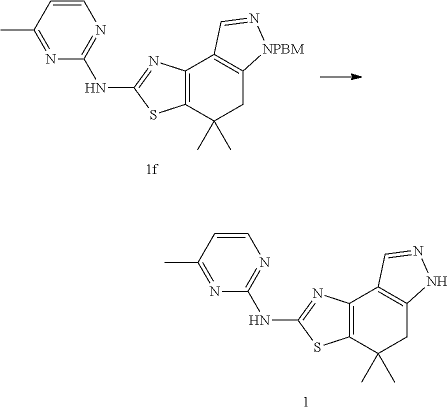

A solution of compound 1f (31.2 mg, 0.075 mmol) in TFA/TfOH (1 mL/0.1 mL) was stirred at 100° C. in a microwave for 0.5 h. After cooling to Et., the mixture was concentrated in vacuo and the residue purified by preparative HPLC to yield, with reference to Table 1, the compound of Example 1 (18.5 mg, 83%).

This compound was characterized by proton NMR (¹H NMR) and mass spectroscopy (MS) in accordance with the procedures described herein. Results of MS yielded ¹H NMR (CD₃OD, 400 MHz): δ 8.49 (d, J=5.2 Hz, 1H), 7.77 (s, 1H), 6.96 (d, J=5.6 Hz, 1H), 2.88 (s, 2H), 2.59 (s, 3H), 1.38 (s, 6H).

This compound was characterized by mass spectroscopy (MS) in accordance with the procedure described herein. Results of MS indicated MS (ESI): m/z 313 (M+H)⁺

### Example 2

Example 2 presents the preparation of N-(4-methylpyrimidin-2-yl)-6H-[1,3]thiazolo[4,5-e]indazol-2-amine (Table 1, Compound No. 2, identified Cmpd 2)

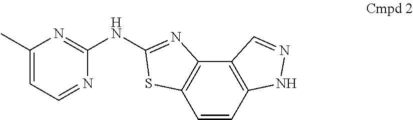 [SMILES: Cc1ccnc(Nc2nc3c(ccc4[nH]ncc43)s2)n1]

### Example 2

### Step A

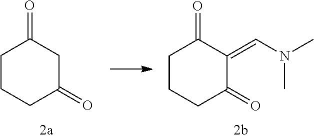

A solution of cyclohexane-1,3-dione (112 g, 1 mol) and 1,1-dimethoxy-N,N-dimethyl methanamine (1.1 mol, 68:2 mL) was stirred at room temperature for 1 hour. After evaporation and trituration with cyclohexane, 2b (137 g, 82%) was obtained as a pale yellow solid.

This compound was characterized by mass spectroscopy (MS) in accordance with the procedure described herein. Results of MS indicated MS (ESI): m/z 168 (M+H)⁺

### Example 2

### Step B

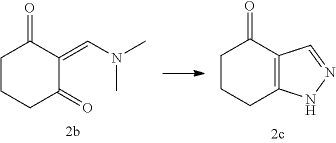

To a solution of 2b (50 g, 299 mmol) in EtOH (500 mL) was added acetic acid (13 mL) followed by hydrazine monohydrate (17.1 mL, 299 mmol), slowly at 0° C. The reaction mixture was then heated to reflux for 17 hrs cooled to r.t., the mixture filtered, and the solvent evaporated to give the title compound 2c (15 g, 36.8%).

This compound was characterized by mass spectroscopy (MS) in accordance with the procedure described herein. Results of MS indicated MS (ESI): m/z 137 (M+H)⁺

### Example 2

### Step C

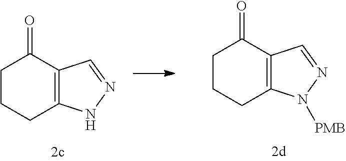

To a solution of 2c (15 g, 110 mmol) in MeCN (300 mL) was added PMBCl (20.6 g, 130 mmol) and K₂CO₃ (30.4 g, 220 mmol) and then the stirred mixture heated to reflux for 5 hrs, then cooled to room temperature. The mixture was filtered and the filtrate concentrated in vacuo to give the crude product, which was purified by chromatography on silica (PE:EtOAc=10:1 to 1:1) to give 2d (20.0 g, 70.9%).

This compound was characterized by proton NMR (¹HNMR) and by mass spectroscopy (MS) in accordance with the procedures described herein. Proton NMR analysis yielded ¹H NMR (CDCl₃ 400 MHz): 7.75 (s, 1H), 7.14 (d, J=8.8 Hz, 2H), 6.89 (t, J=9.2 Hz, 2H), 5.21 (s, 2H), 3.82 (s, 3H), 2.75 (t, J=6.4 Hz, 2H), 2.44-2.49 (m, 2H), 2.11-2.17 (m, 2H). Mass spectroscopy indicated MS (ESI): m/z 257 (M+H)⁺

### Example 2

### Step D

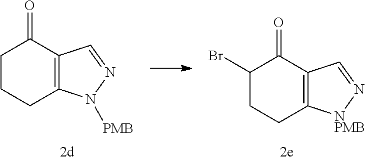

To a solution of 2d (5 g, 19.5 mmol) in chloroform (50 mL) at r.t. was added PhMe₃NBr₃ (7.3 g, 19.5 mmol) and the stirred mixture heated to reflux for 1 hour, and then cooled to room temperature. The mixture was washed with water, the organic phase dried over Na₂SO₄, filtered and the solvent evaporated to afford 2e (5.0 g, 76.9%).

This compound was characterized by mass spectroscopy (MS) in accordance with the procedure described herein. Results of MS indicated MS (ESI): m/z 335, 337 (M+H)⁺

### Example 2

### Step E

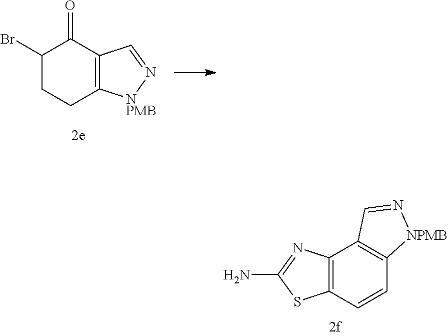

To a mixture of 2e (1 g, 2.9 mmol) and Pd/C (307 mg, 0.29 mmol) in EtOH (15 mL) was added thiourea (1 g, 13.1 mmol). The resulting mixture was heated to reflux for 16 hrs, cooled to room temperature and concentrated in vacuo. EtOAc (20 mL) was added, and the organic phase washed with NaOH (aq), water and brine. The organic layer was dried over Na₂SO₄, filtered and the solvent evaporated to afford the crude product which was purified by chromatography on silica (PE/EtOAc 1:1 to EtOAc) to give 2f (200 mg, 22.1%).

This compound was characterized by proton NMR (¹HNMR) and mass spectroscopy (MS) in accordance with the procedures described herein. Proton NMR yielded the following results: ¹H NMR (CD₃OD, 400 MHz): δ 7.47-7.65 (m, 1H), 7.16 (d, J=8.4 Hz, 1H), 6.82 (d, J=8.8 Hz, 1H). Mass spectroscopy indicated MS (ESI): m/z 311 (M+H)⁺.

### Example 2

### Step F

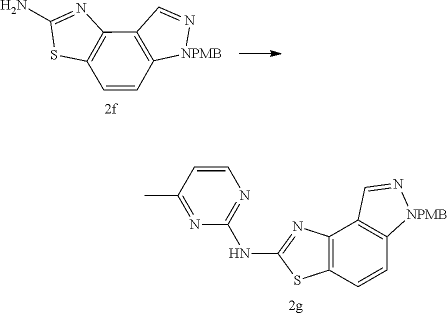

A mixture of compound 2f (150 mg, 0.48 mmol), 2-chloro-4-methylpyrimidine (123 mg, 0.96 mmol), Pd₂(dba)₃ (44 mg, 0.048 mmol), Xant-phos (55 mg, 0.096 mmol) and Cs₂CO₃ (313 mg, 0.96 mmol) in dioxane (8 mL) was heated to reflux for 8 hrs under N₂. The reaction mixture was filtered, concentrated in vacuum and the residue purified by preparative TLC (EtOAc: PE=2:1) to give product 2g (70 mg, 36.3%).

This compound was characterized by mass spectroscopy (MS) in accordance with the procedure described herein. Mass spectroscopy indicated MS (ESI): m/z 403 (M+H)⁺

### Example 2

### Step G

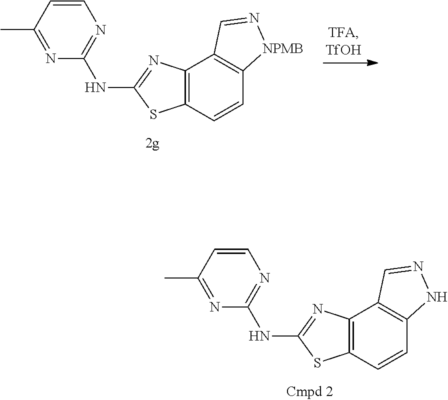

Compound 2g (40 mg, 0.1 mmol) was dissolved in 1 mL of TFA and 1 mL of TfOH. The resulting mixture was stirred and heated at 100° C. under microwave conditions for 30 min. After cooling to r.t., the mixture was concentrated under reduced pressure and the residue purified by preparative HPLC to give the desired compound, with Reference to Table I, above, the Example 2 compound (13 mg, 45.0%).

This compound was characterized by proton NMR (¹HNMR) and mass spectroscopy (MS) in accordance with the procedures described herein. Proton NMR yielded the following results: ¹H NMR (DMSO-d6, 400 MHz): δ 13.23 (s, 1H), 12.01 (s, 1H), δ 8.51 (d, J=4.8 Hz, 2H), 8.20 (s, 1H), 7.83 (d, J=8.8 Hz, 1H), 7.39 (d, J=8.8 Hz, 1H), 6.96 (t, J=5.2 Hz, 1H), 2.46 (s, 3H). Mass spectroscopy indicated MS (ESI): m/z 283 (M+H)⁺.

### Example 3

Example 3 describes preparation of N-(pyrimidin-2-yl)-6H-[1,3]thiazolo[4,5-e]indazol-2-amine (Table I, compound no. 3, identified as Cmpd 3).

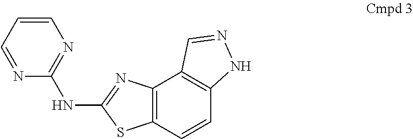 [SMILES: c1cnc(Nc2nc3c(ccc4[nH]ncc43)s2)nc1]

Compound no. 3 of Table 1 (Cmpd 3) was prepared according to the procedure for Example 2, using 2-chloropyrmiidine in place of 2-chloro-4-methylpyrimidine.

This compound was characterized by proton NMR (¹HNMR) and mass spectroscopy (MS) in accordance with the procedures described herein. Proton NMR yielded the following results: ¹H NMR (CD₃OD, 400 MHz): δ 8.57 (d, J=4.8 Hz, 2H), 8.25 (s, 1H), 7.32 (d, J=8.8 Hz, 1H), 7.36 (d, J=8.4 Hz, 1H), 6.97 (t, J=4.8 Hz, 1H). Mass spectroscopy indicated MS (ESI): m/z 269 (M+H)⁺.

### Examples 4 and 5

Compound nos. 4 and 5 of Table I (Cmpd 4 and Cmpd 5) were prepared according to the general procedure for Example 1, using the appropriately substituted chloropyrimidine as indicated in Table III below.

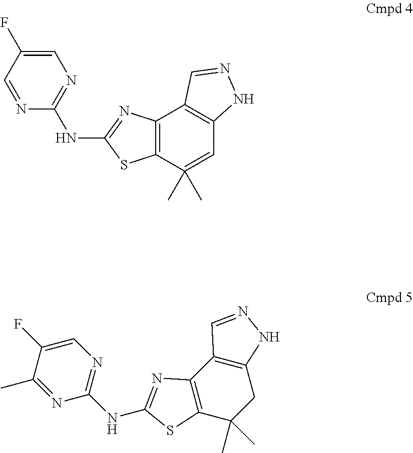

TABLE III

| Cmpd | | MS¹ | |
|---|---|---|---|
| No. | Name | (M + H)⁺ | NMR¹ |
| Cmpd 4 | N-(5-fluoropyrimidin-2-yl)-4,4- | 317 | ¹H NMR ( MeOD 400 MHz ) δ 8.54 |
| | dimethyl-5,6-dihydro-4H- | | (s, 2H), 7.75 (s, 1H), 2.80 (s, 2H), |
| | [1,3]thiazolo[4,5-e]indazol-2-amine | | 1.29 (s, 6H) |
| Cmpd 5 | N-(5-fluoro-4-methylpyrimidin-2-yl)- | 331 | ¹H NMR ( MeOD 400 MHz ) δ 8.34 |
| | 4,4-dimethyl-5,6-dihydro-4H- | | (d, J = 1.6 Hz, 1H), 7.69 (s, 1H), 2.77 |
| | [1,3]thiazolo[4,5-e]indazol-2-amine | | (s, 2H), 2.44 (d, J = 2.4 Hz, 3H), 1.28 |
| | | | (s, 6H). |

¹Data for characterization of compound using Proton NMR and Mass Spectroscopy was obtained in accordance with procedures described herein.

### Example 6

Example 6 presents the preparation of 5-bromo-N-(pyrimidin-2-yl)-6H-[1,3]thiazolo[4,5-e]indazol-2-amine (Table 1, Compound No. 6, identified Cmpd 6)

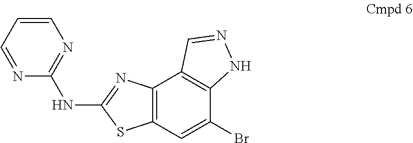 [SMILES: Brc1cc2sc(Nc3ncccn3)nc2c2cn[nH]c12]

### Example 6

### Step A

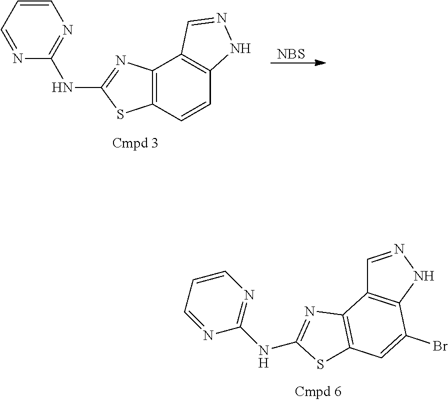

To a stirred solution of Example 3 (26.8 mg, 0.1 mmol) in DMF (1 mL) at 0° C. was added NBS (17.7 mg, 0.1 mmol). The mixture was stirred at 0° C. for 0.5 h, then diluted with H₂O (3 mL) and extracted with EtOAc (10 mL×2). The combined organic layers were dried over Na₂SO₄, filtered and the filtrate was concentrated in vacuum and purified by preparative HPLC to produce title Cmpd 6 (25.4 mg, 73%).

This compound was characterized by proton NMR (¹HNMR) and mass spectroscopy (MS) in accordance with the procedures described herein. Proton NMR yielded the following results: ¹H NMR (DMSO-d6 400 MHz) δ 13.60 (s, 1H), 12.26 (s, 1H), 8.69 (d, J=4.8 Hz, 2H), 8.34 (s, 1H), 8.17 (s, 1H), 7.11 (s, 1H). Mass spectroscopy indicated MS (ESI): m/z 347, 349 (M+H)⁺.

### Example 7

Example 7 presents the preparation of 5-chloro-N-(pyrimidin-2-yl)-6H-[1,3]thiazolo[4,5-e]indazol-2-amine (Table 1, Compound No. 7, identified Cmpd 7)

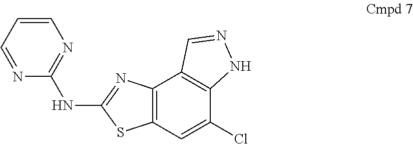 [SMILES: Clc1cc2sc(Nc3ncccn3)nc2c2cn[nH]c12]

Cmpd 7 was prepared using a procedure similar to that described in Example 6, using N-chlorosuccinimide in place of NBS.

This compound was characterized by proton NMR (¹HNMR) and mass spectroscopy (MS) in accordance with the procedures described herein. Proton NMR yielded the following results: ¹H NMR (DMSO-d6 400 MHz) δ 13.73 (s, 1H), 12.26 (s, 1H), 8.68 (d, J=4.8 Hz, 2H), 8.32 (s, 1H), 8.04 (s, 1H), 7.15 (s, 1H). Mass spectroscopy indicated MS (ESI): m/z 303, 305 (M+H)⁺.

### Example 8

Example 8 presents the preparation of 5-methyl-N-(4-methylpyrimidin-2-yl)-6H-[1,3]thiazolo[4,5-e]indazol-2-amine (Table 1, Compound No. 8, identified Cmpd 8)

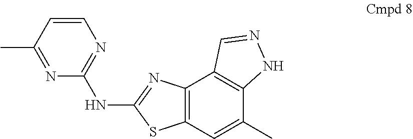

### Example 8

### Step A

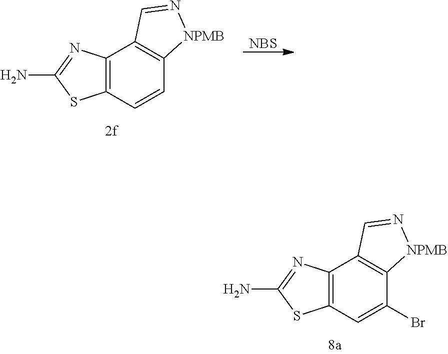

To a stirred solution of 2f (1.55 g, 5 mmol) in DMF (5 mL) at 0° C. was added NBS (885 mg, 5 mmol), the mixture was stirred at 0° C. for 0.5 h, then diluted with H₂O (10 mL) and extracted with EtOAc (20 mL×3). The combined organic layers were dried over Na₂SO₄, filtered and the filtrate was concentrated in vacuo to give crude product 8a (1.55 g, 80%).

This compound was characterized by proton NMR (¹HNMR) and mass spectroscopy (MS) in accordance with the procedures described herein. Proton NMR yielded the following results: ¹H NMR (DMSO-d6 400 MHz) δ 13.73 (s, 1H), 12.26 (s, 1H), 8.68 (d, J=4.8 Hz, 2H), 8.32 (s, 1H), 8.04 (s, 1H), 7.15 (s, 1H). Mass spectroscopy indicated MS (ESI): m/z 389, 391 (M+H)⁺.

### Example 8

### Step B

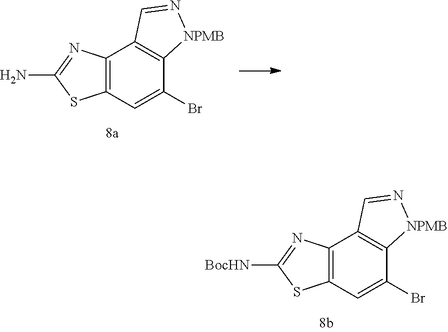

To a stirred solution of 8a (1.55 g, 4 mmol) and DMAP (48.8 mg, 0.4 mmol) in DMF (5 mL) and DCM (10 mL) was added Boc₂O (1.09 g, 5 mmol), the mixture was stirred at room temperature for 15 hrs, then diluted with H₂O (10 mL) and extracted with DCM (20 mL×3). The combined organic layers were dried over Na₂SO₄, filtered and the filtrate was concentrated in vacuo and purified by chromatography on silica to give product 8b (1.53 g, 78%).

This compound was characterized by mass spectroscopy (MS) in accordance with the procedures described herein. Mass spectroscopy indicated MS (ESI): m/z 489, 491 (M+H)⁺.

### Example 8

### Step C

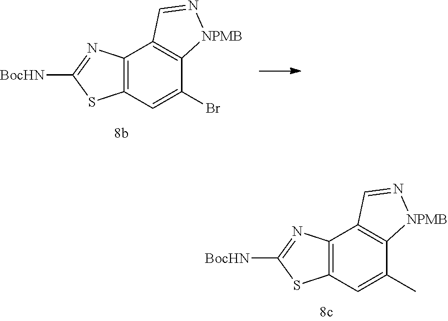

To a mixture of compound 8b (488 mg, 1 mmol) and methylboronic acid (72 mg, 1.2 mmol) in 5 mL of dioxane at r.t. was added Na₂CO₃ (1N, 1 mL) and Pd(dppf)Cl₂ (160 mg, 0.2 mmol) and the mixture heated to 80° C. for 12 hrs under Ar. The reaction mixture was diluted with EtOAc (10 mL), then washed with water (5 mL) and brine (5 mL). The organic layer was dried over anhydrous Na₂SO₄, filtered and the filtrate was concentrated in vacuo and purified by chromatography on silica to give product 8c (353 mg, 83%).

This compound was characterized by mass spectroscopy (MS) in accordance with the procedures described herein. Mass spectroscopy indicated MS (ESI): m/z 425 (M+H)⁺.

### Example 8

### Step D

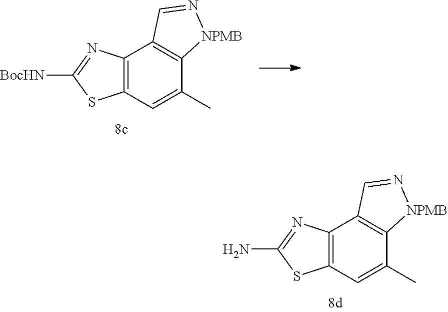

A mixture of compound 8c (353 mg, 0.83 mmol) and TFA (2 mL) was stirred at room temperature for 2 hrs. The reaction mixture was concentrated in vacuo, diluted with EtOAc (10 mL), then washed with 1N NaOH (5 mL) and brine (5 mL). The organic layer was dried over anhydrous Na₂SO₄, filtered and the filtrate was concentrated in vacuo to give product 8d (231 mg, 86%).

This compound was characterized by mass spectroscopy (MS) in accordance with the procedures described herein. Mass spectroscopy indicated MS (ESI): m/z 325 (M+H)⁺.

### Example 8

### Step E

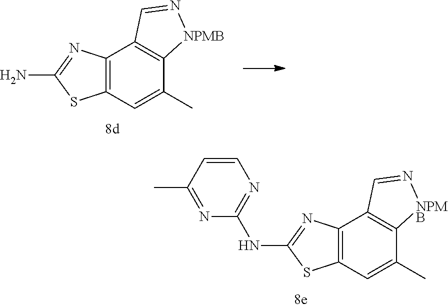

A mixture of compound 8d (162 mg, 0.5 mmol), 2-chloro-4-methylpyrimidine (128 mg, 1 mmol), Pd₂(dba)₃ (30 mg, 0.032 mmol), Xant-phos (37 mg, 0.054 mmol) and Cs₂CO₃ (326 mg, 1 mmol) in dioxane (4 mL) was heated to reflux for 5 hrs under N₂. The mixture was cooled to r.t., filtered and the filtrate concentrated in vacuo. The residue was purified by preparative TLC (EtOAc: PE=1:1) to give product 8e (116 mg, 56%).

This compound was characterized by mass spectroscopy (MS) in accordance with the procedures described herein. Mass spectroscopy indicated MS (ESI): m/z 417 (M+H)⁺.

### Example 8

### Step F

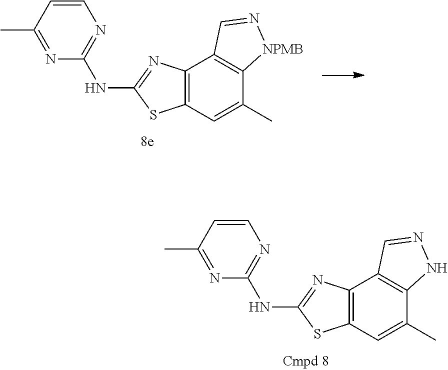

Compound 8e (83.2 mg, 0.2 mmol) was dissolved in 1 mL of TFA and 0.1 mL of TfOH, and the stirred solution heated to 100° C. in a microwave for 20 min. The reaction mixture was cooled to r.t. and concentrated in vacuo. The residue was purified by preparative HPLC to give Example 8 (30.2 mg, 51%).

This compound was characterized by proton NMR (¹HNMR) and mass spectroscopy (MS) in accordance with the procedures described herein. Proton NMR yielded the following results: ¹H NMR (CD₃OD, 400 MHz): δ 8.54 (d, J=4.8 Hz, 1H), 8.25 (s, 1H), 7.65 (s, 1H), 7.00 (d, J=4.8 Hz, 1H), 2.60 (s, 3H), 2.52 (s, 3H). Mass spectroscopy indicated MS (ESI): m/z 297 (M+H)⁺.

### Example 9

Example 9 presents the preparation of 5-methyl-N-(pyrimidin-2-yl)-5,6-dihydro-4H-[1,3]thiazolo[4,5-e]indazol-2-amine (Table 1, Compound No. 9, identified as Cmpd 9)

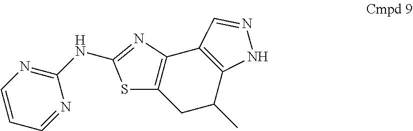 [SMILES: CC1Cc2sc(Nc3ncccn3)nc2-c2cn[nH]c21]

### Example 9

### Step A

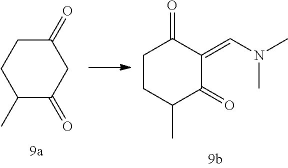

A solution of 4-methylcyclohexane-1,3-dione 9a (1.6 g, 12.6 mmol) and 1,1-dimethoxy-N,N-dimethylmethanamine (13.3 mL, 1.58 mmol) in DCM (10 mL) was stirred at room temperature for 1 hour. After evaporation and trituration in cyclohexane, compound 9b (2.28 g, 78.9%) was obtained as a pale yellow solid.

This compound was characterized by mass spectroscopy (MS) in accordance with the procedures described herein. Mass spectroscopy indicated MS (ESI): m/z 182 (M+H)⁺

### Example 9

### Step B

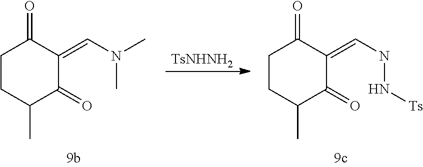 [SMILES: "TsNHNH2" ; CC1CCC(=O)/C(=C/N(C)C)C1=O ; Cc1ccc(S(=O)(=O)NN/C=C2/C(=O)CCC(C)C2=O)cc1]

To a stirred solution of compound 9b (1.8 g, 10.0 mmol) in MeOH (30 mL) was added TsNHNH₂ (2.0 g, 11 mmol) and the mixture was stirred at r.t. for 0.5 h. The reaction mixture was concentrated in vacuo to give compound 9c (2.0 g, 62.5%).

### Example 9

### Step C

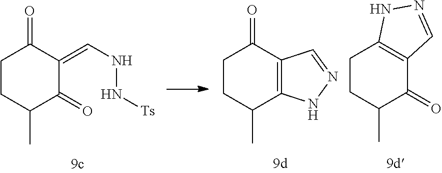

A mixture of compound 9c (1.3 g, 4 mmol), HCl (20 mL, 37% aqueous) and nBuOH (40 mL) was stirred at 110° C. for 1 h. The reaction mixture was concentrated in vacuo to afford crude product 9d and 9d′ (400 mg, 92%) as a mixture which was used directly in the following step.

This compound was characterized by mass spectroscopy (MS) in accordance with the procedures described herein. Mass spectroscopy indicated MS (ESI): m/z 151 (M+H)⁺.

### Example 9

### Step D

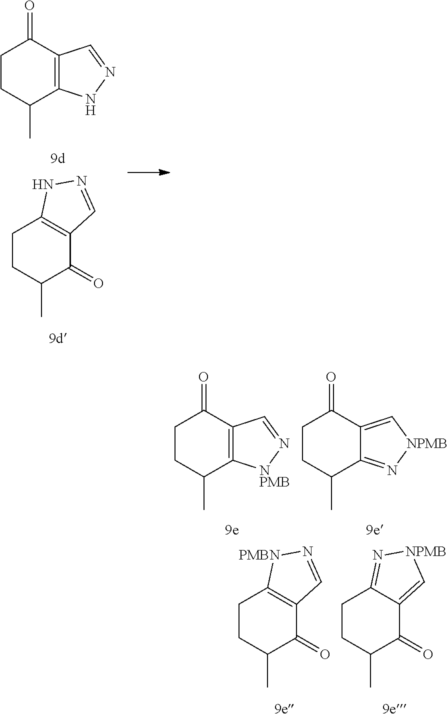

To a mixture of compound 9d and 9d′ (mixture from the preceding step 400 mg, 2.67 mmol) and K₂CO₃ (737 mg, 5.34 mmol) in CH₃CN (15 mL) was added PMBCl (457 mg, 2.93 mmol) and the mixture heated to reflux for 3 h. After cooling to r.t., the mixture was diluted with H₂O (40 mL) and extracted with EtOAc (15 mL×3). The combined organic layers were dried over Na₂SO₄, concentrated in vacuo and purified by preparative TLC to give product (600 mg, 83.2%) as a mixture.

### Example 9

### Step E

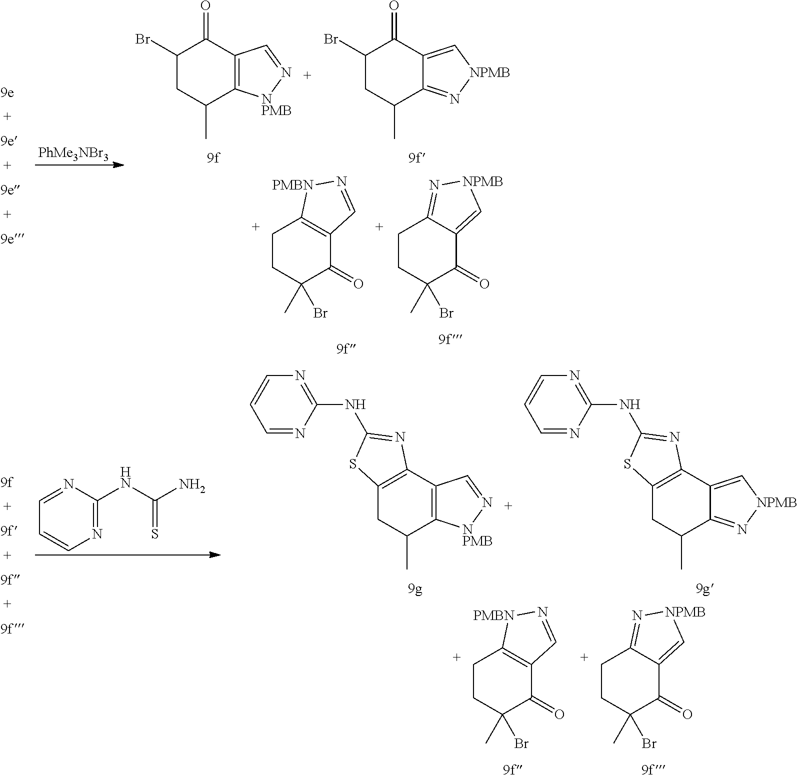

To a solution of compound 9e, 9e′, 9e″ and 9e′″ (600 mg, 2.2 mmol) in chloroform (10 mL) was added trimethylphenylammomium tribromide (835 g, 2.2 mmol, 1 eq.). The reaction mixture was heated to reflux for 1 hour and cooled to r.t. The mixture was diluted with H₂O (20 mL) and extracted with DCM (20 mL×2), the combined organic layers dried over Na₂SO₄ and concentrated in vacuo. 1-Pyrimidin-2-ylthiourea (Rasmussen, C. R et al. Synthesis 1988, 456-9) (88 mg, 0.57 mmol) and EtOH (6 mL) were added to the residue, and the mixture heated to reflux for 5 h. After cooling to r.t., the mixture was concentrated in vacuo, 1N NaOH (10 mL) was added, extracted with EtOAc (10 mL×3). The combined organic layers were washed with H₂O (10 mL) and brine (10 mL), dried over Na₂SO₄, filtered and concentrated in vacuo and purified by preparative TLC to give product 9g and 9g′ as a mixture (40 mg, 17.4%).

This compound was characterized by mass spectroscopy (MS) in accordance with the procedures described herein. Mass spectroscopy indicates MS (ESI): m/z 405 (M+H)⁺.

### Example 9

### Step F

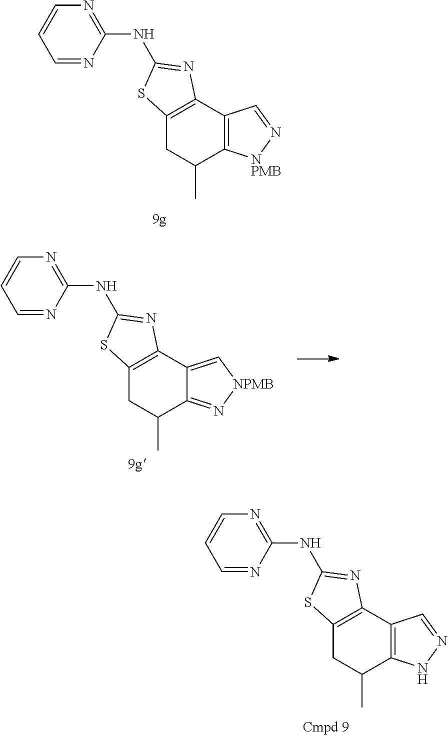

A solution of the mixture of 9g and 9g′ from the preceding step (40 mg, 0.1 mmol) in 2 mL TFA and 0.5 mL water was stirred at 100° C. for 3 h., then cooled to r.t., concentrated in vacuo and purified by preparative HPLC to yield title Cmpd 9 (10 mg, 35.2%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.57 (d, J=4.8 Hz, 2H), 7.59 (s, 1H), 6.97 (t, J=4.8 Hz, 1H), 3.04-3.14 (m, 2H), 2.55-2.63 (m, 1H), 1.29 (d, J=6.8 Hz, 3H). Mass spectroscopy indicated MS (ESI): m/z 285 (M+H)⁺.

### Example 10

Example 10 presents the preparation of 5-methyl-N-(4-methylpyrimidin-2-yl)-5,6-dihydro-4H-[1,3]thiazolo[4,5-e]indazol-2-amine (Table 1, Compound No. 10).

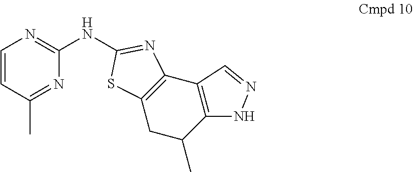 [SMILES: Cc1ccnc(Nc2nc3c(s2)CC(C)c2[nH]ncc2-3)n1]

Cmpd 10 of Example 10 was synthesized according to the procedure for Example 9, replacing 1-pyrimidin-2-ylthiourea with 1-(4-methylpyrimidin-2-yl)thiourea (He, Feng-Qi et al. Chinese Journal of Chemistry (2008), 26(8), 1481-1485).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.41 (d, J=5.2 Hz, 1H), 7.59 (s, 1H), 6.86 (d, J=5.2 Hz, 1H), 3.04-3.14 (m, 2H), 2.55-2.63 (m, 1H), 2.40 (s, 3H), 1.28 (d, J=6.8 Hz, 3H). Mass spectroscopy indicated MS (ESI): m/z 299 (M+H)⁺.

### Example 11

Example 11 presents the preparation of Cmpd 11, N-(4-methylpyrimidin-2-yl)-5′,6′-dihydrospiro[cyclopropane-1,4′-[1,3]thiazolo[4,5-e]indazol]-2′-amine (Table 1, Compound No. 11, identified as Cmpd 11).

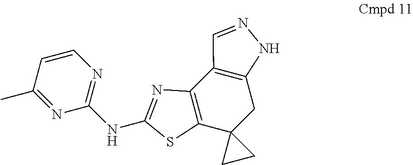 [SMILES: Cc1ccnc(Nc2nc3c(s2)C2(CC2)Cc2[nH]ncc2-3)n1]

### Example 11

### Step A

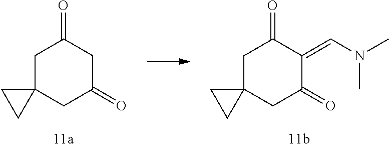

A solution of 11a (0.8 g, 5.8 mmol) and 1,1-dimethoxy-N,N-dimethylmethanamine (0.8 mL, 6 mmol) in DCM (2 mL) was stirred at room temperature for 1 hour. After evaporation and trituration in cyclohexane, 11b (1.08 g, 97%) was obtained as a pale yellow solid.

This compound was characterized by mass spectroscopy (MS) in accordance with the procedures described herein. Mass spectroscopy indicated MS (ESI): m/z 194 (M+H)⁺.

### Example 11

### Step B

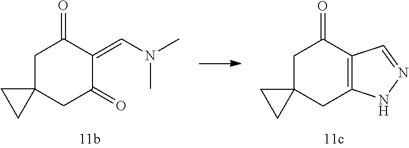

To a stirred solution of compound 11b (1.08 g, 5.6 mmol) in MeOH (10 mL) was added TsNHNH₂ (1.12 g, 6 mmol) and the mixture stirred at room temperature for 1 hour. The mixture was concentrated in vacuo and HCl (2 mL, 37% aqueous) and n-BuOH (2 mL) were added to the residue, the mixture was stirred at 100° C. for 2 h. The mixture was concentrated in vacuo to afford crude product 11c (834 mg, 92%) used directly in the following step.

This compound was characterized by mass spectroscopy (MS) in accordance with the procedures described herein. Mass spectroscopy indicated MS (ESI): m/z 163 (M+H)⁺.

### Example 11

### Step C

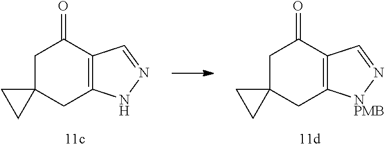

To a mixture of compound 11c (834 mg, 5.1 mmol) and K₂CO₃ (1.38 g, 10 mmol) in CH₃CN (10 mL) was added PMBCl (936 mg, 6 mmol) and the mixture heated to reflux for 5 hrs. After cooling to r.t., the mixture was diluted with H₂O (40 mL) and extracted with EtOAc (10 mL×3), the combined organic layers were dried over Na₂SO₄, filtered and concentrated in vacuo. The residue was purified by chromatography on silica to give product 11d (1.26 g, 88%).

This compound was characterized by mass spectroscopy (MS) in accordance with the procedures described herein. Mass spectroscopy indicated MS (ESI): m/z 283 (M+H)⁺.

### Example 11

### Step D

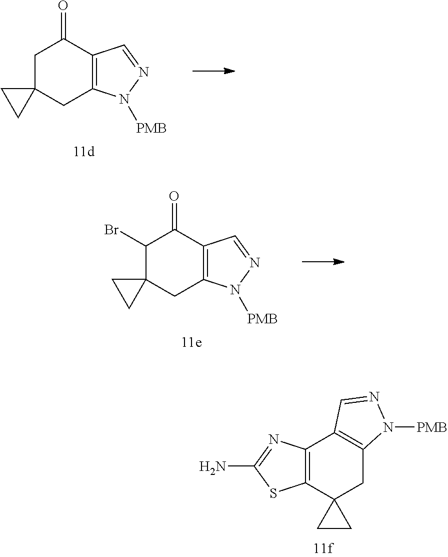

To a solution of ketone 11d (846 mg, 3 mmol) in chloroform (10 mL) was added trimethylphenylammomium tribromide (1.13 g, 3 mmol). The reaction mixture was heated to reflux for 2 hrs., then cooled to rt and diluted with H₂O (20 mL) and extracted with DCM (20 mL×2). The combined organic layers were dried over Na₂SO₄, filtered and the filtrate concentrated in vacuo to give crude product 11e. Thiourea (380 mg, 5 mmol) and EtOH (15 mL) were added to the residue and the mixture heated to reflux for 10 hrs. The mixture was cooled to r.t. and concentrated in vacuo, NaOH (1N, 20 mL) was added to the residue which was extracted with EtOAc (20 mL×3), the combined organic layers were washed with H₂O (10 mL) and brine (10 mL), dried over Na₂SO₄, filtered and the filtrate concentrated in vacuo and purified by chromatography on silica to give product 11f (831 mg, 82%).

This compound was characterized by mass spectroscopy (MS) in accordance with the procedures described herein. Mass spectroscopy indicated MS (ESI): m/z 339 (M+H)⁺.

### Example 11

### Step E

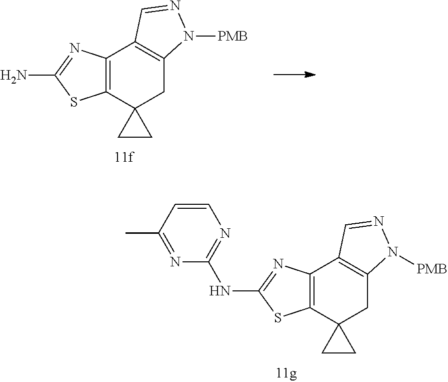

A solution of compound 11f (68 mg, 0.2 mmol), 2-chloro-4-methylpyrimidine (31 mg, 0.24 mmol), Pd₂(dba)₃ (22.85 mg, 0.025 mmol), Xant-phos (28.9 mg, 0.05 mmol) and Cs₂CO₃ (98 mg, 0.3 mmol) in dioxane (3 mL) was heated to reflux for 4 hrs under N₂. After cooling to r.t., the mixture was diluted with H₂O (10 mL) and extracted with EtOAc (10 mL×2), the combined organic layers were dried over Na₂SO₄, concentrated in vacuo and purified by preparative TLC (EtOAc: PE=2:1) to give product 11g (52.4 mg, 62%).

This compound was characterized by mass spectroscopy (MS) in accordance with the procedures described herein. Mass spectroscopy indicated MS (ESI): m/z 431 (M+H)⁺.

### Example 11

### Step F

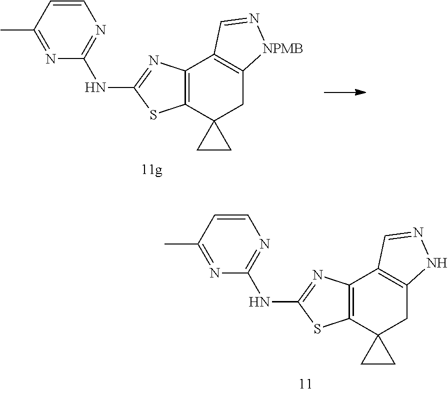

A solution of compound 11g (31.2 mg, 0.075 mmol) in TFA/H₂O (1 mL/0.1 mL) was stirred and heated at 100° C. in a microwave for 0.5 h and cooled to rt. The mixture was concentrated in vacuo and purified by preparative HPLC to yield compound Example 11 (10.5 mg, 47%).

This compound was characterized by proton NMR (¹HNMR) and mass spectroscopy (MS) in accordance with the procedures described herein. Proton NMR yielded the following results: ¹H NMR (CD₃OD, 400 MHz): δ 8.43 (d, J=5.6 Hz, 1H), 7.75 (s, 1H), 6.91 (d, J=5.6 Hz, 1H), 2.92 (s, 2H), 2.55 (s, 3H), 1.01-1.02 (m, 4H). Mass spectroscopy indicated MS (ESI): m/z 311 (M+H)⁺.

### Examples 12-13

Cmpds 12 and 13 (Table I) were prepared according to the general procedure of Example 11. The data obtained from NMR and MS characterization of Cmpds 12 and 13 is reported in Table IV, below.

TABLE IV

| | | | MS¹ | |
|---|---|---|---|---|
| Cmpd. | Structure | Name | (M + H)⁺ | NMR¹ |
| 12 | 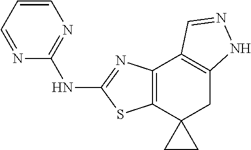 [SMILES: c1cnc(Nc2nc3c(s2)C2(CC2)Cc2[nH]ncc2-3)nc1] | N-(pyrimidin-2-yl)-5′-6′- dihydrospiro[cyclopropane- 1,4′-[1,3]thiazolo[4,5-e] indazol]-2′-amine | 297 | ¹H NMR (MeOD, 400 MHz) δ 8.54 (d, J = 4.8 Hz, 1H), 7.68 (s, 1H), 6.92 (t, J = 4.8 Hz, 1H), 2.84 (s, 2H), 0.91-0.93 (m, 4H). |
| 13 | 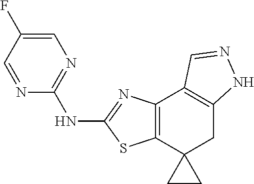 [SMILES: Fc1cnc(Nc2nc3c(s2)C2(CC2)Cc2[nH]ncc2-3)nc1] | N-(5-fluoropyrimidin-2-yl)- 5′,6′-dihydrospiro[cyclo- propane-1,4′-[1,3]thiazolo [4,5-e]indazol]-2′-amine | 315 | ¹H NMR (DMSO-d6 400 MHz): δ 8.68 (s, 2H), 7.63 (s, 1H), 2.83 (s, 2H), 0.85- 0.95 (m, 4H). |

¹Data for characterization of compound using Proton NMR and Mass Spectroscopy was obtained in accordance
with procedures described herein.

### Example 14

Example 14 presents the preparation of Cmpd 14, N-(pyrimidin-2-yl)-1,7-dihydropyrazolo[3′,4′:4,5]cyclopenta[1,2-d][1,3]thiazol-5-amine (Table II, Compound No. 14).

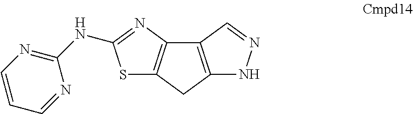 [SMILES: c1cnc(Nc2nc3c(s2)Cc2[nH]ncc2-3)nc1]

### Example 14

### Step A

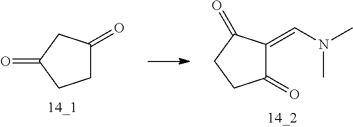

A solution of cyclopentane-1,3-dione (10 g, 0.1 mol) and of 1,1-dimethoxy-N,N-dimethyl methanamine (16 g, 0.12 mol) in 50 mL was stirred at room temperature for 1 hour. After evaporation and trituration in cyclohexane, 14_{—}2 (12 g, 78.4%) was obtained as a pale yellow solid.

This compound was characterized by mass spectroscopy (MS) in accordance with the procedures described herein. Mass spectroscopy indicated MS (ESI): m/z 154 (M+H)⁺.

### Example 14

### Step B

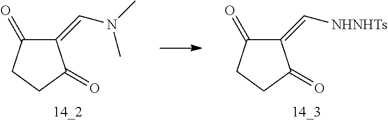

To a stirred solution of compound 14_{—}2 (5.0 g, 32.7 mmol) in MeOH (50 mL) was added TsNHNH₂ (6.4 g, 34.3 mmol) and the mixture stirred at rt for 40 min. The mixture was then concentrated in vacuo to afford compound 14_{—}3 (8.0 g, 83.3%) used in the next step without additional purification.

### Example 14

### Step C

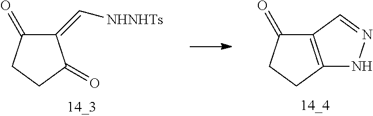

HCl (20 mL, 37% aqueous solution) and nBuOH (60 mL) were added to compound 14_{—}3 (8.0 g, 27.2 mmol) and the mixture stirred at 110° C. for 1 h. After cooling to r.t., the mixture was concentrated in vacuo to give the crude product 14_{—}4 (1.9 g, 57.6%) used in the next step without purification.

This compound was characterized by mass spectroscopy (MS) in accordance with the procedures described herein. Mass spectroscopy indicated MS (ESI): m/z 123 (M+H)⁺.

### Example 14

### Step D

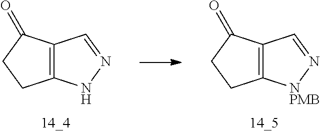

To a mixture of compound 14_{—}4 (1.9 g, 15.6 mmol) and K₂CO₃(4.3 g, 31.2 mmol) in CH₃CN (20 mL) was added PMBCl (2.9 g, 18.7 mmol), and the mixture heated to reflux for 3 h. After cooling to r.t., the mixture was diluted with H₂O (40 mL) and extracted with EtOAc (15 mL×3). The combined organic layers were dried over Na₂SO₄, filtered, concentrated in vacuo and purified by chromatography on silica to give product 14_{—}5 (3.0 g, 80%).

This compound was characterized by mass spectroscopy (MS) in accordance with the procedures described herein. Mass spectroscopy indicated MS (ESI): m/z 243 (M+H)⁺.

### Example 14

### Step E

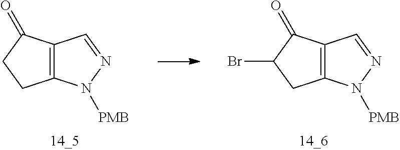

To a solution of compound 14_{—}5 (1.0 g, 4.1 mmol) in chloroform (15 mL) at r.t. was added trimethylphenylammomium tribromide (1.55 g, 4.1 mmol). The reaction mixture was heated to reflux for 1 hour, cooled to r.t., the mixture diluted with H₂O (20 mL) and extracted with DCM (20 mL×2). The combined organic layers were dried over Na₂SO₄ and concentrated in vacuo to give product 146 (1.1 g, 84.6%) used in the next step without purification.

This compound was characterized by mass spectroscopy (MS) in accordance with the procedures described herein. Mass spectroscopy indicated MS (ESI): m/z 323 (M+H)⁺.

### Example 14

### Step F

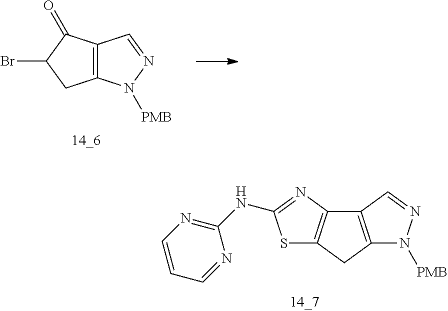

To a solution of compound 14_{—}6 (200 g, 0.63 mmol) in EtOH (6 mL) was added 1-pyrimidin-2-ylthiourea (Rasmussen, C. R et al. Synthesis 1988, 456-9) (96 mg, 0.63 mmol), and the mixture heated to reflux for 5 h. After cooling to r.t., the mixture was filtered and the filtrate concentrated in vacuo to give product 14_{—}7 (100 mg, 42.5%), used without purification in the next step.

This compound was characterized by mass spectroscopy (MS) in accordance with the procedures described herein. Mass spectroscopy indicated MS (ESI): m/z 377 (M+H)⁺.

### Example 14

### Step G

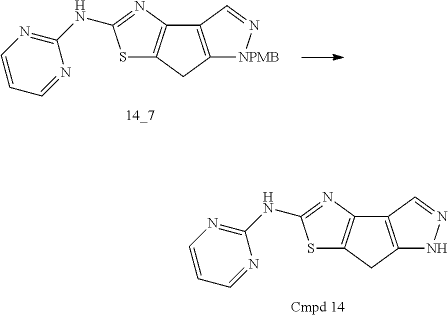

A solution of compound 14_{—}7 (100 mg, 0.26 mmol) in TFA (2 mL) and water (0.5 mL) was stirred at 100° C. for 3 h. The reaction mixture was cooled to r.t., concentrated in vacuo and purified by preparative HPLC to yield Cmpd 14 (22 mg, 33.3%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.60 (d, J=4.8 Hz, 2H), 7.61 (s, 1H), 6.99 (t, J=4.8 Hz, 1H), 3.63 (s, 2H). Mass spectroscopy indicated MS (ESI): m/z 377 (M+H)⁺.

### Example 15

Example 15 presents the preparation of Cmpd 15, N-(5-fluoropyrimidin-2-yl)-6,7-dihydro-4H-pyrazolo[4′,3′:5,6]oxepino[4,3-d][1,3]thiazol-2-amine (Table II, Compound No. 15).

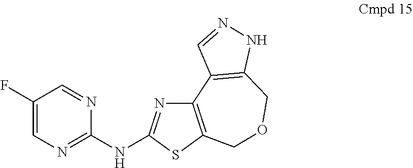 [SMILES: Fc1cnc(Nc2nc3c(s2)COCc2[nH]ncc2-3)nc1]

### Example 15

### Step A

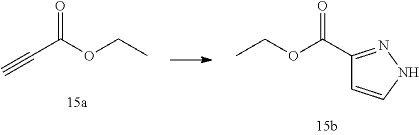

To a solution of ethyl propiolate (30 g, 306 mmol) in THF (200 mL) was added dropwise trimethylsilyldiazomethane (153 mL, 2M in hexanes, 306 mmol) at 20-30° C. with ice bath cooling (delayed exotherm observed). The reaction mixture was stirred for 3 hours at r.t. Water was then added (750 mL) and the organic solvents evaporated. The white precipitate was filtered and dried under vacuum to afford compound 15b (38.5 g, 90%) as a white solid.

### Example 15

### Step B

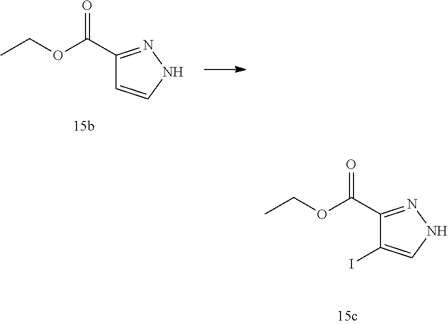

To a solution of compound 15b (38.5 g, 275 mmol) in CH₃CN (700 mL) was added iodine (69.8 g, 275 mmol) followed by ceric ammonium nitrate (150.7 g, 275 mmol). The reaction mixture was then stirred for 12 hours at r.t. Additional iodine (17.4 g) was added and stirring continued for 8 h, following which a cold solution of 5% NaHSO₃ was added to the reaction mixture. The white precipitate was filtered through a celite pad and washed with water and EtOAc. The filtrate layers were separated, the aqueous phase extracted with EtOAc and the organic phases were washed with water, dried over MgSO₄, filtered and solvent evaporated in vacuo to afford compound 15c (38 g, 52%) as a slightly yellow solid.

### Example 15

### Step C

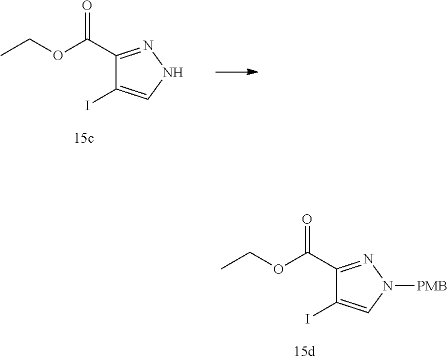

To a solution of compound 15c (38 g, 143 mmol) in acetonitrile (380 mL) at r.t. was added K₂CO₃ (39.5 g, 286 mmol) and then PMBCl (24 mL, 177 mmol). The reaction mixture was stirred at 60° C. overnight. After cooling to r.t., the mixture was filtered and concentrated under reduced pressure. The residue was purified by chromatography on silica gel to afford compound 15d (32 g, 58%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.13 (s, 1H), 7.23 (d, J=8.8 Hz, 2H), 6.90 (d, J=8.8 Hz, 2H), 5.29 (s, 2H), 4.24 (q, J=6.8 Hz, 2H), 3.71 (s, 3H); 1.26 (t, J=6.8 Hz, 3H). Mass spectroscopy indicated MS (ESI): m/z 387 (M+H)⁺.

### Example 15

### Step D

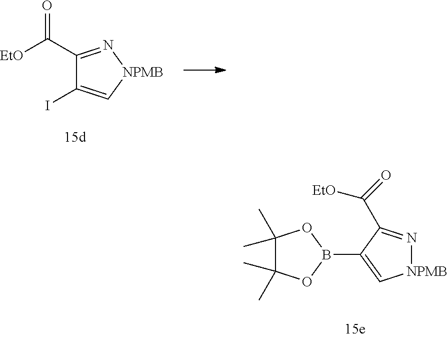

To a solution of 2-isopropoxy-4,4,5,5-tetramethyl-[1,3,2]dioxaborolane (14.5 g, 77.2 mmol) and compound 15d (10 g, 25.9 mmol) in THF (10 mL) at −78° C. was added n-BuLi (30 mL, 2.5 M in hexane, 75 mmol). The reaction mixture was stirred for 2 hrs at −78° C., quenched with water and extracted with EtOAc (2×50 mL). The combined organic layers were dried over MgSO₄, filtered and concentrated in vacuum to give the crude product 15e (10 g, 100%) used directly without purification.

### Example 15

### Step E

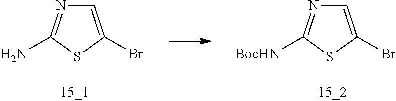 [SMILES: Nc1ncc(Br)s1 ; CC(C)(C)OC(=O)Nc1ncc(Br)s1]

To a solution of 15_{—}1 (58 g, 0.223 mol) in DCM (580 mL) was added Boc₂O (58.3 g, 0.268 mol) and K₂CO₃ (62.0 g, 0.446 mol). The reaction solution was stirred at 20° C. for 16 hours, filtered, concentrated in vacuo and purified by chromatography on silica gel to afford compound 15_{—}2 (40 g, 64%) as a white solid.

### Example 15

### Step F

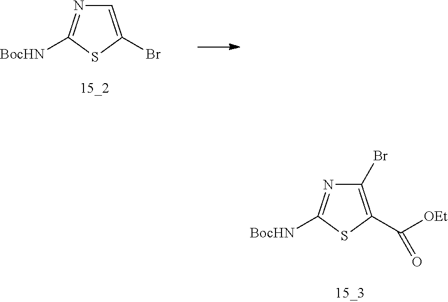 [SMILES: CC(C)(C)OC(=O)Nc1ncc(Br)s1 ; CCOC(=O)c1sc(NC(=O)OC(C)(C)C)nc1Br]

To LDA (177.21 mmol) in THF (100 mL) was added compound 15_{—}2 (15 g, 53.7 mmol) in THF dropwise at 0° C. under N₂ and the mixture stirred at 0° C. for 15 mins. Diethyl pyrocarbonate (28.73 g, 177.21 mmol) was then added dropwise at 0° C. and the reaction mixture stirred at room temperature overnight. The mixture was diluted with EtOAc and washed with water, brine, dried over MgSO₄ and concentrated in vacuo. The residue was purified by silica column chromatography (PE: EtOAc=10:1) to afford product 15_{—}3 (6 g, 32% yield).

This compound was characterized by proton NMR (¹HNMR) in accordance with the procedures described herein. Proton NMR yielded the following results: ¹H NMR (CDCl₃, 400 MHz): δ 9.409 (brs, 1H), 4.367 (q, 2H, J=7.2 Hz), 1.577 (s, 9H), 1.380 (t, 3H, J=7.2 Hz).

### Example 15

### Step G

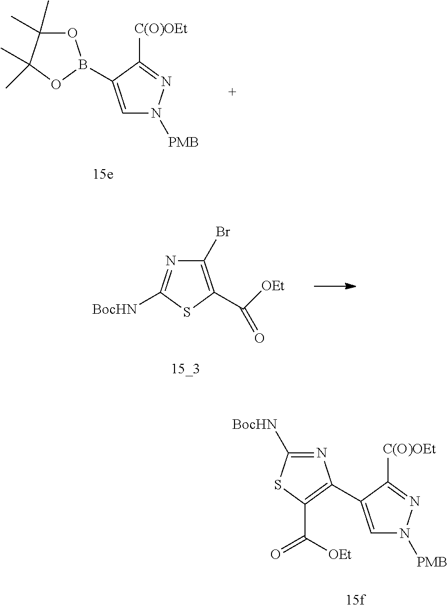

A mixture of 15e (1.1 g, 2.8 mmol), 15_{—}3 (1.0 g, 2.8 mmol), Pd(PPh₃)₂Cl₂ (100 mg), K₃PO₄ (1.2 g, 5.6 mmol) in DMF (8 mL) and water (1.5 mL) was heated to 100° C. for 30 mins. After cooling to r.t., water (20 mL) was added and the mixture was extracted with EtOAc (30 mL×3). The combined organic layers were dried over MgSO₄, filtered and concentrated under reduced pressure. The residue was purified by chromatography on silica to afford compound 15f (0.89 g, 60%).

This compound was characterized by mass spectroscopy (MS) in accordance with the procedures described herein. Mass spectroscopy indicated MS (ESI): m/z 531 (M+H)⁺.

### Example 15

### Step H

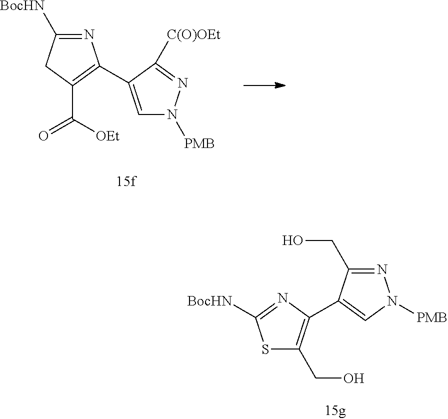

To a solution of compound 15f (0.5 g, 0.94 mmol) in THF (5 mL) at r.t. was added LiBH₄ (2 M, 6 mL) and the mixture heated to reflux for 2 hours. The reaction mixture was cooled to r.t., poured into water and extracted with EtOAc (3×25 mL). The combined organic layers were dried over MgSO₄, filtered and concentrated under reduced pressure. The residue was purified by chromatography on silica to give compound 15g (0.247 g, 59%).

This compound was characterized by mass spectroscopy (MS) in accordance with the procedures described herein. Mass spectroscopy indicated MS (ESI): m/z 447 (M+H)⁺.

### Example 15

### Step I

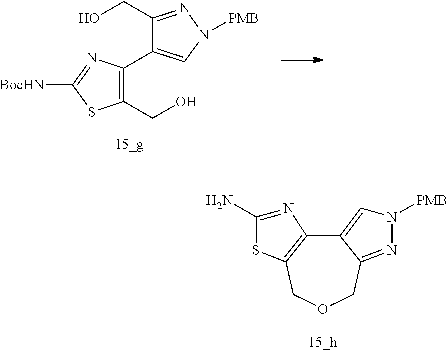

A solution of compound 15g (400 mg, 0.89 mmol) in conc. H₂SO₄ (1 mL) and water (1 mL) was heated to 80° C. for 20 mins. After cooling to r.t., the pH of the reaction mixture was adjusted to 8 by aliquots of 1N NaOH and the mixture extracted with EtOAc (3×25 mL). The combined organic layers were dried over MgSO₄, filtered and concentrated under reduced pressure to give compound 15h (210 mg, 71.9%) used without further purification.

This compound was characterized by proton NMR (¹HNMR) and mass spectroscopy (MS) in accordance with the procedures described herein. Proton NMR yielded the following results: ¹H NMR (CD₃OD, 400 MHz): δ 7.673 (s, 1H), 7.154 (d, 2H, J=8.8 Hz), 6.830 (d, 2H, J=8.8 Hz), 5.112 (s, 2H), 4.839 (s, 2H), 4.780 (s, 2H), 3.690 (s, 3H). Mass spectroscopy indicated MS (ESI): m/z 328 (M+H)⁺.

### Example 15

### Step J

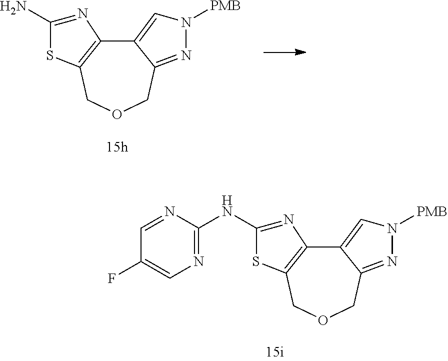

To a solution of compound 15h (50 mg, 0.15 mmol) and 2-chloro-5-fluoropyrimidine (24.2 mg, 0.18 mmol) in dioxane (3 mL) was added Pd₂(dba)₃ (25 mg), Xant-phos (25 mg) and Cs₂CO₃ (100 mg, 0.3 mmol). The reaction mixture was stirred for 2 hours at reflux under nitrogen, cooled to r.t., filtered and the residue washed with EtOAc (30 mL). The filtrate was concentrated under reduced pressure and the residue purified by preparative TLC to give product 15i (30 mg, 46%).

This compound was characterized by mass spectroscopy (MS) in accordance with the procedures described herein. Mass spectroscopy indicated MS (ESI): m/z 425 (M+H)⁺.

### Example 15

### Step K

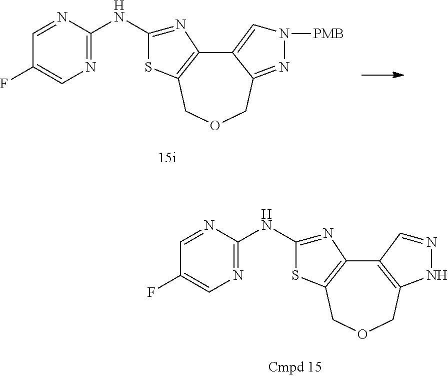

Compound 15i (30 mg, 0.071 mmol) was dissolved in 2 mL of TFA and the solution was stirred at 100° C. in a microwave for 20 mins. The reaction mixture was cooled to r.t., diluted with DMSO (5 mL) and filtered. The filtrate was collected and purified by Prep HPLC to give the product Cmpd 15 as an off-white solid (1.96 mg, 9%).

This compound was characterized by proton NMR (¹HNMR) and mass spectroscopy (MS) in accordance with the procedures described herein. Proton NMR yielded the following results: ¹H NMR (DMSO-d6, 400 MHz): δ 11.80 (s, 1H), 8.68 (s, 2H), 7.74 (s, 1H), 4.96 (s, 2H), 4.93 (s, 2H). Mass spectroscopy indicated MS (ESI): m/z 305 (M+H)⁺.

Examples 16, 17 and 19 illustrate the preparation of Cmpd 16, Cmpd 17 and Cmpd 19, respectively, according to the general procedure for Example 15 by using the appropriately substituted 2-chloropyrimidine in step J. NMR and MS characterization data for compounds Cmpd 16 and Cmpd 17 are reported below in Table V. Activity for these compounds, in the form of EC₅₀ values, are reported in Table II, above.

TABLE V

| | | | MS¹ | |
|---|---|---|---|---|
| Ex. | Structure | | (M + H)⁺ | NMR¹ |
| 16 | 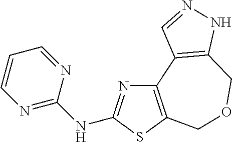 [SMILES: c1cnc(Nc2nc3c(s2)COCc2[nH]ncc2-3)nc1] | N-(pyrimidin-2-yl)-6,7- dihydro-4H-pyrazolo [4′,3′:5,6]oxepino[4,3-d] [1,3]thiazol-2-amine | 287 | ¹H NMR (DMSO-d6, 400 MHz) δ 11.68 (s, 1H), 8.56 (d, 2H, J = 4.8 Hz), 7.77 (s, 1H), 7.01 (t, 1H, J = 4.4 Hz), 4.99 (s, 2H), 4.95 (s, 2H) |
| 17 | 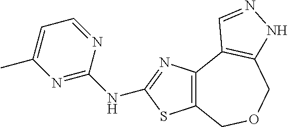 [SMILES: Cc1ccnc(Nc2nc3c(s2)COCc2[nH]ncc2-3)n1] | N-(4-methylpyrimidin- 2-yl)-6,7-dihydro-4H- pyrazolo[4′,3′:5,6] oxepino[4,3-d][1,3] thiazol-2-amine | 301 | ¹H NMR (DMSO-d6, 400 MHz) δ 11.75 (s, 1H), 8.47 (d, 1H, J = 5.2 Hz), 7.94 (s, 1H), 6.94 (d, 1H, J = 5.2 Hz), 4.65 (s, 2H), 4.60 (s, 2H), 2.44 (s, 3H) |
| 19 | 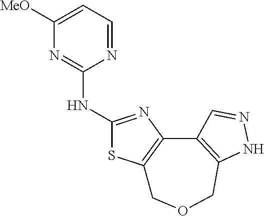 [SMILES: COc1ccnc(Nc2nc3c(s2)COCc2[nH]ncc2-3)n1] | N-(4-methoxypyrimidin- 2-yl)-6,7-dihydro-4H- pyrazolo[4′,3′:5,6] oxepino[4,3-d][1,3] thiazol-2-amine | 317 | ¹H NMR (DMSO-d6, 400 MHz): δ 8.28 (d, 1H, J = 6.0 Hz), 7.82 (s, 1H), 6.45 (d, 1H, J = 6.0 Hz), 4.96 (s, 2H), 4.94 (s, 2H), 4.00 (s, 3H) |

¹Data for characterization of compound using Proton NMR and Mass Spectroscopy was obtained in accordance with
procedures described herein.

### Example 18

Example 18 presents the preparation of Cmpd 18, N-(4-methylpyrimidin-2-yl)-6-methyl-6,7-dihydro-4H-pyrazolo[4′,3′:5,6]oxepino[4,3-d][1,3]-thiazol-2-amine (Table II, Compound No. 18).

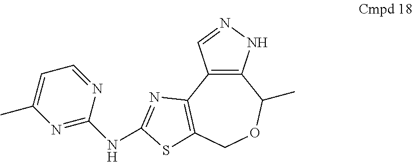

### Example 18

### Step A

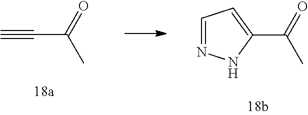

To a solution of but-3-yn-2-one (20.4 g, 300 mmol) in THF (250 mL) was added dropwise trimethylsilyldiazomethane (150 mL, 2M in hexane, 300 mmol) at 20-30° C. with ice bath cooling (delayed exotherm observed). The reaction mixture was stirred for 3 hours at rt. Water was added (500 mL) and the mixture extracted with EtOAc. The combined organic layer was dried over Na₂SO₄, filtered and the filtrate concentrated in vacuo to afford product 18b (31 g, 94%).

### Example 18

### Step B

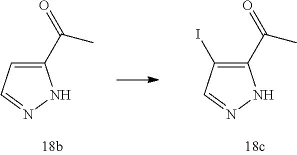

To a solution of compound 18b (31 g, 280 mmol) in CH₃CN (500 mL) was added iodine (38 g, 150 mmol) and then ceric ammonium nitrate (164 g, 300 mmol) and the reaction mixture stirred for 24 hours at r.t. A cold solution of 5% aqueous NaHSO₃ was added to the reaction mixture and the mixture extracted with EtOAc (300 mL×3). The combined organic layers were dried over MgSO₄, filtered and concentrated in vacuo to give product 18c (65 g, 98%) which was used without further purification.

### Example 18

### Step C

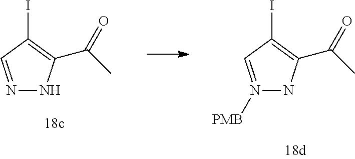

To a solution of compound 18c (65 g, 275 mmol) in acetonitrile (300 mL) was added potassium carbonate (76.0 g, 550 mmol) followed by PMBCl (45.6 g, 290 mmol). The reaction mixture was stirred at 60° C. overnight, cooled to r.t., filtered and the filtrate concentrated in vacuo. The residue was purified by chromatography on silica to give product 18d (88 g, 90%).

This compound was characterized by mass spectroscopy (MS) in accordance with the procedures described herein. Mass spectroscopy indicated MS (ESI): m/z 357 (M+H)⁺.

### Example 18

### Step D

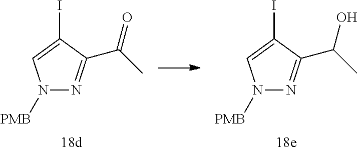

To a solution of compound 18d (17.8 g, 50 mmol) in MeOH (100 mL) at −10° C. was added NaBH₄ (7.6 g, 200 mmol), the mixture stirred at 0° C. for 1 h and the mixture concentrated. Water (20 mL) was added and the mixture extracted with EtOAc (30 mL×3). The combined organic layers were dried over MgSO₄, filtered and concentrated under reduced pressure to give product 18e (17.5 g crude, 98%) which was used directly for the next step without further purification.

This compound was characterized by proton NMR (¹HNMR) and mass spectroscopy (MS) in accordance with the procedures described herein. Proton NMR yielded the following results: ¹H NMR (CDCl₃, 400 MHz) δ 7.26 (s, 1H), 7.18 (d, J=8.4 Hz, 2H), 6.88 (d, J=8.4 Hz, 2H), 5.18 (s, 2H), 4.88-4.92 (m, 1H), 3.80 (s, 3H), 1.56 (d, J=6.4 Hz, 3H). Mass spectroscopy indicated MS (ESI): m/z 359 (M+H)⁺.

### Example 18

### Step E

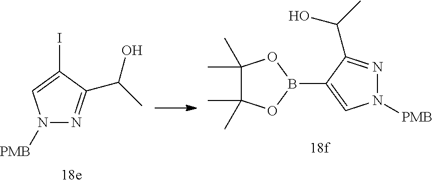

To a solution of 18e (17.5 g, 49 mmol) and 2-isopropoxy-4,4,5,5-tetramethyl-[1,3,2]dioxaborolane (18.6 g, 100 mmol) in THF (100 mL) at −78° C. under N₂, was added CH₃Li (3 M, 50 mL) over 30 mins. The reaction mixture was stirred at −78° C. for 2 h, then quenched with water (50 mL) and extracted with EtOAc (50 mL×3). The combined organic layers were dried over MgSO₄, filtered and concentrated under reduced pressure to give the crude product 18f which was used directly for the next step without further purification (16.8 g crude, 95%).

This compound was characterized by mass spectroscopy (MS) in accordance with the procedures described herein. Mass spectroscopy indicated MS (ESI): m/z 359 (M+H)⁺.

### Example 18

### Step F

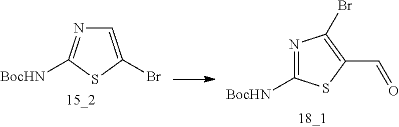 [SMILES: CC(C)(C)OC(=O)Nc1ncc(Br)s1 ; CC(C)(C)OC(=O)Nc1nc(Br)c(C=O)s1]

To (i-Pr)₂NH (59.7 g, 0.591 mol) in THF (250 mL) at 0° C. was added n-BuLi (236 mL, 2.5 M, 0.591 mol) was added slowly. The reaction mixture was stirred for 20 mins after which a THF solution of compound 15_{—}2 (50 g, 0.179 mol) was slowly added to the reaction mixture with continued stirring. The mixture was stirred for 30 mins at 0° C. and then DMF (43.1 g, 0.591 mol) added. The mixture was stirred for 12 hrs at r.t. and diluted with EtOAc and water. The organic layer was separated, washed with brine, dried over Na₂SO₄ and concentrated in vacuo. The residue was purified by silica chromatography to give compound 18_{—}1 (35 g, 64%) as a white solid.

This compound was characterized by proton NMR (¹HNMR) in accordance with the procedures described herein. Proton NMR yielded the following results: ¹H NMR (DMSO-d6, 400 MHz): δ 9.73 (s, 1H); 1.47 (s, 9H).

### Example 18

### Step G

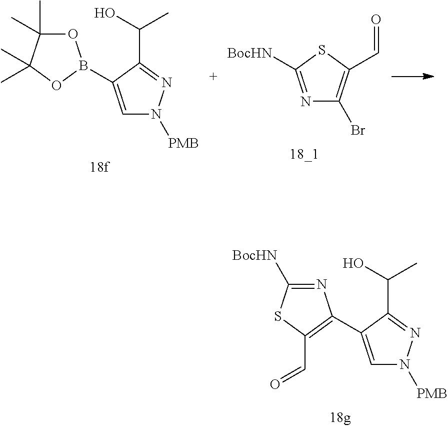

A mixture of 18f (358 mg, 1 mmol), 18_{—}1 (306 mg, 1 mmol), Pd(PPh₃)₂Cl₂ (700 mg), K₃PO₄ (424 mg, 2 mmol) in DMF (5 mL) and water (1 mL) was heated to 100° C. for 30 mins. After cooling to r.t., water (20 mL) was added and the mixture was extracted with EtOAc (30 mL×3). The combined organic layers were dried over MgSO₄, filtered and concentrated under reduced pressure to give crude which was purified by chromatography on silica to afford compound 18g (376 mg, 82%).

This compound was characterized mass spectroscopy (MS) in accordance with the procedures described herein. Mass spectroscopy indicated MS (ESI): m/z 459 (M+H)⁺.

### Example 18

### Step H

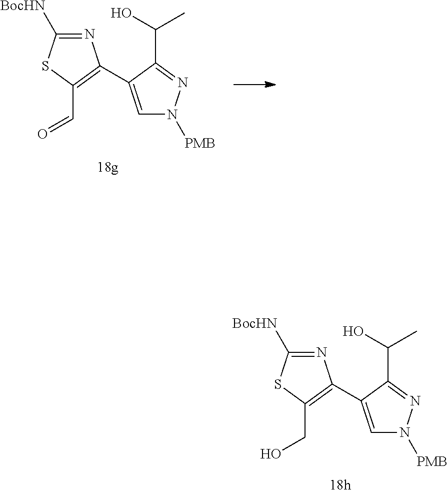

To a stirred solution of compound 18g (275 mg, 0.6 mmol) in MeOH (5 mL) at r.t. was added NaBH₄ (380 mg, 10 mmol) and the mixture stirred for 1 hour. The reaction mixture was poured into cold water and extracted with EtOAc (3×25 mL). The combined organic layers were dried over MgSO₄, filtered and concentrated under reduced pressure to give product 18h (275 mg, 100%), which was used directly in the next step.

This compound was characterized by mass spectroscopy (MS) in accordance with the procedures described herein. Mass spectroscopy indicated MS (ESI): m/z 461 (M+H)⁺.

### Example 18

### Step I

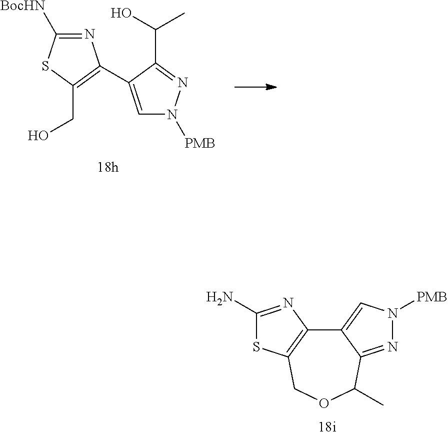

Compound 18h from Step H (275 mg, 0.6 mmol) was added to conc. H₂SO₄ (1 mL) and water (1 mL), and the mixture was heated to 80° C. for 20 mins. After cooling to r.t., aliquots of 1N NaOH were added to adjust the mixture to pH=8, and the mixture extracted with EtOAc (3×25 mL). The combined organic layers were dried over MgSO₄, filtered and concentrated under reduced pressure. The residue was purified by preparative TLC to give product 18i (151 mg, 74%).

This compound was characterized by mass spectroscopy (MS) in accordance with the procedures described herein. Mass spectroscopy indicated MS (ESI): m/z 343 (M+H)⁺.

### Example 18

### Step J

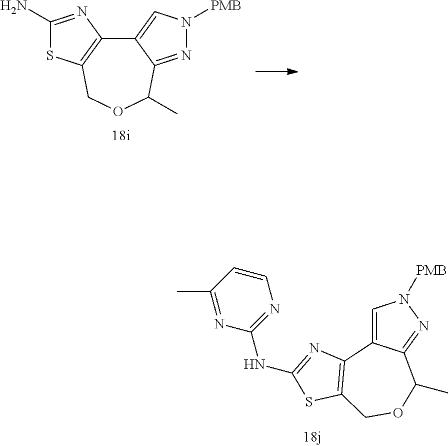

A mixture of compound 18i (68.4 mg, 0.2 mmol) and 2-chloro-4-methylpyrimidine (25.6 mg, 0.2 mmol), Pd₂(dba)₃ (18 mg), Xant-phos (25 mg) and Cs₂CO₃ (100 mg, 0.3 mmol) in dioxane (3 mL) was heated to reflux for 5 h under nitrogen. After cooling to r.t., the mixture was filtered and the residue was washed with EtOAc (30 mL). The filtrate was collected, concentrated under reduced pressure and purified by preparative TLC to give product 18j (52.1 mg, 60%).

This compound was characterized by mass spectroscopy (MS) in accordance with the procedures described herein. Mass spectroscopy indicated MS (ESI): m/z 435 (M+H)⁺.

### Example 18

### Step K

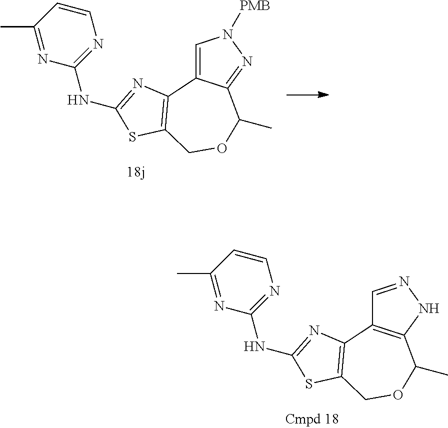

Compound 18j (45 mg, 0.1 mmol) was dissolved in 2 mL of TFA. The mixture was stirred at 100° C. in a microwave for 2 hr. After cooling to r.t., the mixture was concentrated in vacuum and purified by preparative HPLC to afford Example 18 (5 mg, 16%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.42 (d, J=4.8 Hz, 1H), 7.75 (s, 1H), 6.88 (d, J=4.8 Hz, 1H), 4.93-5.06 (m, 2H), 4.87-4.91 (m, 1H), 2.41 (s, 3H), 1.53 (d, J=6.4 Hz, 3H). Mass spectroscopy indicated MS (ESI): m/z 315 (M+H)⁺.

### Examples 19a-22

Using the procedures of Example 18, analogous compounds, Cmpd 19a (6-Methyl-N-[4-(propan-2-yl)pyrimidin-2-yl]-6,7-dihydro-4H-pyrazolo[4′,3′:5,6]oxepino[4,3-d][1,3]thiazol-2-amine), Cmpd 20 (6-Methyl-N-[4-(ethyl)pyrimidin-2-yl]-6,7-dihydro-4H-pyrazolo[4′,3′:5,6]oxepino[4,3-d][1,3]thiazol-2-amine), Cmpd 20a (N-(4-Cyclopropylpyrimidin-2-yl)-6-methyl-6,7-dihydro-4H-pyrazolo[4′,3′:5,6]oxepino[4,3-d][1,3]thiazol-2-amine), Cmpd 21 (N-(5-Fluoro-4-methylpyrimidin-2-yl)-6-methyl-6,7-dihydro-4H-pyrazolo[4′,3′:5,6]oxepino[4,3-d][1,3]thiazol-2-amine), and Cmpd 22 (N-(5-Fluoropyrimidin-2-yl)-6-methyl-6,7-dihydro-4H-pyrazolo[4′,3′:5,6]oxepino[4,3-d][1,3]thiazol-2-amine) can be prepared by substituting 2-chloro-4-methylpyrimidine with the appropriately substituted 2-chloropyrimidine. These compounds were prepared and characterized by proton NMR (¹HNMR) and/or mass spectroscopy (MS) in accordance with the procedures described herein. The chemical structure and for each compound analyzed, the data from Proton NMR and/or MS analysis, is listed in Table VI.

TABLE VI

| Cmpd. | | MS¹ | |
|---|---|---|---|
| No. | Structure | MS (M + H)⁺ | NMR¹ |
| 19a | 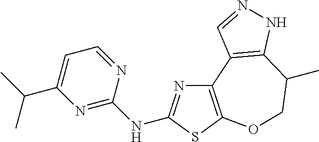 [SMILES: CC(C)c1ccnc(Nc2nc3c(s2)OCC(C)c2[nH]ncc2-3)n1] | 343 | ¹H NMR (DMSO-d6, 400 MHz): δ 8.44 (d, 1H, J = 5.2 Hz), 7.75 (s, 1H), 6.89 (d, 1H, J = 5.2 Hz), 5.03 (d, 1H, J = 16.0 Hz), 4.98 (q, 1H, J = 6.4 Hz), 4.88 (d, 1H, J = 16.0 Hz), 2.90-2.97 (m, 1H), 1.52 (d, 3H, J = 6.4 Hz), 1.25 (d, 6H, J = 6.4 Hz). |
| 20 | 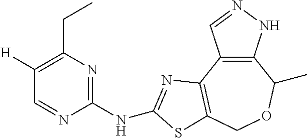 [SMILES: CCc1ccnc(Nc2nc3c(s2)COC(C)c2[nH]ncc2-3)n1] | 329 | ¹H NMR (DMSO-d6, 400 MHz): δ 8.44 (d, 1H, J = 5.2 Hz), 7.75 (s, 1H), 6.88 (d, 1H, J = 5.2 Hz), 5.03 (d, 1H, J = 16.0 Hz), 4.98 (q, 1H, J = 6.8 Hz), 4.87 (d, 1H, J = 16.0 Hz), 2.69 (q, 2H, J = 7.6 Hz), 1.52 (d, 3H, J = 6.8 Hz), 1.25 (t, 3H, J = 7.6 Hz). |
| 20a | 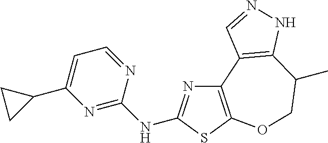 [SMILES: CC1COc2sc(Nc3nccc(C4CC4)n3)nc2-c2cn[nH]c21] | 341 | ¹H NMR (DMSO-d6, 400 MHz): δ 8.32 (d, 1H, J = 4.8 Hz), 7.75 (s, 1H), 6.94 (d, 1H, J = 5.2 Hz), 5.01 (d, 1H, J = 15.6 Hz), 4.97 (q, 1H, J = 6.4 Hz), 4.87 (d, 1H, J = 15.6 Hz), 2.05-2.06 (m, 1H), 1.52 (d, 3H, J = 6.4 Hz), 1.07-1.19 (m, 4H). |
| 21 | 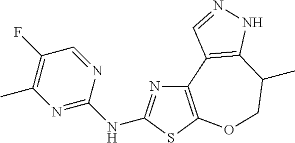 | 333 | ¹H NMR (DMSO-d6, 400 MHz): δ 8.51 (d, 1H, J = 1.2 Hz), 7.74 (s, 1H), 5.03 (d, 1H, J = 16.0 Hz), 4.98 (q, 1H, J = 6.4 Hz), 4.87 (d, 1H, J = 16.0 Hz), 2.41 (d, 3H, J = 2.0 Hz), 1.52 (d, 3H, J = 6.4 Hz). |
| 22 | 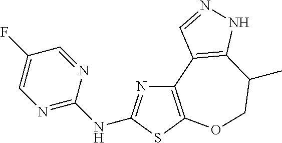 [SMILES: CC1COc2sc(Nc3ncc(F)cn3)nc2-c2cn[nH]c21] | 318 | |

¹NMR and MS data were acquired using procedures described elsewhere herein.

### Example 23

Example 23 presents the preparation of Cmpd 23, 4-methyl-N-(4-methylpyrimidin-2-yl)-6,7-dihydro-4H-pyrazolo[4′,3′:5,6]oxepino[4,3-d][1,3]thiazol-2-amine (Table II, Compound No. 23).

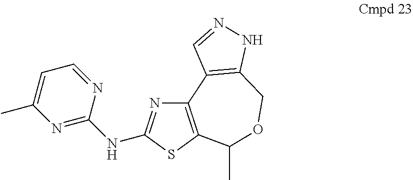 [SMILES: Cc1ccnc(Nc2nc3c(s2)C(C)OCc2[nH]ncc2-3)n1]

### Example 23

### Step A

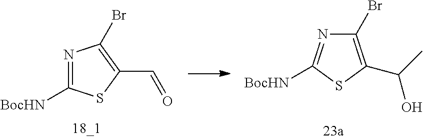 [SMILES: CC(C)(C)OC(=O)Nc1nc(Br)c(C=O)s1 ; CC(O)c1sc(NC(=O)OC(C)(C)C)nc1Br]

To a solution of compound 18_{—}1 (20 g, 65.1 mmol) in THF (200 mL) at −78° C. was added MeMgBr (54.3 mL, 162.7 mmol). The resulting mixture was stirred for 20 mins at −78° C. and allowed to warm up to r.t. for 2 hr. The mixture was quenched with HCl (1M) and extracted with EtOAc. The combined organic layers were dried over MgSO₄, filtered and concentrated under reduced pressure. The residue was purified by chromatography column on silica gel (PE: EtOAc=5:1) to give compound 23a (17 g, 80.9%) as a white solid.

This compound was characterized by mass spectroscopy (MS) in accordance with the procedures described herein. Mass spectroscopy indicated MS (ESI): m/z 323, 325 (M+H)⁺.

### Example 23

### Step B

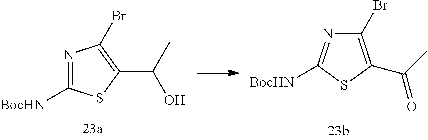 [SMILES: CC(O)c1sc(NC(=O)OC(C)(C)C)nc1Br ; CC(=O)c1sc(NC(=O)OC(C)(C)C)nc1Br]

A mixture of compound 23a (17 g, 52.6 mmol) and PCC (17 g, 78.9 mmol) in DCM (170 mL) was stirred at 20° C. for 16 hrs. Then the mixture was filtered and the solvent removed under reduced pressure to give a brown solid which was purified by column chromatography on silica gel (PE:EtOAc=5:1) to give compound 23b (4.3 g, 25%) as a yellow solid.

This compound was characterized by proton NMR (¹HNMR) and mass spectroscopy (MS) in accordance with the procedures described herein. Proton NMR yielded the following results: ¹H NMR (CDCl₃, 400 MHz): δ 2.64 (s, 3H), 1.54 (s, 9H). Mass spectroscopy indicated MS (ESI): m/z 321, 323 (M+H)⁺.

### Example 23

### Step C

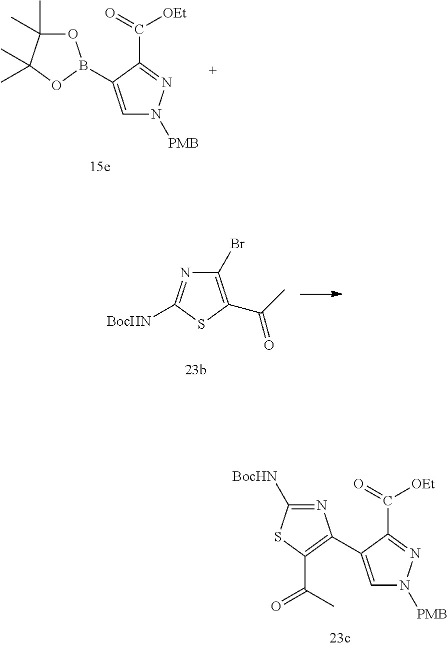

A mixture of compound 23b (3.5 g, 10.9 mmol), 15e (8.0 g, 20.7 mmol), K₃PO₄ (5.8 g, 21.8 mmol) and (PPh₃)₂PdCl₂ (350 mg, 0.5 mmol) in DMF (35 ml) and H₂O (7 mL) was heated to 80° C. for 1.5 hrs under N₂. The mixture was cooled to r.t. and diluted with water and then extracted with EtOAc. The organic layer was washed with brine, dried over Na₂SO₄ and concentrated in vacuo. The residue was purified by chromatography column on silica gel (PE:EA=5:1 to 1:1) to give compound 23c (3.5 g, 64%) as a white solid.

This compound was characterized by proton NMR (¹HNMR) and mass spectroscopy (MS) in accordance with the procedures described herein. Proton NMR yielded the following results: ¹H NMR (CDCl₃, 400 MHz): δ 8.22 (s, 1H), 7.48 (s, 1H), 7.25 (t, J=8.8 Hz, 2H), 6.87 (d, J=8.8 Hz, 2H), 5.34 (s, 2H), 4.26-4.31 (m, 2H), 3.79 (s, 3H), 2.12 (s, 3H), 1.52 (s, 9H), 1.25 (t, J=7.2 Hz, 3H). Mass spectroscopy indicated MS (ESI): m/z 501 (M+H)⁺.

### Example 23

### Step D

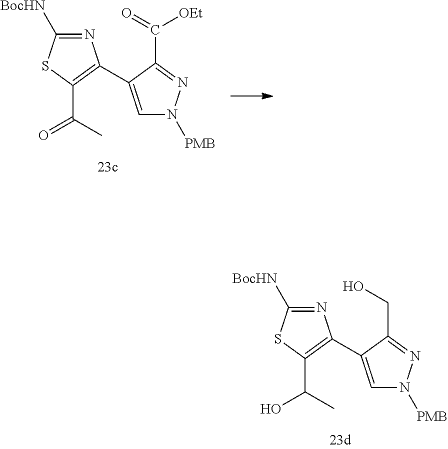

To a solution of compound 23c (500 mg, 1 mmol) in THF was added LiBH₄ (2.5 ml, 5 mmol) in portions. Subsequently the mixture was heated to reflux and reflux was maintained for 2 hrs. Subsequently the mixture was cooled to r.t., quenched by adding water dropwise, and then extracted with EtOAc. The organic layer was separated, washed with brine, dried over sodium sulfate, filtered and concentrated to give the product, compound 23d (300 mg, 65%). The product was used without further purification.

This compound was characterized by mass spectroscopy (MS) in accordance with the procedures described herein. Mass spectroscopy indicated MS (ESI): m/z 461 (M+H)⁺.

### Example 23

### Step E

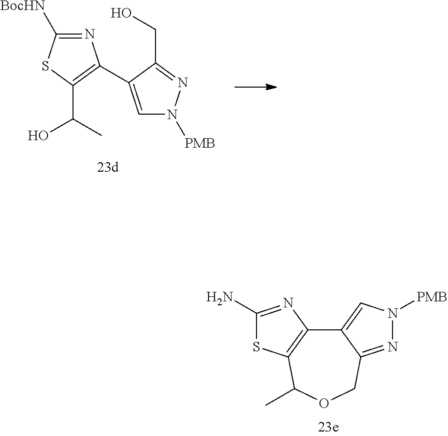

Compound 23d from the previous step (used as prepared, 300 mg, 0.652 mmol), H₂SO₄ (2 mL) and H₂O (2 mL) were heated together to 80° C. for 15 mins. Subsequently the mixture was cooled to r.t., and the pH of the mixture was adjusted to pH=8 by adding 1N NaOH dropwise. Subsequently the mixture was extracted with EtOAc. The organic layer was separated, washed with brine, dried over sodium sulfate, filtered and concentrated under reduced pressure. The residue was purified by chromatography column on silica gel (PE:EtOAc=1:5) to give compound 23e (150 mg, 67%) as a white solid.

This compound was characterized by mass spectroscopy (MS) in accordance with the procedures described herein. Mass spectroscopy indicated MS (ESI): m/z 343 (M+H)⁺.

### Example 23

### Step F

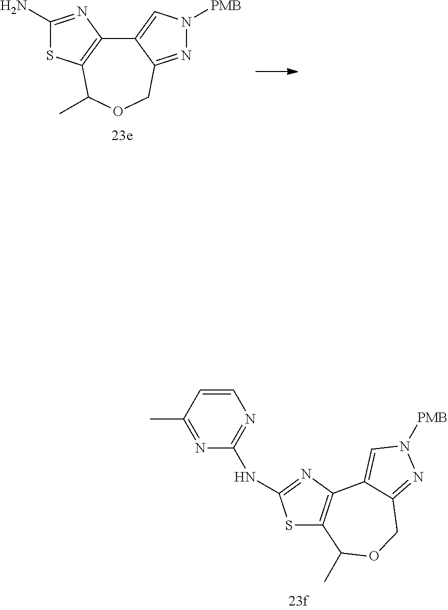

A mixture of compound 23e (100 mg, 0.292 mmol), 2-chloro-4-methyl-pyrimidine (66.7 mg, 0.585 mmol), Pd₂(dba)₃ (13:3 mg, 0.0146 mmol), Xant-Phos (16.9 mg, 0.0292 mmol) and Cs₂CO₃ (190.7 mg, 0.585 mmol) in dioxane (5 mL) was heated to 100° C. for 2 hrs. After cooling to r.t., the mixture was filtered and the filtrate concentrated under reduced pressure. The residue was purified by preparative TLC (PE:EtOAc=1:5) to give product 23f (80 mg, 63%).

This compound was characterized by mass spectroscopy (MS) in accordance with the procedures described herein. Mass spectroscopy indicated MS (ESI): m/z 435 (M+H)⁺.

### Example 23

### Step G

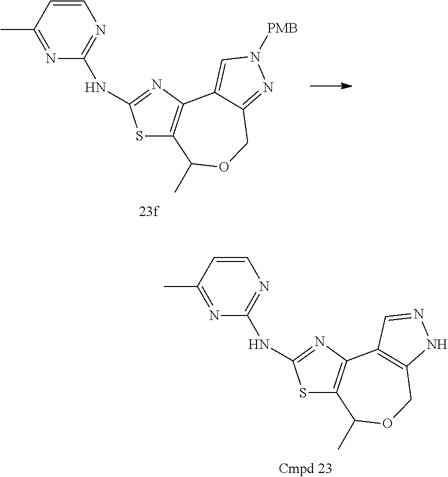

Compound 23f (80 mg, 0.184 mmol) in TFA (2 mL) and H₂O (2 drops) was heated at 100° C. for 20 mins in a microwave oven. The mixture was subsequently cooled to r.t., the solvent was evaporated under reduced pressure and the crude product thus obtained was purified by preparative HPLC to provide the desired product Cmpd 23 (10 mg, 16.2%).

This compound was characterized by proton NMR (¹HNMR) and mass spectroscopy (MS) in accordance with the procedures described herein. Proton NMR yielded the following results: ¹H NMR (DMSO-d6, 400 MHz): δ 11.54 (d, J=3.2 Hz, 1H), 8.45 (d, J=4.8 Hz, 1H), 7.76 (s, 1H), 6.89 (d, J=5.2 Hz, 1H), 5.05-5.10 (m, 1H), 4.90-5.02 (m, 2H), 2.41 (s, 3H), 1.55 (d, J=6.8 Hz, 3H). Mass spectroscopy indicated MS (ESI): m/z 315 (M+H)⁺.

### Example 24

Example 24 presents the preparation of Cmpd 24, 4,6-dimethyl-N-(4-methylpyrimidin-2-yl)-6,7-dihydro-4H-pyrazolo[4′,3′:5,6]oxepino-[4,3-d][1,3]thiazol-2-amine (Table II, Compound No. 24).

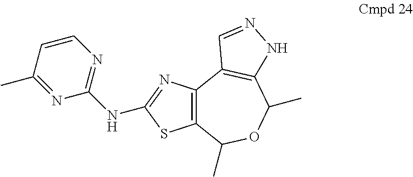

### Example 24

### Step A

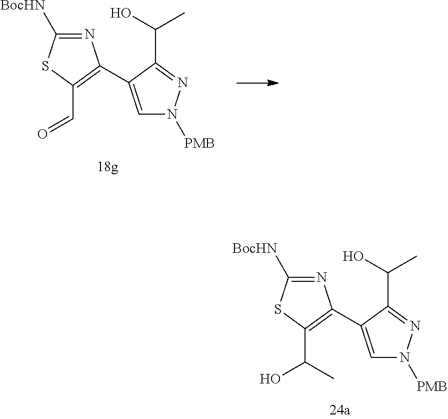

To a stirred solution of compound 18g (458 mg, 1 mmol) in THF (5 mL) was added MeLi (1 mL, 3 mmol) at 0° C. and then the mixture was stirred for one additional hour. The mixture was poured into cooled water (5 mL) and extracted with EtOAc. The combined organic layers were dried over MgSO₄, filtered and concentrated under reduced pressure to give product 24a (458 mg, 100%).

This compound was characterized by mass spectroscopy (MS) in accordance with the procedures described herein. Mass spectroscopy indicated MS (ESI): m/z 475 (M+H)⁺.

### Example 24

### Step B

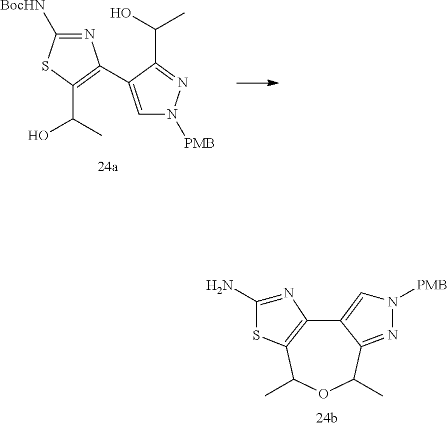

A solution of compound 24a (458 mg, 1 mmol) in conc. H₂SO₄ (1 mL) and water (1 mL) was heated to 80° C. for 20 mins. After cooling, the mixture was brought to pH=8 with aliquots of 1N NaOH and then extracted with EtOAc (3×25 mL). The combined organic layers were dried over MgSO₄, filtered and concentrated under reduced pressure. The residue was purified by preparative TLC (PE:EtOAc=1:1) to give product 24b (212 mg, 59%).

This compound was characterized by mass spectroscopy (MS) in accordance with the procedures described herein. Mass spectroscopy indicated MS (ESI): m/z 357 (M+H)⁺.

### Example 24

### Step C

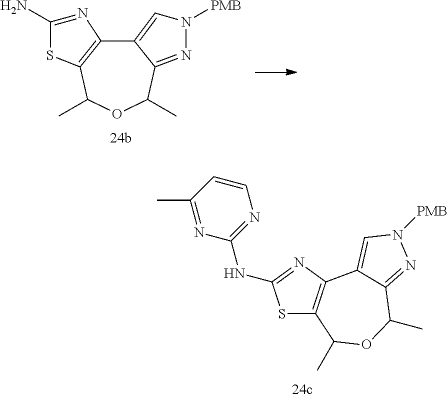

A mixture of compound 24b (71.2 mg, 0.2 mmol), 2-Chloro-4-methyl-pyrimidine (30.7 mg, 0.24 mmol), Pd₂(dba)₃ (18 mg), Xant-phos (25 mg) and Cs₂CO₃ (100 mg, 0.3 mmol) in dioxane (3 mL) was refluxed for 5 h under nitrogen. After cooling, the mixture was filtered and the residue was washed with EtOAc (30 mL). The filtrated was collected and concentrated under reduced pressure and purified by prep. TLC to give the desired product, Cmpd 24 (62 mg, 69%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.41 (d, J=5.2 Hz, 1H), 7.59 (s, 1H), 6.86 (d, J=5.2 Hz, 1H), 3.04-3.14 (m, 2H), 2.55-2.63 (m, 1H), 2.40 (s, 3H), 1.28 (d, J=6.8 Hz, 3H). Mass spectroscopy indicated MS (ESI): m/z 449 (M+H)⁺.

### Example 24

### Step D

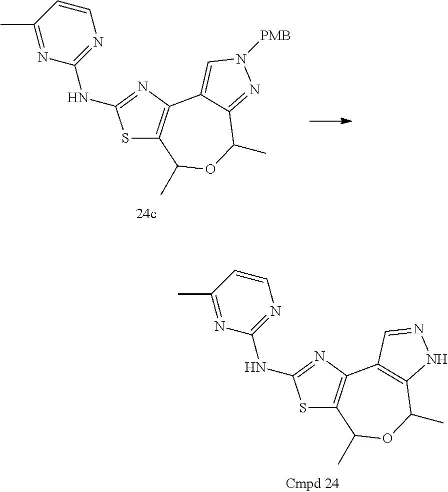

Compound 24c (35 mg, 0.08 mmol) was stirred at 100° C. in a microwave oven in 2 mL of TFA. The mixture was cooled to r.t. then concentrated under reduced pressure and purified by preparative HPLC to give the desired compound, Cmpd 24, (9 mg, 35.1%).

This compound was characterized by proton NMR (¹HNMR) and mass spectroscopy (MS) in accordance with the procedures described herein. Proton NMR yielded the following results: ¹H NMR (DMSO-d6, 400 MHz): 8.44 (d, J=4.8 Hz, 1H), 7.75 (s, 1H), 6.88 (d, J=4.8 Hz, 1H), 5.05-5.11 (m, 2H), 2.41 (s, 3H), 1.51-1.55 (m, 6H). Mass spectroscopy indicated MS (ESI): m/z 329 (M+H)⁺.

### Example 27

Example 27 presents the preparation of Cmpd 27, N-(pyrimidin-2-yl)-4,5,6,7-tetrahydropyrazolo[3,4-c][1,3]thiazolo[4,5-e]azepin-2-amine (Table II, Compound No. 27)

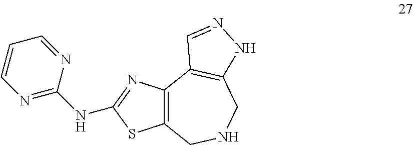 [SMILES: c1cnc(Nc2nc3c(s2)CNCc2[nH]ncc2-3)nc1]

### Example 27

### Step A

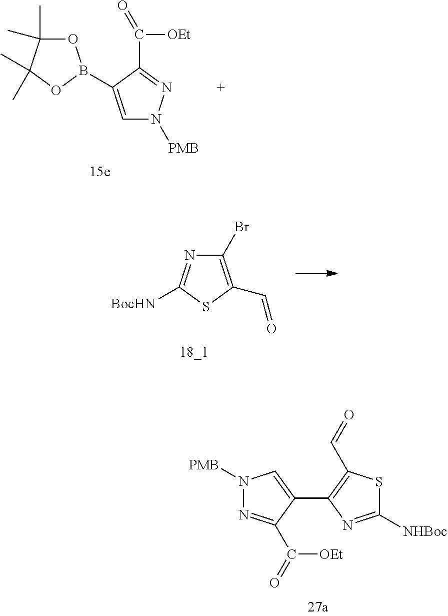

A mixture of 18_{—}1 (2.6 g, 8.46 mmol), 15e (3.27 g, 8.46 mmol), Pd(PPh₃)₄ (500 mg), K₃PO₄ (3.59 g, 16.9 mmol) in DMF (30 mL) and water (6 mL) was heated to 100° C. for 30 mins. After cooling to r.t., water (30 mL) was added and the mixture was extracted with EtOAc (3×25 mL). The combined organic layers were dried over MgSO₄, filtered and concentrated under reduced pressure. The residue was purified by chromatography on silica to give compound 27a (1.6 g, 39%).

This compound was characterized by mass spectroscopy (MS) in accordance with the procedures described herein. Mass spectroscopy indicated MS (ESI): m/z 487 (M+H)⁺.

### Example 27

### Step B

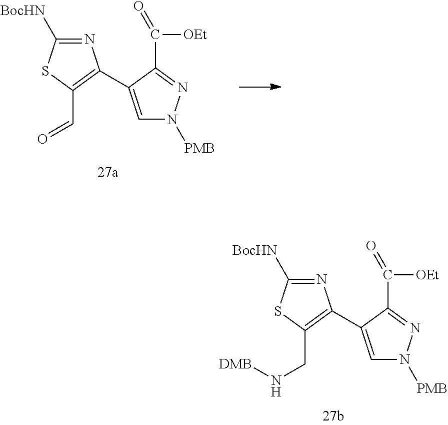

A mixture of compound 27a (1.6 g, 3.3 mmol), 2,4-dimethoxybenzylamine (0.68 g, 3.4 mmol), and 4 A molecular sieves (3 g) in toluene (50 mL) and EtOH (10 mL) was heated to reflux for 18 h. After cooling to r.t., the reaction mixture was filtered and the filtrate concentrated in vacuo. The residue was dissolved in MeOH (10 mL) and NaBH₄ (0.16 g, 4.2 mmol) added at 0° C. and the mixture was stirred for 10 mins. The mixture was poured into water and extracted with EtOAc (3×25 mL). The combined organic layers were dried over MgSO₄, filtered and concentrated under reduced pressure to give the crude 27b (1.4 g crude, 71%) without further purification.

This compound was characterized by mass spectroscopy (MS) in accordance with the procedures described herein. Mass spectroscopy indicated MS (ESI): m/z 638 (M+H)⁺.

### Example 27

### Step C

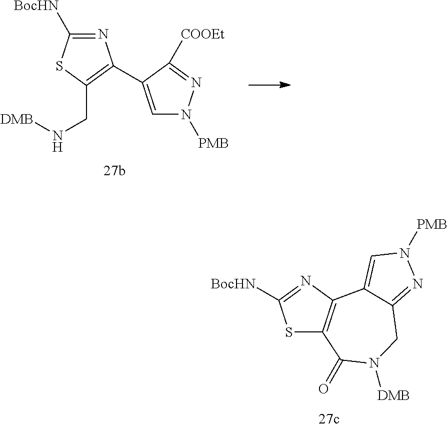

To a solution of compound 27b (1.4 g, 2.24 mmol) in MeOH (20 mL) was added K₂CO₃ (0.9 g, 6 mmol) and then the mixture was heated to 60° C. for 2 hours. After cooling to r.t., the mixture was filtered, and the filtrate concentrated and purified by chromatography on silica to give product 27c (0.99 g, 75.2%).

This compound was characterized by mass spectroscopy (MS) in accordance with the procedures described herein. Mass spectroscopy indicated MS (ESI): m/z 592 (M+H)⁺.

### Example 27

### Step D

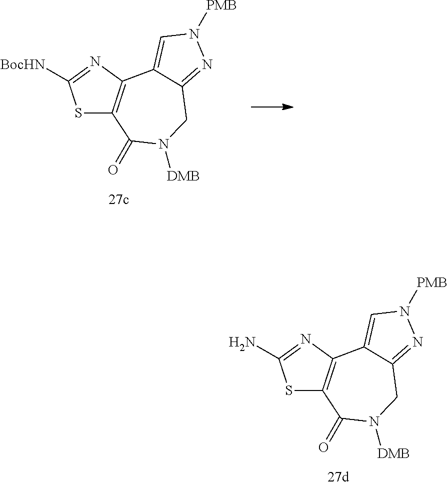

A solution of compound 27c (0.5 g, 0.85 mmol) in HCl (methanol solution, 10 mL) was stirred for 2 hours. The mixture was concentrated under reduced pressure to give the product 27d (0.395 mg, 95%).

This compound was characterized by mass spectroscopy (MS) in accordance with the procedures described herein. Mass spectroscopy indicated MS (ESI): m/z 492 (M+H)⁺.

### Example 27

### Step E

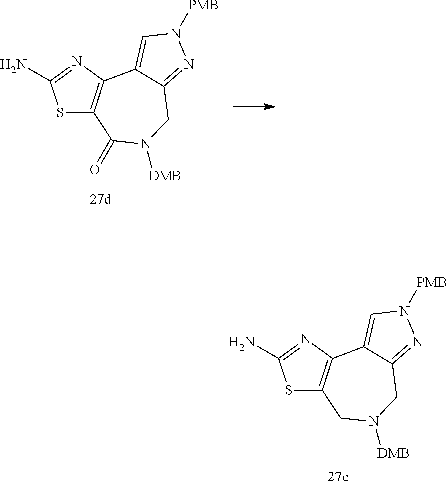

To a solution of compound 27d (395 mg, 0.80 mmol) in THF (2 mL) was added BH₃SMe₂ (2 M, 5 mL) at room temperature and then the mixture was heated to reflux for 2 hours. After cooling to r.t., the mixture was poured into water and extracted with EtOAc (3×25 mL). The combined organic layers were dried over MgSO₄, filtered and concentrated under reduced pressure. The residue was purified by chromatography on silica to afford compound 27e (130 mg, 34%).

This compound was characterized by mass spectroscopy (MS) in accordance with the procedures described herein. Mass spectroscopy indicated MS (ESI): m/z 478 (M+H)⁺.

### Example 27

### Step F

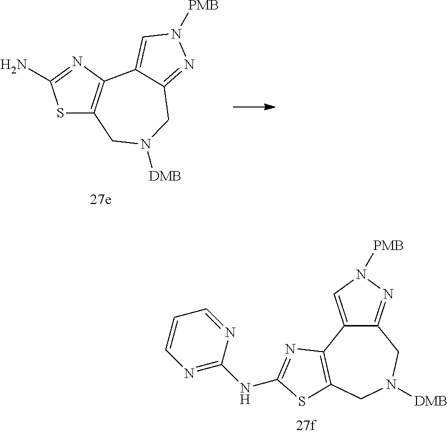

To a solution of compound 27e (300 mg, 0.63 mmol) and 2-chloropyrimidine (86 mg, 0.75 mmol) in dioxane (10 mL), under nitrogen, was added Pd₂(dba)₃ (30 mg), Xant-phos (38 mg) and Cs₂CO₃ (436 mg, 1.34 mmol). The reaction mixture was stirred for 2 hours at reflux, cooled to r.t., filtered and the residue washed with EtOAc (30 mL). The filtrate was concentrated under reduced pressure and the residue purified by preparative TLC to give product 27f (172 mg, 45%).

This compound was characterized mass spectroscopy (MS) in accordance with the procedures described herein. Mass spectroscopy indicated MS (ESI): m/z 556 (M+H)⁺.

### Example 27

### Step G

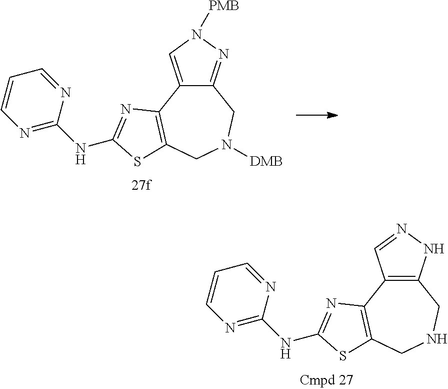

Compound 27f (172 mg, 0.3 mmol) in TFA (5 mL) and H₂O (0.5 mL) was heated to 100° C. in a microwave for 20 mins. The solution was cooled to r.t., then diluted with DMSO (5 mL) and filtered. The filtrate was collected and purified by preparative HPLC to give the product 27 as an off-white solid (71.3 mg, 83%).

This compound was characterized by proton NMR (¹HNMR) and mass spectroscopy (MS) in accordance with the procedures described herein. Proton NMR yielded the following results: ¹H NMR (DMSO-d6, 400 MHz): δ 11.86 (s, 1H), 9.74 (s, 2H), 8.63 (d, 2H, J=4.8 Hz), 7.94 (s, 1H), 7.05 (t, 1H, J=4.8 Hz), 4.65 (s, 2H), 4.61 (s, 2H). Mass spectroscopy indicated MS (ESI): m/z 286 (M+H)⁺.

### Example 28

Example 28 presents the preparation of Cmpd 28, 5-methyl-N-(4-methylpyrimidin-2-yl)-4,5,6,7-tetrahydropyrazolo[3,4-c][1,3]thiazolo[4,5-e]azepin-2-aminee (Table II, Compound No. 28).

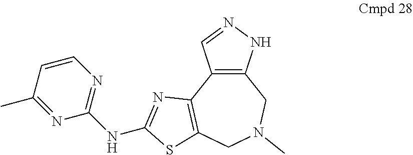

### Example 28

### Step A

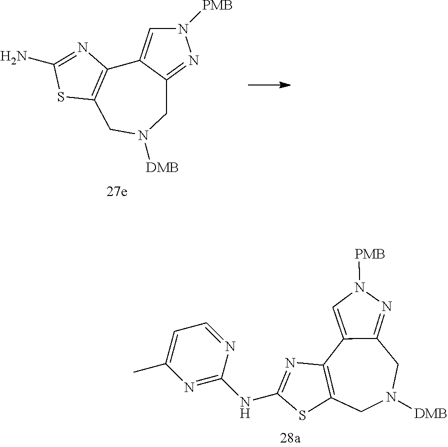

A mixture of compound 27e (300 mg, 0.63 mmol), 2-chloro-4-methylpyrimidine (86 mg, 0.75 mmol), Pd₂(dba)₃ (61 mg), Xant-phos (38 mg) and Cs₂CO₃ (436 mg, 1.34 mmol) in dioxane (5 mL) was heated to reflux for 2 hours under nitrogen. After cooling to r.t., the mixture was filtered and the residue was washed with EtOAc. The filtrate was concentrated under reduced pressure and purified by preparative TLC to give the product 28a (171.9 mg, 45%).

This compound was characterized by mass spectroscopy (MS) in accordance with the procedures described herein. Mass spectroscopy indicated MS (ESI): m/z 570 (M+H)⁺.

### Example 28

### Step B

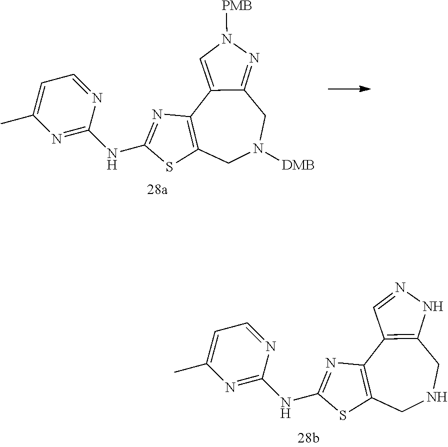

Compound 28a (172 mg, 0.3 mmol) was dissolved in 3 mL of TFA and the solution heated to 100° C. in a microwave for 20 mins. The solution was cooled to r.t. then diluted with DMSO (5 mL) and then filtered. The filtrate was collected and purified by preparative HPLC to give the product 28b as off-white solid (71 mg, 83%).

This compound was characterized by proton NMR (¹HNMR) and mass spectroscopy (MS) in accordance with the procedures described herein. Proton NMR yielded the following results: ¹H NMR (DMSO-d6, 400 MHz): δ 11.75 (s, 1H), 9.65 (s, 1H), 8.46 (d, 1H, J=5.2 Hz), 7.94 (s, 1H), 6.94 (d, 1H, J=5.2 Hz), 4.65 (s, 2H), 4.60 (s, 2H), 2.44 (s, 3H). Mass spectroscopy indicated MS (ESI): m/z 300 (M+H)⁺.

### Example 28

### Step C

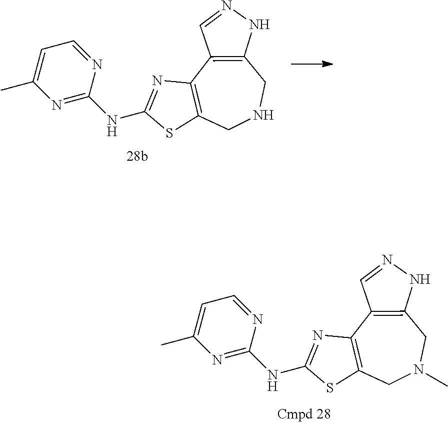

A solution of 28b (20 mg, 0.067 mmol), formaldehyde (50 mg), 4 A molecular sieves (200 mg) in MeOH (2 mL) was treated with AcOH (0.05 mL) and the mixture was stirred for 30 mins. Sodium cyanoborohydride (8.4 mg, 0.134 mmol) was added and the mixture was stirred for 18 h at r.t. The mixture was filtered and concentrated under reduced pressure and the residue purified by preparative HPLC to give the product 28 as white solid (2.1 mg, 10%).

This compound was characterized by proton NMR (¹HNMR) and mass spectroscopy (MS) in accordance with the procedures described herein. Proton NMR yielded the following results: ¹H NMR (DMSO-d6, 400 MHz): δ 11.81 (s, 1H), 8.47 (d, 1H, J=4.8 Hz), 7.97 (s, 1H), 6.95 (d, 1H, J=4.8 Hz), 4.79 (s, 4H), 2.98 (s, 3H), 2.31 (s, 3H). Mass spectroscopy indicated MS (ESI): m/z 314 (M+H)⁺.

### Example 29

Example 29 presents the preparation of Cmpd 29, N-(5-fluoropyrimidin-2-yl)-4,5,6,7-tetrahydropyrazolo[3′,4′:6,7]cyclohepta[1,2-d][1,3]thiazol-2-amine (Table II, Compound No. 29)

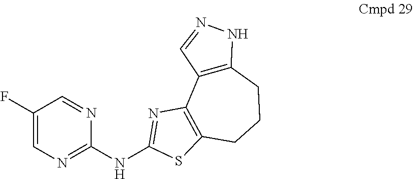 [SMILES: Fc1cnc(Nc2nc3c(s2)CCCc2[nH]ncc2-3)nc1]

### Example 29

### Step A

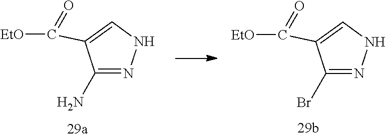 [SMILES: CCOC(=O)c1c[nH]nc1N ; CCOC(=O)c1c[nH]nc1Br]

To a solution of t-BuONO (124 g, 1.20 mol) in 3 L MeCN was added CuBr₂ (279 g, 1.20 mol). The mixture was stirred at room temperature for 1 h under nitrogen, then compound 29a (160 g, 1.03 mol) was added in portions over 30 min. The mixture was stirred at room temperature under nitrogen for 30 min, then heated to 70° C. and stirred for an additional 2 h. After cooling to r.t., the solvent was removed in vacuo. The residue was diluted with EtOAc (3 L) and washed with brine (400 mL×3). The organic layer was dried over MgSO₄, filtered, and concentrated to afford the crude product 29b (200 g, 89% yield) used directly in the next step without purification.

### Example 29

### Step B

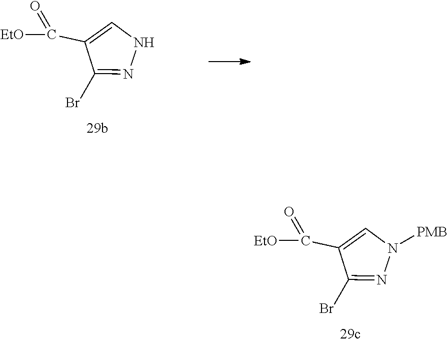

A mixture of compound 29b (200 g, 0.91 mol), PMBCl (213 g, 1.36 mol) and K₂CO₃ (188 g, 136 mol) in 2 L of MeCN was heated to reflux for 18 h. After cooling to r.t., the reaction mixture was filtered and the solid washed with DCM (400 mL×5). The filtrate was collected, dried over Na₂SO₄, filtered and concentrated under reduced pressure. The residue was purified by chromatography on silica (PE:EtOAc=30:1˜10:1) to afford compound 29c as an off-white solid (120 g, 39%).

This compound was characterized by proton NMR (¹HNMR) and mass spectroscopy (MS) in accordance with the procedures described herein. Proton NMR yielded the following results: ¹H NMR (CDCl₃, 400 MHz): δ 7.70 (s, 1H), 7.21 (d, 2H, J=8.4 Hz), 6.88 (d, 2H, J=8.4 Hz), 5.17 (s, 2H), 4.25 (q, 2H, J=7.2 Hz), 3.79 (s, 3H), 1.30 (t, 3H, J=7.2 Hz. Mass spectroscopy indicated MS (ESI): m/z 339, 341 (M+H)⁺.

### Example 29

### Step C

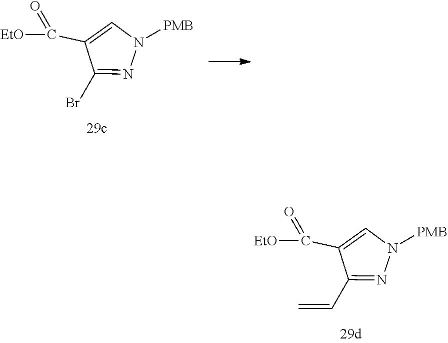

A mixture of compound 29c (100 g, 295 mmol), tributyl(vinyl)tin (112 g, 354 mmol) and Pd(PPh₃)₄ (17 g, 15 mmol) in 500 mL of toluene was heated to reflux for 6 h. After cooling to r.t., the mixture was concentrated under reduced pressure and the residue purified by chromatography on silica column (PE:EtOAc=10:1) give compound 29d (52 g, 62%).

This compound was characterized by mass spectroscopy (MS) in accordance with the procedures described herein. Mass spectroscopy indicated MS (ESI): m/z 287 (M+H)⁺.

### Example 29

### Step D

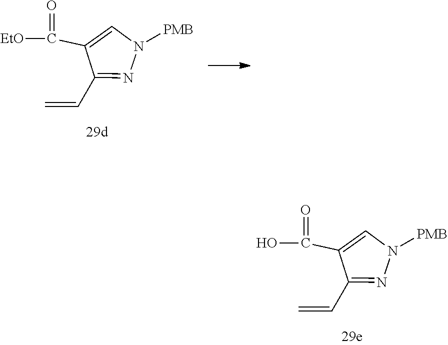

To a solution of compound 29d (52 g, 181 mmol) in CH₃OH (200 mL) was added 4N NaOH (100 mL). The reaction mixture was heated to reflux for 2 h., cooled to r.t. and the pH adjusted to 2-3 with conc. HCl and the reaction mixture extracted with EtOAc (250 mL×5). The combined organic layers were washed with water, dried over MgSO₄, filtered, and concentrated in vacuo to afford the product 29e (43 g, 92% yield), used without purification.

### Example 29

### Step E

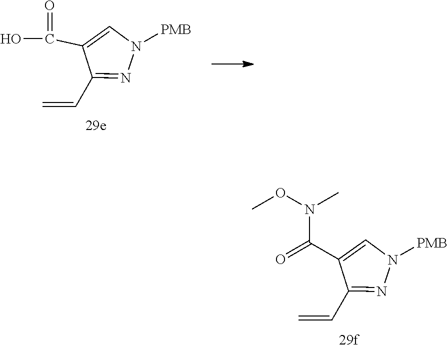

To a solution of 29e (43 g, 166 mmol) and five drops of DMF in DCM (300 mL) was added oxalyl chloride (42 g, 332 mmol) dropwise at 0° C. under N₂. The reaction mixture was allowed to warm up to r.t. and stirred for 3 h. After evaporation of the solvent, the residue was treated with toluene and coevaporated to dryness to yield the acid chloride.

To a mixture of N,O-dimethylhydroxylamine hydrochloride (24 g, 249 mmol) and TEA (67 g, 664 mmol) in DCM (250 mL) was added dropwise a solution of the acid chloride in DCM (70 mL) at 0° C. under N₂. The reaction mixture was allowed to warm up to room temperature and stirred overnight. The reaction mixture was subsequently washed with brine (100 mL×3) and then water (100 mL×2). It was then dried over MgSO₄, filtered, and concentrated. The residue was purified by chromatography on silica (PE:EtOAc=10:1˜4:1) to afford compound 29f as off-white solid (32 g, 64%).

This compound was characterized by mass spectroscopy (MS) in accordance with the procedures described herein. Mass spectroscopy indicated MS (ESI): m/z 302 (M+H)⁺.

### Example 29

### Step F

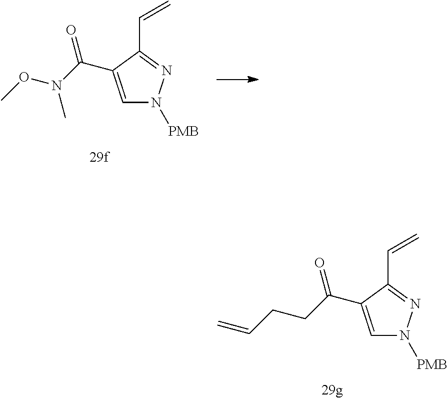

Magnesium (8.6 g, 0.36 mol) and dry THF (100 mL) were introduced under inert atmosphere into a three-necked flask. A solution of 4-bromo-1-butene (27 g, 0.2 mol) in dry THF (300 mL) was introduced into the dropping funnel. About 30 mL of this solution was added first to initiate the reaction. The remaining solution was added dropwise while maintaining the temperature between 60-70° C.

To a solution of 29f (30 g, 0.1 mol) in dry THF (200 mL) was added dropwise the above Grignard reagent at −78° C. under N₂. The reaction mixture was stirred at −78° C. for 30 min, and then allowed to warm to r.t. overnight. Saturated NH₄Cl (100 mL) was added and most of the THF removed under vacuum. The resulting mixture was extracted with DCM (150 mL×4) and the combined organic phases dried over Na₂SO₄, filtered and concentrated to give the crude product which was purified by chromatography on silica (PE:EtOAc=10:1) to afford product 29g (15 g, 51% yield).

This compound was characterized by mass spectroscopy (MS) in accordance with the procedures described herein. Mass spectroscopy indicated MS (ESI): m/z 297 (M+H)⁺.

### Example 29

### Step G

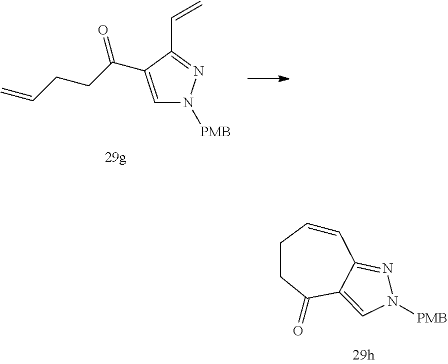

To a solution of 29g (4 g, 13.5 mmol) in dry DCM (1800 mL) was added Grubbs Catalyst 2^{nd} Generation [1,3-Bis(2,4,6-trimethylphenyl)-2-(imidazolidinylidene)(dichlorophenylmethylene)(tricyclohexylphosphine)ruthenium, CAS 246047-72-3] (0.573 g, 0.67 mmol) under N₂, then the reaction mixture was refluxed for 18 h. After cooling to r.t., the solvent was removed under reduced pressure and the residue was purified by flash column chromatography (PE:EtOAc=10:1-5:1) to afford the desired compound 29h (3 g, 83% yield).

This compound was characterized by mass spectroscopy (MS) in accordance with the procedures described herein. Mass spectroscopy indicated MS (ESI): m/z 269 (M+H)⁺.

### Example 29

### Step H

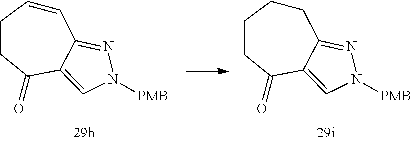

To a solution of compound 29h (400 mg, 1.5 mmol) in MeOH (30 mL) was added Pd/C (50 mg). The reaction mixture was stirred at room temperature for 2 h under 1 atm hydrogen. The mixture was filtered and the filtrate concentrated under reduced pressure to afford the product 29i (400 mg, 100%), used directly without further purification.

This compound was characterized by mass spectroscopy (MS) in accordance with the procedures described herein. Mass spectroscopy indicated MS (ESI): m/z 271 (M+H)⁺.

### Example 29

### Step 1

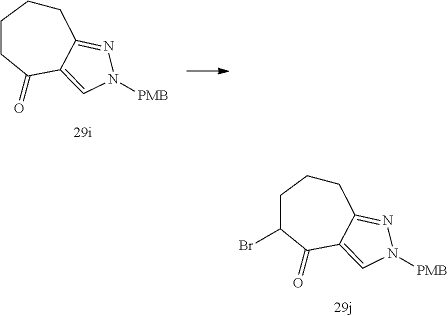

A mixture of compound 29i (270 mg, 1.0 mmol) and PhNMe₃Br₃ (376 mg, 1.0 mmol) in CHCl₃ (6 mL) was heated to reflux for 30 min. After cooling to r.t., the mixture was diluted with DCM (40 mL), washed with brine (10 mL×2) and water (10 mL), dried over Na₂SO₄, filtered and concentrated to afford the crude product 29j (320 mg, 92%), used without further purification.

### Example 29

### Step J

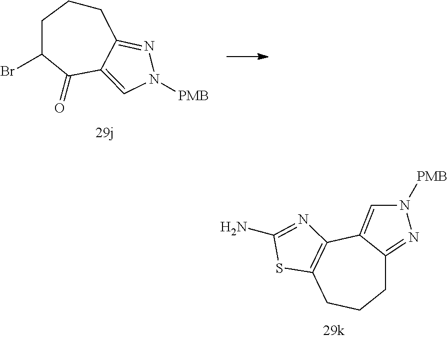

A mixture of compound 29j (320 mg, 0.9 mmol) and thiourea (76 mg, 1.0 mmol) in EtOH (8 mL) was heated to reflux for 2 hours. After cooling to r.t., the mixture was concentrated in vacuo. The residue was purified by preparative TLC (DCM: CH₃OH=20:1) to afford the desired compound 29k (232 mg, 79% yield).

This compound was characterized by mass spectroscopy (MS) in accordance with the procedures described herein. Mass spectroscopy indicated MS (ESI): m/z 327 (M+H)⁺.

### Example 29

### Step K

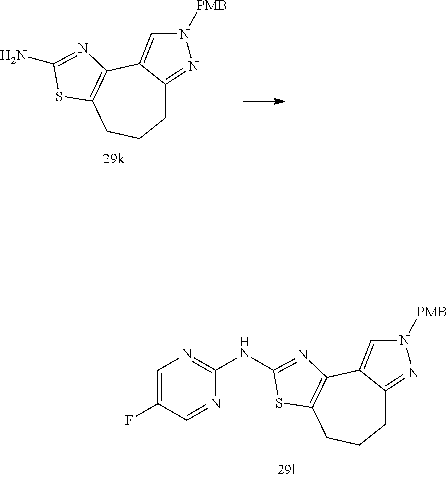

A mixture of 29k (232 mg, 0.71 mmol), 2-chloro-5-fluoropyrimidine (111 mg, 0.84 mmol), Pd2(dba)₃ (64 mg, 0.07 mmol), Xant-phos (81 mg, 0.14 mmol) and Cs₂CO₃ (456 mg, 1.4 mmol) in dioxane (5 mL) was heated to reflux for 4 h under N₂. After cooling to r.t., the reaction mixture was filtered and concentrated under reduced pressure. The residue was purified by preparative TLC (DCM: CH₃OH=10:1) to afford the desired compound 29l (126 mg, 42% yield).

This compound was characterized by mass spectroscopy (MS) in accordance with the procedures described herein. Mass spectroscopy indicated MS (ESI): m/z 423 (M+H)⁺.

### Example 29

### Step L

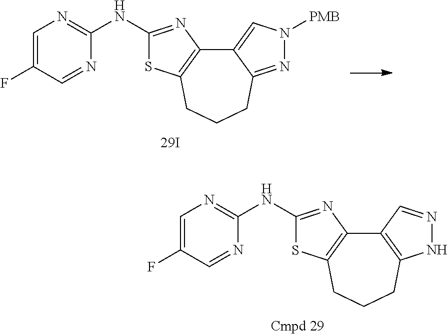

A solution of compound 291 (126 mg, 0.3 mmol) in TFA (4 mL) was heated at 100° C. in a microwave for 20 min. After cooling to r.t., the mixture was diluted with DCM and concentrated in vacuo. The residue was purified by preparative HPLC to give the desired product Example 29 (20 mg, 22%).

This compound was characterized by proton NMR (¹HNMR) and mass spectroscopy (MS) in accordance with the procedures described herein. Proton NMR yielded the following results: ¹H NMR (DMSO-d6, 400 MHz): 611.653 (br, 1H,), 8.678 (s, 2H), 7.688 (s, 1H), 2.939-2.964 (m, 4H), 1.964 (s, 2H). Mass spectroscopy indicated MS (ESI): m/z 303 (M+H)⁺.

### Example 34

Example 34 presents the preparation of Cmpd 34, 6,6-difluoro-N-(4-methylpyrimidin-2-yl)-4,5,6,7-tetrahydropyrazolo[3′,4′:6,7]cyclohepta[1,2-d][1,3]thiazol-2-amine (Table II, Compound No. 34)

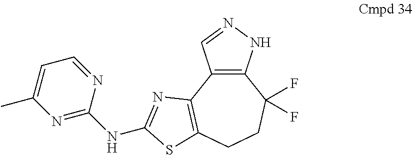 [SMILES: Cc1ccnc(Nc2nc3c(s2)CCC(F)(F)c2[nH]ncc2-3)n1]

### Example 34

### Step A

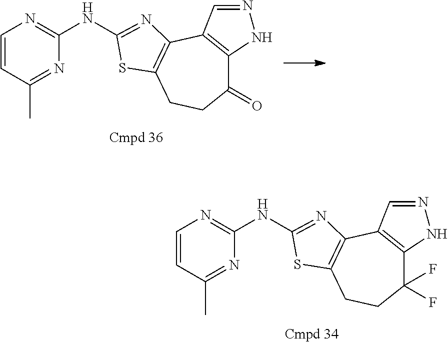 [SMILES: Cc1ccnc(Nc2nc3c(s2)CCC(=O)c2[nH]ncc2-3)n1 ; Cc1ccnc(Nc2nc3c(s2)CCC(F)(F)c2[nH]ncc2-3)n1]

To a solution of Cmpd 36 (10 mg, 0.032 mmol, preparation described in Example 36) in 2 mL of DCM, DAST (51.5 mg, 0.32 mmol) was added dropwise at 0° C. The resulting mixture was stirred at 40° C. for 3 h. After 3 hours the mixture was cooled to r.t. and concentrated in vacuo. The residue was diluted with water, then treated with K₂CO₃ to pH=8, then extracted with EtOAc (10 mL×2). The combined organic layers were dried over Na₂SO₄, filtered and concentrated in vacuo. The residue was purified by preparative HPLC to yield Example 34 (3.5 mg, 32.7%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.41 (d, J=4.4 Hz, 1H), 7.89 (s, 1H), 6.88 (d, J=4.4 Hz, 1H), 2.99 (t, J=4.4 Hz, 2H), 2.50-2.51 (m, 2H), 2.40 (s, 3H). Mass spectroscopy indicated MS (ESI): m/z 335 (M+H)⁺.

### Example 36

Example 36 presents the preparation of Cmpd 36, 2-[(4-methylpyrimidin-2-yl)amino]-4,7-dihydropyrazolo[3′,4′:6,7]cyclohepta[1,2-d][1,3]thiazol-6(5H)-one (Table II, Compound No. 36)

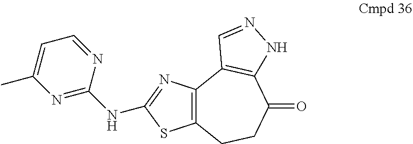 [SMILES: Cc1ccnc(Nc2nc3c(s2)CCC(=O)c2[nH]ncc2-3)n1]

### Example 36

### Step A

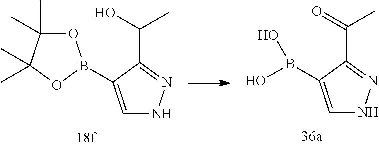 [SMILES: CC(O)c1n[nH]cc1B1OC(C)(C)C(C)(C)O1 ; CC(=O)c1n[nH]cc1B(O)O]

To a solution of 18f (9 g, 25 mmol) in 100 mL of DCM, with stirring, was added PCC (10.7 g, 50 mmol) at rt. After two hours of additional stirring at r.t. the mixture was concentrated in vacuo to give the product 36a (4.6 g, 68.0%) which was used for next step without further purification.

This compound was characterized by mass spectroscopy (MS) in accordance with the procedures described herein. Mass spectroscopy indicated MS (ESI): m/z 275 (M+H)⁺.

### Example 36

### Step B

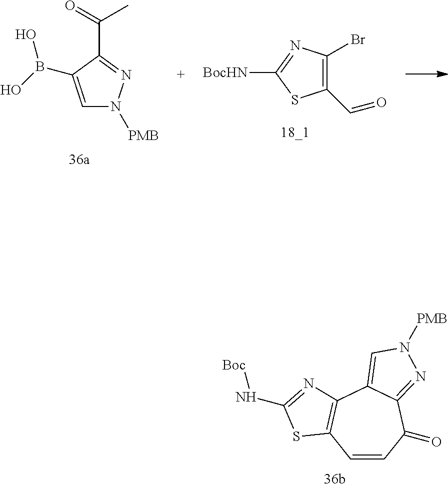

A mixture of 36a (2.0 g, 6.5 mmol), 18_{—}1 (2.14 g, 7.8 mmol), Pd(PPh₃)₂Cl₂ (200 mg) and K₃PO₄ (5.2 g, 19.5 mmol) in a mixed solvent of DMF (70 mL) and water (15 mL) was heated to 100° C. for 2 h under nitrogen. Subsequently, the reaction was cooled to r.t. and then water (40 mL) was added. The mixture was extracted with EtOAc (80 mL×3). The combined organic layers were dried over Na₂SO₄, filtered and concentrated under reduced pressure. The residue was purified by silica chromatography (PE: EA=3:1 to 1:2) to give product 36b (1.6 g, 56.1%). This product compound was characterized by mass spectroscopy (MS) in accordance with the procedures described herein. Mass spectroscopy indicated MS (ESI): m/z 439 (M+H)⁺.

### Example 36

### Step C

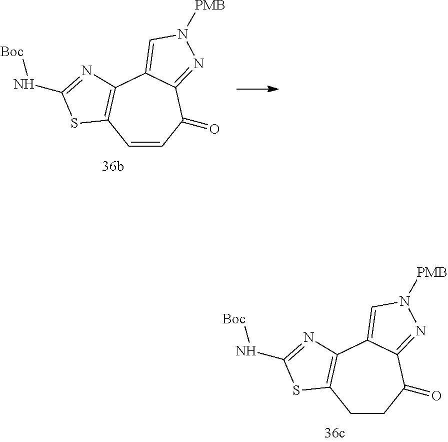

To a solution of compound 36b (700 mg, 1.6 mol) in EtOAc (20 mL) was added PtO₂ (100 mg). The resulting mixture was stirred at 50° C. under H₂ (50 PSi.) for 15 hrs. Subsequently the mixture was cooled to r.t. and then filtered through Celite. The filtrate was concentrated in vacuo and the residue was purified by preparative TLC (PE: EtOAc=1:1) to give compound 36c (200 mg, 28.0%).

This compound was characterized by proton NMR (¹HNMR) and mass spectroscopy (MS) in accordance with the procedures described herein. Proton NMR yielded the following results: ¹H NMR (CDCl₃, 400 MHz): δ 7.67 (s, 1H), 7.29 (d, J=8.4 Hz, 2H), 6.91 (d, J=8.4 Hz, 2H), 5.35 (s, 2H), 3.82 (s, 3H), 3.02 (s, 4H), 1.53 (s, 9H). Mass spectroscopy indicated MS (ESI): m/z 441 (M+H)⁺.

### Example 36

### Step D

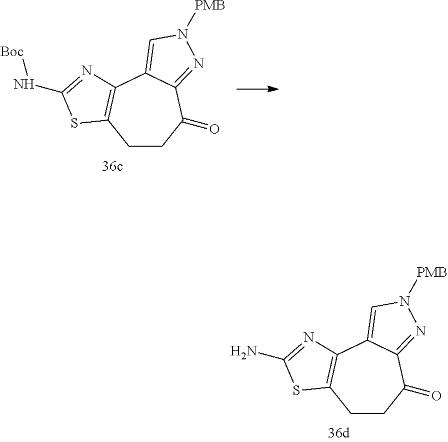

A solution of compound 36c (150 mg, 0.35 mmol) in TFA (1 mL) and DCM (4 mL) was stirred at r.t. for 4 h. The mixture was concentrated in vacuo, diluted with water, then treated with aqueous K₂CO₃ to pH=8, and then extracted with EtOAc (10 mL×2). The combined organic layers were dried over Na₂SO₄, then filtered and concentrated in vacuo to give compound 36d (100 mg, 86.3%).

This compound was characterized by mass spectroscopy (MS) in accordance with the procedures described herein. Mass spectroscopy indicated MS (ESI): m/z 341 (M+H)⁺.

### Example 36

### Step E

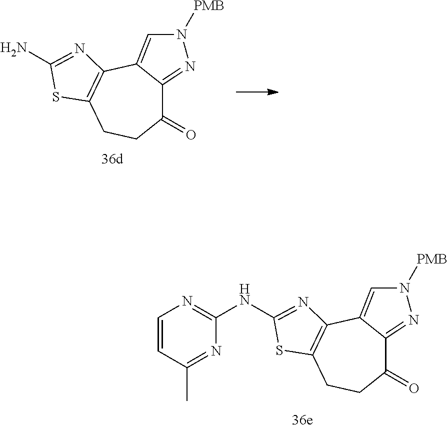

A mixture of compound 36d (100 mg, 0.29 mmol), 2-chloro-4-methylpyrimidine (75 mg, 0.58 mmol), Pd₂(dba)₃ (26 mg, 0.029 mmol), Xant-phos (34 mg, 0.058 mmol) and Cs₂CO₃ (189 mg, 0.58 mmol) in dioxane (4 mL) was heated to reflux under N₂. Reflux was maintained for 3 hrs under N₂ then cooled to r.t. Subsequently the mixture was filtered and concentrated in vacuo to give product 36e (55 mg, 43.9%).

This compound was characterized by mass spectroscopy (MS) in accordance with the procedures described herein. Mass spectroscopy indicated MS (ESI): m/z 433 (M+H)⁺.

### Example 36

### Step F

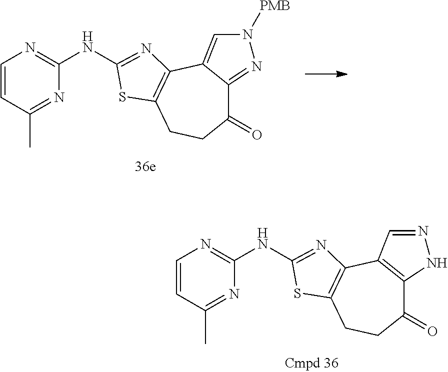

Compound 36e (55 mg, 0.127 mmol) in TFA (2 mL) was stirred at 100° C. for 1 h. Subsequently the mixture was cooled to r.t. and concentrated in vacuo. The residue was purified by preparative HPLC to yield the desired product, Cmpd 36 (25 mg, 62.9%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.42 (d, J=4.8 Hz, 1H), 7.82 (s, 1H), 6.88 (d, J=4.8 Hz, 1H), 2.99 (t, J=4.8 Hz, 2H), 2.82 (t, J=4.8 Hz, 2H), 2.41 (s, 3H). Mass spectroscopy indicated MS (ESI): m/z 313 (M+H)⁺.

### Example 37

Example 37 presents the preparation of Cmpd 37, 5,5-dimethyl-2-[(4-methylpyrimidin-2-yl)amino]-4,7-dihydropyrazolo[3′,4′:6,7]cyclohepta[1,2-d][1,3]thiazol-6(5H)-one (Table II, Compound No. 37).

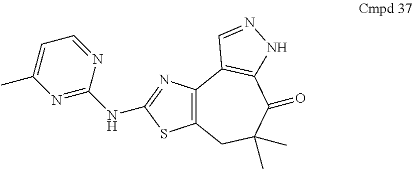 [SMILES: Cc1ccnc(Nc2nc3c(s2)CC(C)(C)C(=O)c2[nH]ncc2-3)n1]

### Example 37

### Step A

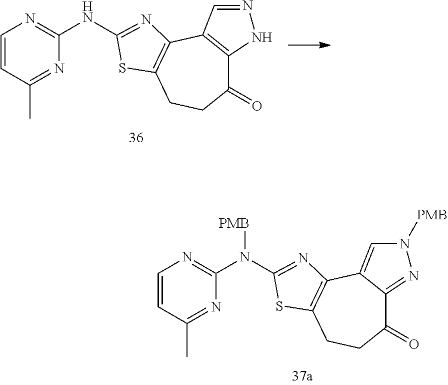

To a suspension of Example 36 (10 mg, 0.032 mmol) and K₂CO₃ (16 mg, 0.112 mmol) in CH₃CN (2 mL) was added PMBCl (10.4 mg, 0.067 mmol). The resulting mixture was heated to reflux and reflux was maintained for 1 hr. Subsequently the mixture was cooled to r.t., then diluted with H₂O (5 mL) and extracted with EtOAc (5 mL×3). The combined organic layers were dried over Na₂SO₄, filtered and the filtrate was concentrated in vacuo to give crude product 37a (15 mg, 84.7%). The product thus obtained was used directly in the subsequent step.

This compound was characterized by mass spectroscopy (MS) in accordance with the procedures described herein. Mass spectroscopy indicated MS (ESI): m/z 553 (M+H)⁺.

### Example 37

### Step B

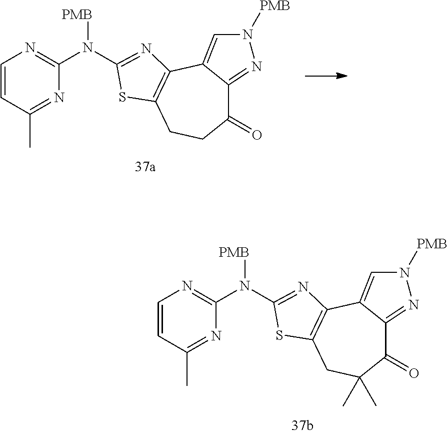

To a solution of 37a (15 mg, 0.027 mmol) and iodomethane (8.4 mg, 0.059 mmol) in THF (2 mL) was added NaH (3.3 mg, 0.081 mmol, 60% in oil) at 0° C. The resulting mixture was allowed to warm up to r.t. and stirred for 40 min. Subsequently, water (5 mL) was added and the mixture extracted with EtOAc (5 mL×3). The combined organic layers were dried over Na₂SO₄, filtered and concentrated under reduced pressure to give product 37b (13 mg, 82.8%).

This compound was characterized by mass spectroscopy (MS) in accordance with the procedures described herein. Mass spectroscopy indicated MS (ESI): m/z 581 (M+H)⁺.

### Example 37

### Step C

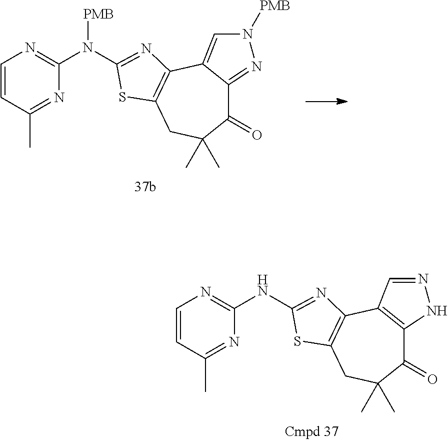

Compound 37b (13 mg, 0.022 mmol) in TFA (2 mL) was stirred at 100° C. for 1 h. Subsequently the mixture was cooled to r.t. and concentrated in vacuo. The residue was purified by preparative HPLC to yield product Example 37 (3 mg, 40.0%).

This compound was characterized by proton NMR (¹HNMR) and mass spectroscopy (MS) in accordance with the procedures described herein. Proton NMR yielded the following results: ¹H NMR (DMSO-d6, 400 MHz): δ 8.43 (d, J=4.8 Hz, 1H), 7.81 (s, 1H), 6.88 (d, J=4.8 Hz, 1H), 2.94 (s, 2H), 2.41 (s, 3H), 1.09 (s, 6H). Mass spectroscopy indicated MS (ESI): m/z 341 (M+H)⁺.

### Example 38

Example 38 presents the preparation of Cmpd 38 (Table II, Compound No. 38)

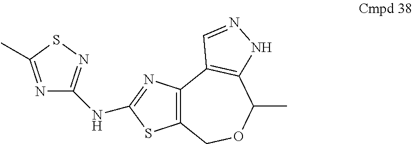

### Example 38

### Step A

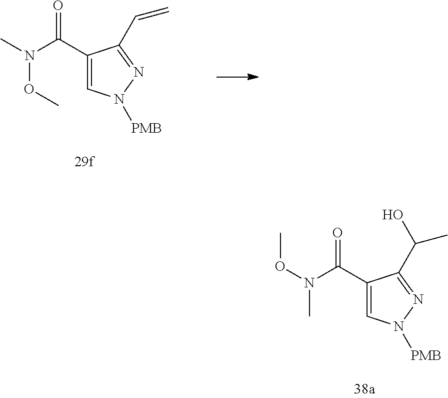

A mixture of 29f (20 g, 66 mmol) and Hg(OAc)₂ (36 g, 112 mmol.) in THF (70 mL) and H₂O (105 mL) was stirred for 15 h at r.t., and then NaOH (5 g, 125 mmol) was added. After 10 min, NaBH₄ (4 g, 105 mmol) was added in portions at −20° C. The mixture was allowed to warm up to r.t. and stirred for 1 h, then diluted with water, filtered and extracted with EtOAc (3×100 mL). The combined organic layers were dried over Na₂SO₄, filtered and concentrated under reduced pressure. The residue was purified by silica gel column (PE: EA=2:1) to give compound 38a (7.4 g, 35%).

This compound was characterized by proton NMR (¹HNMR) and mass spectroscopy (MS) in accordance with the procedures described herein. Proton NMR yielded the following results: ¹H NMR (CDCl₃, 400 MHz): δ 7.80 (s, 1H), 7.17 (d, J=8.4 Hz, 2H), 6.86 (d, J=8.4 Hz, 2H), 5.18 (s, 2H), 4.98 (q, J=6.4 Hz, 1H), 3.78 (s, 3H), 3.56 (s, 3H), 3.29 (s, 3H), 1.57 (d, J=6.4 Hz, 3H). Mass spectroscopy indicated MS (ESI): m/z 320 (M+H)⁺.

### Example 38

### Step B

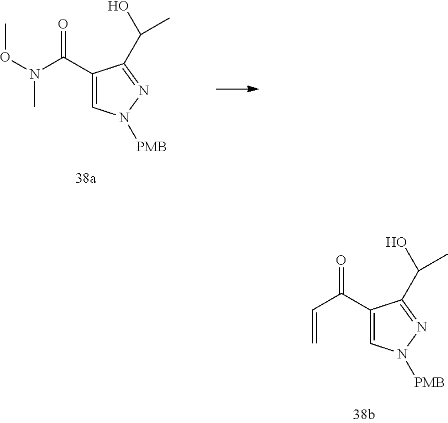

To a solution of compound 38a (5 g, 15.7 mmol) in THF (50 mL) was added vinyl magnesium bromide (1M, 47 mL, 47 mmol) dropwise at −20° C. The mixture was allowed to warm up to r.t. and stirred for 30 min, then quenched with cooled saturated NH₄Cl (aq) and extracted with EtOAc (30 mL×2). The combined organic layers were dried over Na₂SO₄, filtered and concentrated under reduced pressure. The residue was purified by silica gel column (PE: EA=2:1) to give compound 38b (1.8 g, 40%).

This compound was characterized by proton NMR (¹HNMR) and mass spectroscopy (MS) in accordance with the procedures described herein. Proton NMR yielded the following results: ¹H NMR (CDCl₃, 400 MHz): δ 7.69 (s, 1H), 7.17 (d, J=8.8 Hz, 2H), 6.85 (d, J=8.8 Hz, 2H), 6.72 (dd, J=10.4 Hz, 17.6 Hz, 2H), 6.37 (d, J=17.6 Hz, 2H), 5.73 (d, J=10.4 Hz, 2H), 5.36 (d, J=8.0 Hz, 2H), 5.15 (s, 2H), 4.90-4.97 (m, 1H), 3.76 (s, 3H), 1.50 (d, J=6.0 Hz, 3H) Mass spectroscopy indicated MS (ESI): m/z 287 (M+H)⁺.

### Example 38

### Step C

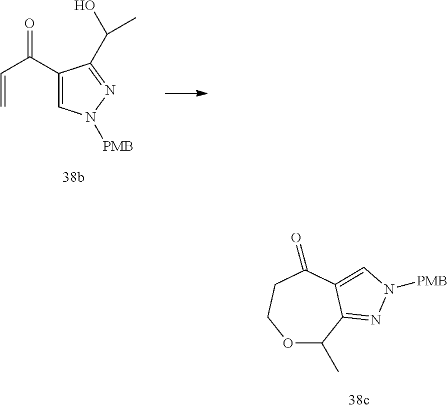

To a solution of compound 38b (1.8 g, 6.3 mmol) in DCM (20 mL) was added BF₃.Et₂O (0.9 g, 6.3 mmol) dropwise at −20° C. The mixture was allowed to warm up to r.t. and stirred for 15 h. Subsequently the mixture was quenched with saturated Na₂CO₃ (aq) and extracted with EtOAc (30 mL×2). The combined organic layers were dried over Na₂SO₄, filtered and concentrated under reduced pressure. The residue was purified by silica gel column (PE: EA=2:1) to give compound 38c (0.44 g, 24.4%).

This compound was characterized by proton NMR (¹HNMR) and mass spectroscopy (MS) in accordance with the procedures described herein. Proton NMR yielded the following results: ¹H NMR (CDCl₃, 400 MHz): δ 7.72 (s, 1H), 7.15 (d, J=8.4 Hz, 2H), 6.83 (d, J=8.4 Hz, 2H), 5.09 (s, 2H), 4.85 (q, J=6.4 Hz, 1H), 4.12-4.15 (m, 1H), 3.79-3.85 (m, 1H), 3.75 (s, 3H), 2.88-2.96 (m, 1H), 2.57-2.62 (m, 1H), 1.57 (d, J=6.4 Hz, 3H). Mass spectroscopy indicated MS (ESI): m/z 287 (M+H)⁺.

### Example 38

### Step D

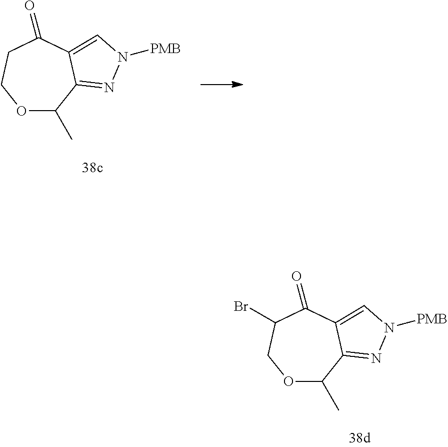

A mixture of 38c (220 mg, 0.77 mmol) and trimethylphenylammomium tribromide (200 mg, 0.53 mmol.) in DCM (10 mL) was heated to reflux. Reflux was maintained for one hour, then the mixture was cooled, washed with water (3 mL), dried over Na₂SO₄, filtered and concentrated under reduced pressure to give crude product 5 (260 mg, 92.8%) which was used for next step without further purification.

This compound was characterized by mass spectroscopy (MS) in accordance with the procedures described herein. Mass spectroscopy indicated MS (ESI): m/z 365, 367 (M+H)⁺.

### Example 38

### Step E

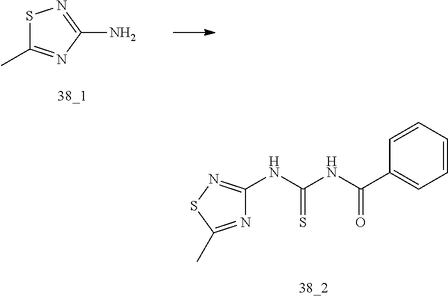

To NH₄SCN (0.55 g, 7.3 mmol) in acetone (10 mL) was added benzoyl chloride (0.9 g, 6.5 mmol) dropwise at −10° C. The mixture was stirred for 2 h at 0° C., then filtered. Compound 38_{—}1 (0.6 g, 5.2 mmol) was added to the filtrate. The mixture was stirred for 15 h at 20° C. and then concentrated under reduced pressure to give compound 38_{—}2 (1.44 g, 100%) which was used in the next step without further purification.

This compound was characterized by mass spectroscopy (MS) in accordance with the procedures described herein. Mass spectroscopy indicated MS (ESI): m/z 279 (M+H)⁺.

### Example 38

### Step F

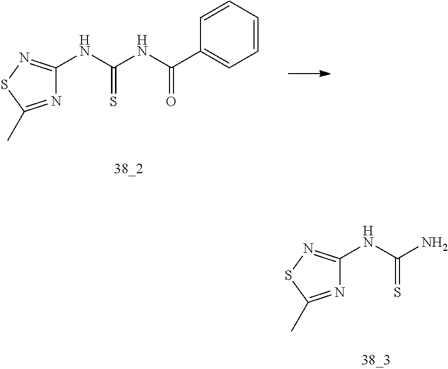

A solution of compound 38_{—}2 (1.4 g, 5.2 mmol) in NaOH (10 mL, 3N) was heated for 3 h at 80° C., then cooled to r.t. and concentrated under reduced pressure. The residue was purified by preparative HPLC to give compound 38_{—}3 (0.3 g, 33%).

This compound was characterized by mass spectroscopy (MS) in accordance with the procedures described herein. Mass spectroscopy indicated MS (ESI): m/z 175 (M+H)⁺.

### Example 38

### Step G

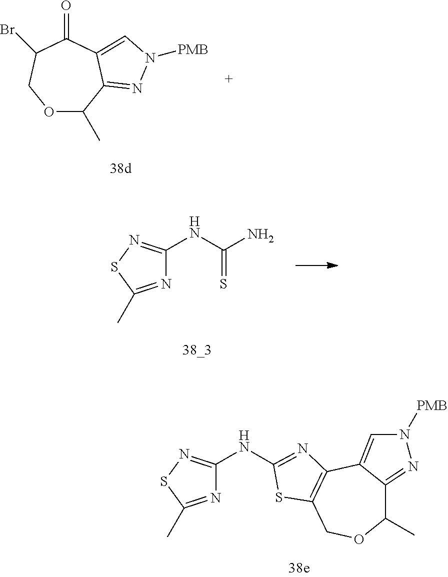

A mixture of compound 38 (100 mg, 0.27 mmol) and 38_{—}3 (150 mg, 0.8 mmol) in EtOH (4 mL, 10%) was heated to reflux. Reflux was maintained for 3 hours, and then the mixture was cooled to r.t. and concentrated under reduced pressure. The residue was purified by crystallizing from DCM:MeOH (3:1) to give compound 38e (90 mg, 76%).

This compound was characterized by mass spectroscopy (MS) in accordance with the procedures described herein. Mass spectroscopy indicated MS (ESI): m/z 441 (M+H)⁺.

### Example 38

### Step H

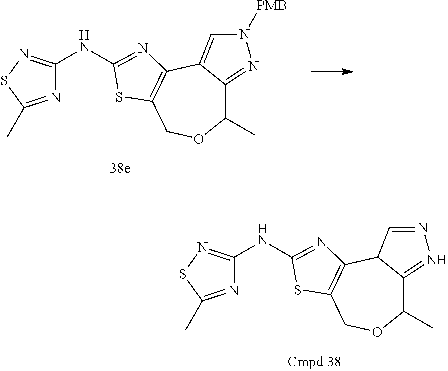

Compound 38e (90 mg, 0.2 mmol) in TFA (3 mL) and H₂O (0.1 mL) was heated to reflux. Reflux was maintained for one hour. After one hour at reflux the mixture was cooled to rt. and concentrated under reduced pressure. The residue was purified by preparative HPLC to give the desired compound, Cmpd 38 (30 mg, 49%).

This compound was characterized by proton NMR (¹HNMR) and mass spectroscopy (MS) in accordance with the procedures described herein. Proton NMR yielded the following results: ¹H NMR (DMSO-d6, 400 MHz): δ 12.12 (br, 1H), 7.74 (s, 1H), 4.84-5.04 (m, 3H), 2.72 (s, 3H), 1.51 (d, J=6.4 Hz, 3H). Mass spectroscopy indicated MS (ESI): m/z 321 (M+H)⁺.

### Example 40

Example 24 presents the preparation of 4,6-Dimethyl-N-(4-methylpyrimidin-2-yl)-6,7-dihydro-4H-pyrazolo[4′,3′:5,6]oxepino-[4,3-d][1,3]thiazol-2-amine (Table II, Compound No. 24)

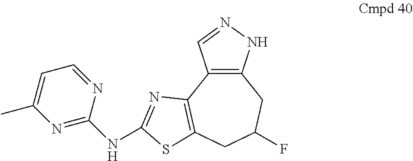 [SMILES: Cc1ccnc(Nc2nc3c(s2)CC(F)Cc2[nH]ncc2-3)n1]

### Example 40

### Step A

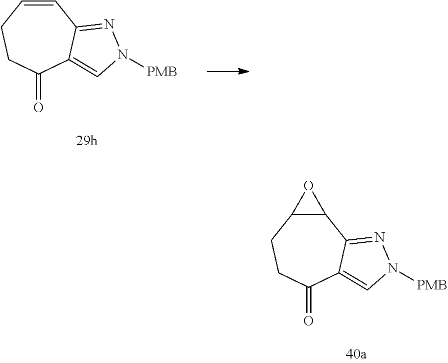

To a solution of 29h (5.36 g, 20 mmol) in dry DCM (100 mL) was added m-CPBA (6.90 g, 40 mmol) in portions at 0° C. The resulting mixture was allowed to warm up to room temperature and stirred for 5 h. Subsequently, the mixture was diluted with DCM (200 mL), and then washed in sequence with aqueous Na₂SO₃ (50 mL×3), aqueous NaHCO₃ (30 mL×3) and H₂O (30 mL×2). The organic layer was dried over Na₂SO₄, filtered and concentrated to afford the product (4.82 g, 85%), used subsequently without purification.

This compound was characterized by mass spectroscopy (MS) in accordance with the procedures described herein. Mass spectroscopy indicated MS (ESI): m/e 285 (M+H)⁺.

### Example 40

### Step B

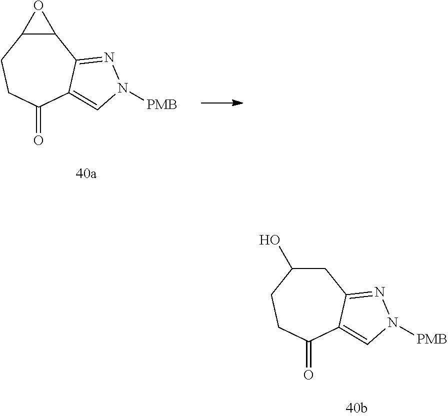

To a solution of compound 40a (4.82 g, 17 mmol) in MeOH (80 mL) was added Pd/C (0.8 g). The resulting mixture was stirred at room temperature for 12 h under hydrogen atmosphere. The mixture was filtered, concentrated under reduced pressure, then purified by flash column chromatography (PE:EA=5:1-1:1) to afford the desired compound 40b (1.72 g, 35% yield).

This compound was characterized by proton NMR (¹HNMR) and mass spectroscopy (MS) in accordance with the procedures described herein. Proton NMR yielded the following results: ¹H NMR (CDCl₃, 400 MHz): δ 7.70 (s, 1H,), 7.15 (d, 2H, J=8.4 Hz), 6.82 (d, 2H, J=8.4 Hz), 5.08 (s, 2H), 4.24-4.30 (m, 1H), 3.74 (s, 3H), 3.18-3.23 (m, 1H), 2.96-3.02 (m, 1H), 2.72-2.79 (m, 1H), 2.47-2.54 (m, 1H), 2.08-2.11 (m, 1H), 1.89-1.91 (m, 1H) Mass spectroscopy indicated MS (ESI): m/e 287 (M+H)⁺.

### Example 40

### Step C

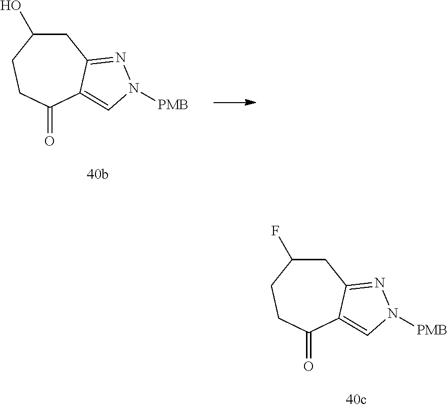

To a solution of compound 40b (150 mg, 0.52 mmol) in dry DCM (5 mL) was added DAST (191 mg, 1 mmol) at −78° C. under N₂. The mixture was stirred at −78° C. for 30 min then poured into H₂O (20 mL). Subsequently, aqueous NaHCO₃ was added until pH=8-9, then the mixture was extracted with aliquots of DCM (20 mL×3). The organic extracts were combined, dried over Na₂SO₄, filtered and concentrated. The residue was purified by preparative TLC (PE:EA=5:1) to afford product 40c (90 mg, 60% yield).

This compound was characterized by mass spectroscopy (MS) in accordance with the procedures described herein. Mass spectroscopy indicated MS (ESI): m/e 289 (M+H)⁺.

### Example 40

### Step D

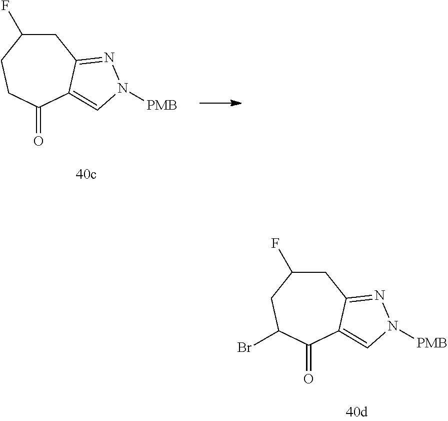

A mixture of compound 40c (90 mg, 0.31 mmol) and PhNMe₃Br₃ (116 mg, 0.31 mmol) in CHCl₃ (5 mL) was heated to reflux. Reflux was maintained for 30 min., then the mixture was cooled to r.t., diluted with DCM (40 mL), washed with brine (10 mL×2) then water (10 mL), dried over Na₂SO₄, filtered then concentrated to afford the product 40d (100 mg, 88%). The product thus obtained was used without further purification.

This compound was characterized by mass spectroscopy (MS) in accordance with the procedures described herein. Mass spectroscopy indicated MS (ESI): m/e 367, 369 (M+H)⁺.

### Example 40

### Step E

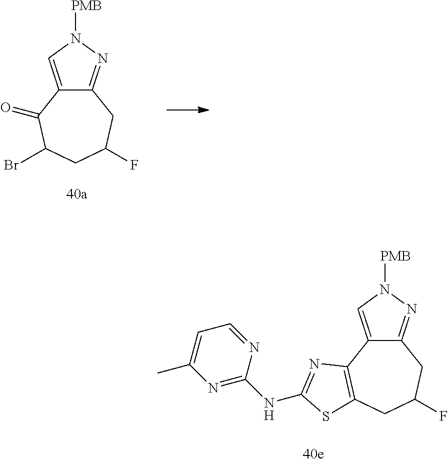

A mixture of compound 40d (100 mg, 0.27 mmol) and (4-methylpyrimidin-2-yl)thiourea (50 mg, 0.30 mmol) in n-BuOH (3 mL) was heated to reflux and maintained at reflux for 6 hours. Subsequently the mixture was cooled to r.t. and concentrated in vacuum to afford the product 40e (100 mg, 85%). The product thus obtained was used without further purification.

This compound was characterized by mass spectroscopy (MS) in accordance with the procedures described herein. Mass spectroscopy indicated MS (ESI): m/e 437 (M+H)⁺.

### Example 40

### Step F

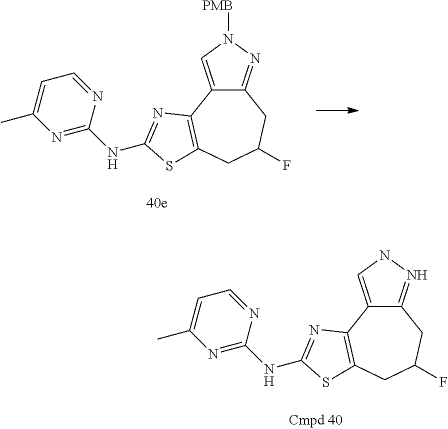

Compound 40e (100 mg, 0.23 mmol) in TFA (4 mL) was stirred at 100° C. under microwave conditions for 20 min then cooled to r.t., diluted with DCM and concentrated. The residue thus obtained was purified by preparative HPLC to give the desired product, Cmpd 40 (30 mg, 41%).

This compound was characterized by proton NMR (¹HNMR) and mass spectroscopy (MS) in accordance with the procedures described herein. Proton NMR yielded the following results: ¹H NMR (DMSO-d6, 400 MHz): δ 11.51 (br, 1H,), 8.43 (d, 1H, J=4.8 Hz), 7.74 (s, 1H), 6.89 (d, 1H, J=4.8 Hz), 5.16-5.29 (m, 1H), 3.23-3.34 (m, 2H), 2.41 (s, 3H). Mass spectroscopy indicated MS (ESI): m/e 317 (M+H)⁺.

We claim:

1) A compound of the formula: 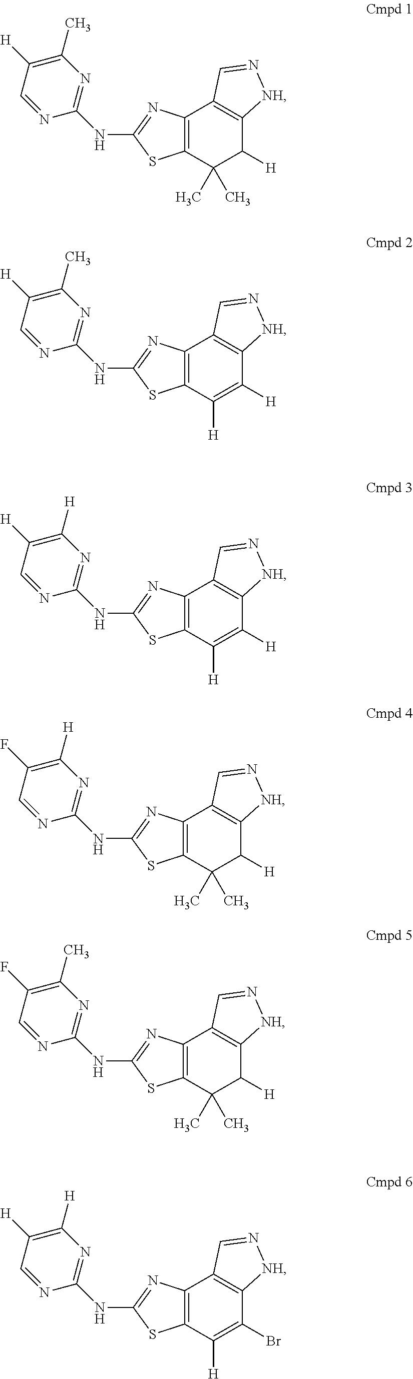 [SMILES: Cc1ccnc(Nc2nc3c(s2)C(C)(C)Cc2[nH]ncc2-3)n1 ; Cc1ccnc(Nc2nc3c(ccc4[nH]ncc43)s2)n1 ; c1cnc(Nc2nc3c(ccc4[nH]ncc43)s2)nc1 ; CC1(C)Cc2[nH]ncc2-c2nc(Nc3ncc(F)cn3)sc21 ; Cc1nc(Nc2nc3c(s2)C(C)(C)Cc2[nH]ncc2-3)ncc1F ; Brc1cc2sc(Nc3ncccn3)nc2c2cn[nH]c12] 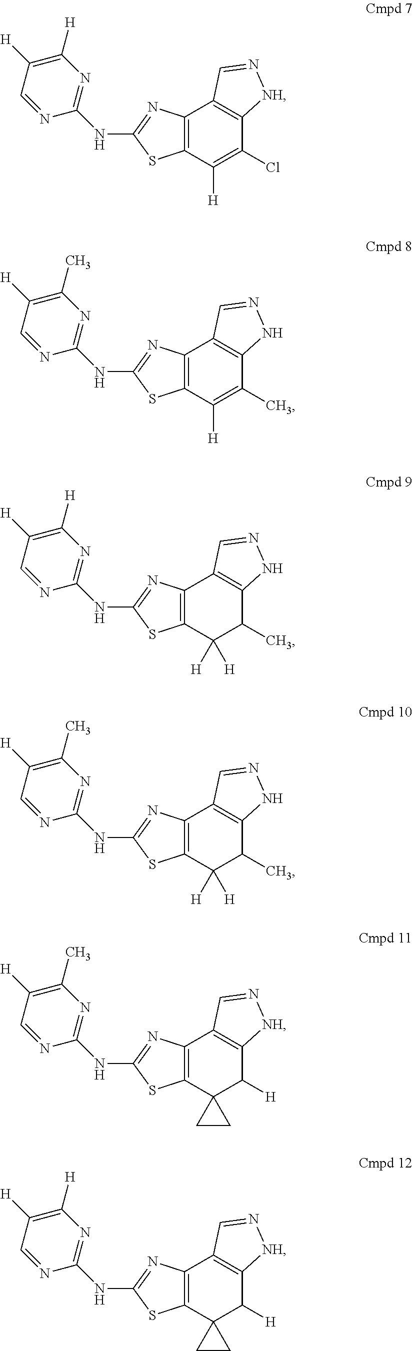 [SMILES: Clc1cc2sc(Nc3ncccn3)nc2c2cn[nH]c12 ; Cc1ccnc(Nc2nc3c(cc(C)c4[nH]ncc43)s2)n1 ; CC1Cc2sc(Nc3ncccn3)nc2-c2cn[nH]c21 ; Cc1ccnc(Nc2nc3c(s2)CC(C)c2[nH]ncc2-3)n1 ; Cc1ccnc(Nc2nc3c(s2)C2(CC2)Cc2[nH]ncc2-3)n1 ; c1cnc(Nc2nc3c(s2)C2(CC2)Cc2[nH]ncc2-3)nc1] 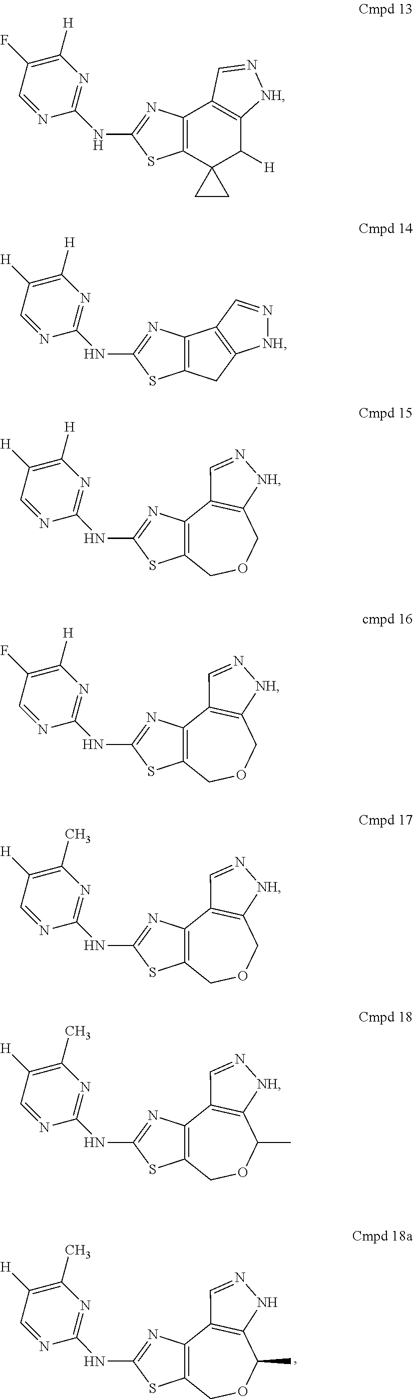 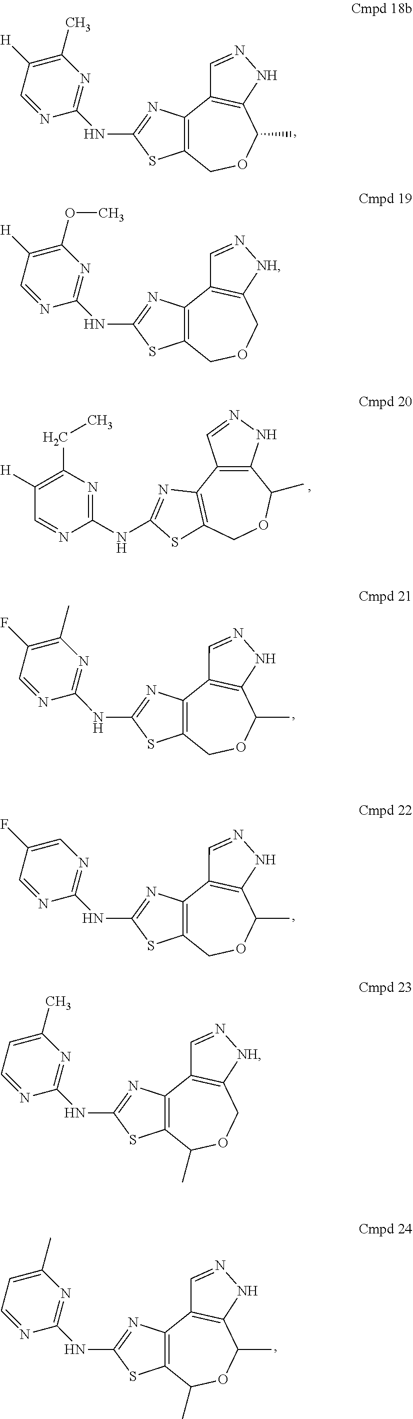 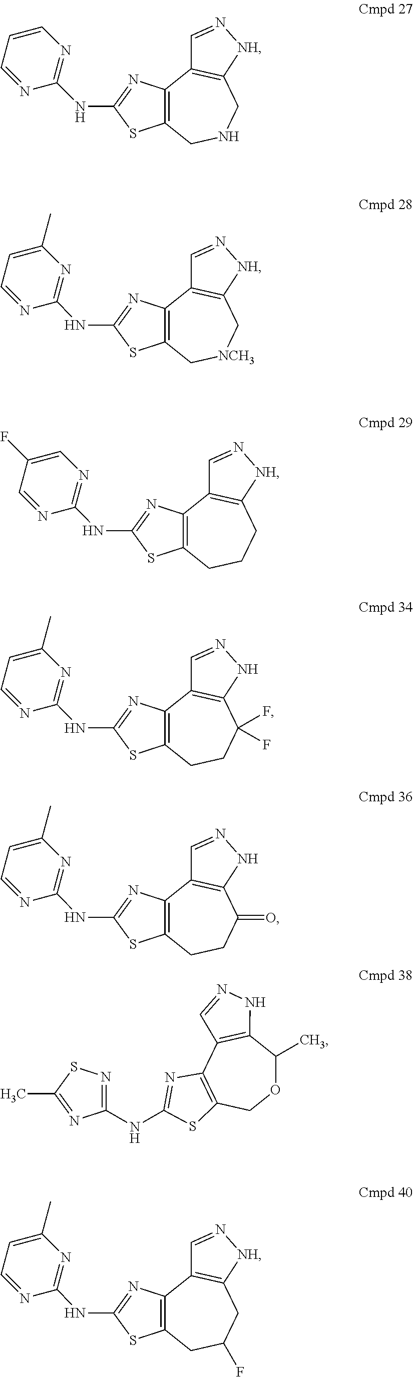
or a pharmaceutically acceptable salt thereof.

2) A pharmaceutical formulation comprising at least one compound of claim 1 or a pharmaceutically acceptable salt thereof.

3) Use of a compound of claim 1, or a pharmaceutically acceptable salt thereof, in the provision of a medicament for treating, preventing, or managing a disorder or disease which can be treated, prevented, or managed by administering an effective amount of an mGluR4 positive allosteric modulating compound.

4) A medicament of claim 3 formulated for use in the treatment or management of Parkinson's disease.

5) A method of treating, preventing, or managing a disorder or disease which can be treated, prevented, or managed by administering an effective amount of an mGluR4 positive allosteric modulating compound comprising administering to a mammalian patient in need of such a medicament of claim 3 in an effective amount in combination with an agent selected from the group consisting of: levodopa, levodopa with a selective extracerebral decarboxylase inhibitor, carbidopa, entacapone, a COMT inhibitor, a dopamine agonist, an anticholinergic, a cholinergic agonist, a butyrophenone neuroleptic agent, a diphenylbutylpiperidine neuroleptic agent, a heterocyclic dibenzazepine neuroleptic agent, an indolone neuroleptic agent, a phenothiazine neuroleptic agent, a thioxanthene neuroleptic agent, an NMDA receptor antagonist, an MAO-B inhibitor, an mGluR5 antagonist or an A_{2A} antagonist.
